SEQUENCE LISTING (35 sequences)

SEQ ID NO: 1 - protein, 529 aa, Listeria monocytogenes
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIE

SEQ ID NO: 2 - protein, 441 aa, Listeria monocytogenes
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYD

SEQ ID NO: 3 - protein, 416 aa, Listeria monocytogenes
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTD

SEQ ID NO: 4 - protein, 390 aa, Listeria monocytogenes
MRAMMVVFITANCITINPDIIFAATDSEDSSLNTDEWEEEKTEEQPSEVNTGPRYETAREVSSRDIKELEKSNKVRNTNKADLIAMLKEKAEKGPNINNNNSEQTENAAINEEASGADRPAIQVERRHPGLPSDSAAEIKKRRKAIASSDSELESLTYPDKPTKVNKKKVAKESVADASESDLDSSMQSADESSPQPLKANQQPFFPKVFKKIKDAGKWVRDKIDENPEVKKAIVDKSAGLIDQLLTKKKSEEVNASDFPPPPTDEELRLALPETPMLLGFNAPATSEPSSFEFPPPPTDEELRLALPETPMLLGFNAPATSEPSSFEFPPPPTEDELEIIRETASSLDSSFTRGDLASLRNAINRHSQNFSDFPPIPTEEELNGRGGRP

SEQ ID NO: 5 - DNA, 1170 bp, Listeria monocytogenes
atgcgtgcgatgatggtggttttcattactgccaattgcattacgattaaccccgacataatatttgcagcgacagatagcgaagattctagtctaaacacagatgaatgggaagaagaaaaaacagaagagcaaccaagcgaggtaaatacgggaccaagatacgaaactgcacgtgaagtaagttcacgtgatattaaagaactagaaaaatcgaataaagtgagaaatacgaacaaagcagacctaatagcaatgttgaaagaaaaagcagaaaaaggtccaaatatcaataataacaacagtgaacaaactgagaatgcggctataaatgaagaggcttcaggagccgaccgaccagctatacaagtggagcgtcgtcatccaggattgccatcggatagcgcagcggaaattaaaaaaagaaggaaagccatagcatcatcggatagtgagcttgaaagccttacttatccggataaaccaacaaaagtaaataagaaaaaagtggcgaaagagtcagttgcggatgcttctgaaagtgacttagattctagcatgcagtcagcagatgagtcttcaccacaacctttaaaagcaaaccaacaaccatttttccctaaagtatttaaaaaaataaaagatgcggggaaatgggtacgtgataaaatcgacgaaaatcctgaagtaaagaaagcgattgttgataaaagtgcagggttaattgaccaattattaaccaaaaagaaaagtgaagaggtaaatgcttcggacttcccgccaccacctacggatgaagagttaagacttgctttgccagagacaccaatgcttcttggttttaatgctcctgctacatcagaaccgagctcattcgaatttccaccaccacctacggatgaagagttaagacttgctttgccagagacgccaatgcttcttggttttaatgctcctgctacatcggaaccgagctcgttcgaatttccaccgcctccaacagaagatgaactagaaatcatccgggaaacagcatcctcgctagattctagttttacaagaggggatttagctagtttgagaaatgctattaatcgccatagtcaaaatttctctgatttcccaccaatcccaacagaagaagagttgaacgggagaggcggtagacca

SEQ ID NO: 6 - protein, 32 aa, Listeria monocytogenes
KENSISSMAPPASPPASPKTPIEKKHADEIDK

SEQ ID NO: 7 - protein, 14 aa, Listeria monocytogenes
KTEEQPSEVNTGPR

SEQ ID NO: 8 - protein, 28 aa, Listeria monocytogenes
KASVTDTSEGDLDSSMQSADESTPQPLK

SEQ ID NO: 9 - protein, 20 aa, Listeria monocytogenes
KNEEVNASDFPPPPTDEELR

SEQ ID NO: 10 - protein, 33 aa, Listeria monocytogenes
RGGIPTSEEFSSLNSGDFTDDENSETTEEEIDR

SEQ ID NO: 11 - protein, 17 aa, Streptococcus pyogenes
KQNTASTETTTTNEQPK

SEQ ID NO: 12 - protein, 17 aa, Streptococcus pyogenes
KQNTANTETTTTNEQPK

SEQ ID NO: 13 - protein, 98 aa, Human papillomavirus type 16
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 14 - protein, 105 aa, Human papillomavirus type 16
MHGPKATLQDIVLHLEPQNEIPVDLLCHEQLSDSEEENDEIDGVNHQHLPARRAEPQRHTMLCMCCKCEARIELVVESSADDLRAFQQLFLNTLSFVCPWCASQQ

SEQ ID NO: 15 - protein, 158 aa, Human papillomavirus type 16
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 16 - protein, 158 aa, Human papillomavirus type 16
MARFEDPTRRPYKLPDLCTELNTSLQDIEITCVYCKTVLELTEVFEFAFKDLFVVYRDSIPHAACHKCIDFYSRIRELRHYSDSVYGDTLEKLTNTGLYNLLIRCLRCQKPLNPAEKLRHLNEKRRFHNIAGHYRGQCHSCCNRARQERLQRRRETQV

SEQ ID NO: 17 - DNA, 22 bp, Artificial Sequence
  Primer
ggctcgagcatggagatacacc

SEQ ID NO: 18 - DNA, 28 bp, Artificial Sequence
  Primer
ggggactagtttatggtttctgagaaca

SEQ ID NO: 19 - DNA, 31 bp, Artificial Sequence
  Primer
gggggctagccctcctttgattagtatattc

SEQ ID NO: 20 - DNA, 28 bp, Artificial Sequence
  Primer
ctccctcgagatcataatttacttcatc

SEQ ID NO: 21 - DNA, 55 bp, Artificial Sequence
  Primer
gactacaaggacgatgaccgacaagtgataacccgggatctaaataaatccgttt

SEQ ID NO: 22 - DNA, 27 bp, Artificial Sequence
  Primer
cccgtcgaccagctcttcttggtgaag

SEQ ID NO: 23 - DNA, 25 bp, Artificial Sequence
  Primer
gcggatcccatggagatacacctac

SEQ ID NO: 24 - DNA, 22 bp, Artificial Sequence
  Primer
gctctagattatggtttctgag

SEQ ID NO: 25 - DNA, 31 bp, Artificial Sequence
  Primer
ggggtctagacctcctttgattagtatattc

SEQ ID NO: 26 - DNA, 45 bp, Artificial Sequence
  Primer
atcttcgctatctgtcgccgcggcgcgtgcttcagtttgttgcgc

SEQ ID NO: 27 - DNA, 45 bp, Artificial Sequence
  Primer
gcgcaacaaactgaagcagcggccgcggcgacagatagcgaagat

SEQ ID NO: 28 - DNA, 42 bp, Artificial Sequence
  Primer
tgtaggtgtatctccatgctcgagagctaggcgatcaatttc

SEQ ID NO: 29 - DNA, 42 bp, Artificial Sequence
  Primer
ggaattgatcgcctagctctcgagcatggagatacacctaca

SEQ ID NO: 30 - DNA, 42 bp, Artificial Sequence
  Primer
aaacggatttatttagatcccgggttatggtttctgagaaca

SEQ ID NO: 31 - DNA, 42 bp, Artificial Sequence
  Primer
tgttctcagaaaccataacccgggatctaaataaatccgttt

SEQ ID NO: 32 - DNA, 28 bp, Artificial Sequence
  Primer
gggggtcgaccagctcttcttggtgaag

SEQ ID NO: 33 - protein, 9 aa, Human papillomavirus type 16
RAHYNIVTF

SEQ ID NO: 34 - protein, 9 aa, Human immunodeficiency virus type 1
AMQMLKETI

SEQ ID NO: 35 - protein, 9 aa, Listeria monocytogenes
GYKDGNEYI

(19) United States
(12) Patent Application Publication
(10) Pub. No.: US 2018/0265879 A1
(43) Pub. Date: Sep. 20, 2018

(54) MANUFACTURING METHOD OF AN IMMUNOTHERAPEUTIC FORMULATION COMPRISING A RECOMBINANT LISTERIA STRAIN

(71) Applicants: Advaxis, Inc., Princeton, NJ (US); The Trustees of the University of Pennsylvania, Philadelphia, PA (US)

(72) Inventors: Anu Wallecha, Yardley, PA (US); Yvonne Paterson, Philadelphia, PA (US)

(21) Appl. No.: 15/760,263

(22) PCT Filed: Sep. 13, 2016
(86) PCT No.: PCT/US2016/051525

§ 371 Date: Mar. 15, 2018

Related U.S. Application Data
(60) Provisional application No. 62/218,884, filed on Sep. 15, 2015.

(51) Int. Cl. C12N 15/74; C12P 21/00; C07K 14/195; C07K 14/025; C07K 19/00; A61K 39/12

(52) U.S. Cl. CPC C12N 15/74; C12P 21/00; C07K 14/195; A61P 35/00; C07K 19/00; A61K 39/12; C07K 14/025

(57) ABSTRACT

The present invention discloses a process for manufacturing a formulation comprising a drug substance, said drug substance comprising a recombinant Listeria comprising a human papilloma virus (HPV) antigen fused to a Listeriolysin O (LLO) protein fragment. The invention further discloses methods of using treating, protecting against, and inducing an immune response against cervical cancer comprising administration of the recombinant Listeria strain. The present invention also provides methods for inducing an anti-E7 CTL response in a human subject and treating HPV-mediated diseases, disorders, and symptoms, comprising administration of the recombinant Listeria strain.

### FIELD OF INVENTION

The present invention discloses a process for manufacturing a formulation comprising a drug substance, said drug substance comprising a recombinant Listeria comprising a human papilloma virus (HPV) antigen fused to a Listeriolysin O (LLO) protein fragment. The invention further discloses methods of using treating, protecting against, and inducing an immune response against cervical cancer comprising administration of the recombinant Listeria strain. The present invention also provides methods for inducing an anti-E7 CTL response in a human subject and treating HPV-mediated diseases, disorders, and symptoms, comprising administration of the recombinant Listeria strain.

### BACKGROUND OF THE INVENTION

Worldwide, approximately 500,000 cases of cervical cancer are diagnosed each year. Cancer of the cervix (cervical cancer) begins in the lining of the cervix. Normal cervical cells gradually develop pre-cancerous changes that turn into cancer. Several terms are used to describe these pre-cancerous changes, including cervical intraepithelial neoplasia (CIN), squamous intraepithelial lesion (SIL), and neoplasia in situ, dysplasia.

There are 2 major types of cervical cancers: squamous cell carcinoma and adenocarcinoma. Cervical cancers and cervical precancers are classified by microscopic appearance. About 80%-90% of cervical cancers are squamous cell carcinomas, which are composed of cells that resemble the flat, thin cells called squamous cells that cover the surface of the endocervix. Squamous cell carcinomas most often begin where the ectocervix joins the endocervix.

The remaining 10%-20% of cervical cancers are adenocarcinomas. Adenocarcinomas are becoming more common in women born in the last 20 to 30 years. Cervical adenocarcinoma develops from the mucus-producing gland cells of the endocervix. Less commonly, cervical cancers have features of both squamous cell carcinomas and adenocarcinomas. These are called “adenosquamous carcinomas” or “mixed carcinomas.”

Improved therapies for cervical cancers are urgently needed in the art. The invention disclosed herein provides a novel process for manufacturing a formulation comprising a drug substance (recombinant Listeria strain) for inducing an immune response against, treating, and protecting from cervical cancer.

### SUMMARY OF THE INVENTION

In one aspect, the invention relates to process for the manufacturing of a formulation comprising a drug substance, said drug substance comprising a recombinant Listeria, said recombinant Listeria strain comprising a nucleic acid molecule, wherein said nucleic acid molecule comprises an open reading frame encoding a recombinant polypeptide, said recombinant polypeptide comprising a human papilloma virus (HPV) antigen fused to a Listeriolysin O (LLO) polypeptide, wherein the process comprises the steps of:
a) Preparing fermentation media and adding said fermentation media into a fermenter system.
   i. wherein said preparing further comprises adding an antibiotic agent.
b) Adding a working cell bank (WCB) comprising said drug substance into said fermenter system.
   i. initiating the fermentation process until a target optical density (OD) of said drug substance is reached.
c) Aseptically connecting said fermenter system to a filtration system and concentrating said drug substance to a desired weight by collecting a calculated amount of permeate.
d) Obtaining a retentate solution comprising said drug substance from step c) and exchanging the fermentation media with an appropriate formulation for human use.
   i. measuring the OD of said formulation buffer solution comprising said drug substance.
e) Diluting said drug substance using said formulation buffer to a target OD, thereby forming a bulk drug substance (BDS).
f) Transferring said BDS into product bags.
   i. aseptically filling said from said bags BDS into vials for storage or for drug preparation and administration to a subject.

In another related aspect, disclosed herein are methods of treating, protecting against, and inducing an immune response against cervical cancer, comprising the step of administering to a subject a recombinant Listeria strain, comprising a fusion peptide that comprises an LLO fragment and an E7 and/or E6 antigen. The present invention also provides methods for inducing an anti-E7 CTL response in a human subject and treating HPV-mediated diseases, disorders, and symptoms, comprising administration of the recombinant Listeria strain.

Other features and advantages of the present invention will become apparent from the following detailed description examples and figures. It should be understood, however, that the detailed description and the specific examples while indicating embodiments of the invention are given by way of illustration only, since various changes and modifications within the spirit and scope of the invention will become apparent to those skilled in the art from this detailed description.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1A-1B. Lm-E7 and Lm-LLO-E7 use different expression systems to express and secrete E7. Lm-E7 was generated by introducing a gene cassette into the orfZ domain of the L. monocytogenes genome (FIG. 1A). The hly promoter drives expression of the hly signal sequence and the first five amino acids (AA) of LLO followed by HPV-16 E7. FIG. 1B. Lm-LLO-E7 was generated by transforming the prfA-strain XFL-7 with the plasmid pGG-55. pGG-55 has the hly promoter driving expression of a nonhemolytic fusion of LLO-E7. pGG-55 also contains the prfA gene to select for retention of the plasmid by XFL-7 in vivo.

FIG. 2. Lm-E7 and Lm-LLO-E7 secrete E7. Lm-Gag (lane 1), Lm-E7 (lane 2), Lm-LLO-NP (lane 3), Lm-LLO-E7 (lane 4), XFL-7 (lane 5), and 10403S (lane 6) were grown overnight at 37° C. in Luria-Bertoni broth. Equivalent numbers of bacteria, as determined by OD at 600 nm absorbance, were pelleted and 18 ml of each supernatant was TCA precipitated. E7 expression was analyzed by Western blot. The blot was probed with an anti-E7 mAb, followed by HRP-conjugated anti-mouse (Amersham), then developed using ECL detection reagents.

FIG. 3. Tumor immunotherapeutic efficacy of LLO-E7 fusions. Tumor size in millimeters in mice is shown at 7, 14, 21, 28 and 56 days post tumor-inoculation. Naive mice: open-circles; Lm-LLO-E7: filled circles; Lm-E7: squares; Lm-Gag: open diamonds; and Lm-LLO-NP: filled triangles.

FIG. 4. Splenocytes from Lm-LLO-E7-immunized mice proliferate when exposed to TC-1 cells. C57BL/6 mice were immunized and boosted with Lm-LLO-E7, Lm-E7, or control rLm strains. Splenocytes were harvested 6 days after the boost and plated with irradiated TC-1 cells at the ratios shown. The cells were pulsed with ³H thymidine and harvested. Cpm is defined as (experimental cpm)—(no-TC-1 control).

FIG. 5A-5B. FIG. 5A. Western blot demonstrating that Lm-ActA-E7 secretes E7. Lane 1: Lm-LLO-E7; lane 2: Lm-ActA-E7.001; lane 3; Lm-ActA-E7-2.5.3; lane 4: Lm-ActA-E7-2.5.4. FIG. 5B. Tumor size in mice administered Lm-ActA-E7 (rectangles), Lm-E7 (ovals), Lm-LLO-E7 (X), and naive mice (non-vaccinated; solid triangles).

FIG. 6A-6C. FIG. 6A. schematic representation of the plasmid inserts used to create 4 LM vaccines. Lm-LLO-E7 insert contains all of the Listeria genes used. It contains the hly promoter, the first 1.3 kb of the hly gene (which encodes the protein LLO), and the HPV-16 E7 gene. The first 1.3 kb of hly includes the signal sequence (ss) and the PEST region. Lm-PEST-E7 includes the hly promoter, the signal sequence, and PEST and E7 sequences but excludes the remainder of the truncated LLO gene. Lm-ΔPEST-E7 excludes the PEST region, but contains the hly promoter, the signal sequence, E7, and the remainder of the truncated LLO.

Lm-E7epi has only the hly promoter, the signal sequence, and E7. FIG. 6B. Top panel: Listeria constructs containing PEST regions induce tumor regression. Bottom panel: Average tumor sizes at day 28 post-tumor challenge in 2 separate experiments. FIG. 6C. Listeria constructs containing PEST regions induce a higher percentage of E7-specific lymphocytes in the spleen. Average and SE of data from 3 experiments are depicted.

FIG. 7A-7B. FIG. 7A. Induction of E7-specific IFN-gamma-secreting CD8⁺ T cells in the spleens and the numbers penetrating the tumors, in mice administered TC-1 tumor cells and subsequently administered Lm-E7, Lm-LLO-E7, Lm-ActA-E7, or no vaccine (naive). FIG. 7B. Induction and penetration of E7 specific CD8⁺ cells in the spleens and tumors of the mice described for FIG. 7A.

FIG. 8A-8B. Listeria constructs containing PEST regions induce a higher percentage of E7-specific lymphocytes within the tumor. FIG. 8A. representative data from 1 experiment. FIG. 8B. average and SE of data from all 3 experiments.

FIG. 9. E6/E7 transgenic mice develop tumors in their thyroid, where the E7 gene is expressed. Mice were sacrificed at 3 months and had their thyroids removed, sectioned, and stained by hematoxylin and eosin. A. Left panel: normal thyroid at 20× magnification. Follicles are of normal size and lined with cuboidal cells with abundant pink cytoplasm (arrow). Right panel: E6/E7 transgenic mouse thyroid. Note the greatly enlarged follicles because of the increased production of colloid. The cuboidal cells lining the follicles are smaller with very little cytoplasm.

FIG. 10A-10B. E7 message is expressed in the thyroid and medullary thymic epithelial cells of the E6/E7 transgenic mouse. FIG. 10A. Tissue-specific expression of the E7 transgene is detected in the thyroid only but not the liver, spleen, or whole thymus. Lane 1: Liver; Lane 2: Spleen; Lane 3: Thyroid; Lane 4: Whole Thymus. FIG. 10B. Medullary thymic epithelial cells (mTECs) express E7. RT-PCR results are as shown for equivalent amounts of cDNA loaded for 40 cycles. Lane 5: Cathepsin S; Lane 6: E7; Lane 7: Actin; Lane 8: Negative Control.

FIG. 11. RAHYNIVTF peptide plus CpG adjuvant does not protect against TC-1 challenge in E6/E7 transgenic mice. Two groups of transgenic mice received either E7 peptide plus adjuvant or PBS. A third group of wild type C57B1/6 control mice received E7 peptide plus adjuvant. The mice were vaccinated twice intraperitoneally (i.p.), 7 days apart and challenged with 5×10⁴ TC-1 cells 7 days later. Tumors were measured every 5 days until unimmunized mice needed to be sacrificed. Error bars: standard deviations from the mean value.

FIG. 12A-12B. Vaccines of the present invention induce regression of solid tumors in the E6/E7 transgenic mice in wild-type mice and transgenic mice immunized with LM-LLO-E7 (FIG. 12A), or LM-ActA-E7 (FIG. 12B), left naïve, or treated with LM-NP (control).

FIG. 13A-13B. FIG. 13A. IV immunization of LM-LLO-E7 is effective at inducing the regression of established tumors at doses as low as 1×10⁶ CFU per mouse. FIG. 13B. Tumors loads for the 2 cohorts in the LM-LLO-E7 clinical trial.

FIG. 14A-14B. FIG. 14A. Effect of passaging on bacterial load (virulence) of recombinant Listeria vaccine vectors. Top panel. Lm-Gag. Bottom panel. Lm-LLO-E7. FIG. 14B. Effect of passaging on bacterial load of recombinant Lm-E7 in the spleen. Average CFU of live bacteria per milliliter of spleen homogenate from four mice is depicted.

FIG. 15. Induction of antigen-specific CD8⁺ T-cells for HIV-Gag and LLO after administration of passaged Lm-Gag versus unpassaged Lm-Gag. Mice were immunized with 10³ (A, B, E, F) or 10⁵ (C, D, G, H) CFU passaged Listeria vaccine vectors, and antigen-specific T-cells were analyzed. B, D, F, H: unpassaged Listeria vaccine vectors. A-D immune response to MHC class I HIV-Gag peptide. E-H: immune response to an LLO peptide. I: splenocytes from mice immunized with 10⁵ CFU passaged Lm-Gag stimulated with a control peptide from HPV E7.

FIG. 16A-16C. FIG. 16A. Plasmid isolation throughout LB stability study. FIG. 16B. Plasmid isolation throughout TB stability study. FIG. 16C. Quantitation of TB stability study.

FIG. 17. Numbers of viable bacteria chloramphenicol (CAP)-resistant and CAP-sensitive colony-forming units (CFU) from bacteria grown in LB. Dark bars: CAP⁺; white bars: CAP⁻. The two dark bars and two white bars for each time point represent duplicate samples.

FIG. 18. Numbers of viable bacteria CAP-resistant and CAP-sensitive CFU from bacteria grown in TB. Dark bars: CAV⁺; white bars: CAP. The two dark bars and two white bars for each time point represent duplicate samples.

FIG. 19. Manufacturing process of drug substance (ADXS11-001). ADXS11-001 flow diagram.

### DETAILED DESCRIPTION OF THE INVENTION

In one embodiment, disclosed herein is a process for manufacturing a formulation comprising a drug substance, said drug substance comprising a recombinant Listeria, said recombinant Listeria strain comprising a nucleic acid molecule, wherein said nucleic acid molecule comprises an open reading frame encoding a recombinant polypeptide, said recombinant polypeptide comprising a human papilloma virus (HPV) antigen fused to a Listeriolysin O (LLO) polypeptide, wherein the process comprises the steps of:
a) Preparing fermentation media and adding said fermentation media into a fermenter system.
   i. wherein said preparing further comprises adding an antibiotic agent in solution.
b) Adding a working cell bank (WCB) comprising said drug substance into said fermenter system.
   i. initiating the fermentation process until a target optical density (OD) of said drug substance is reached.
c) Aseptically connecting said fermenter system to a filtration system and concentrating said drug substance to a desired weight by collecting a calculated amount of permeate.
   i. wherein said concentrating step in c) is to 2-10 fold concentration.
d) Obtaining a retentate solution comprising said drug substance from step c) exchanging the fermentation media with an appropriate formulation for human use.
   i. measuring the OD of said formulation buffer solution comprising said drug substance.
e) Diluting said drug substance using said formulation buffer to a target OD, thereby forming a bulk drug substance (BDS).
g) Transferring said BDS into product bags.
   i. aseptically filling said from said bags BDS into vials for storage or for drug preparation and administration to a subject.

In one embodiment, a drug substance disclosed herein comprises a recombinant Listeria also disclosed herein. In another embodiment, a drug substance disclosed is a recombinant Listeria also disclosed herein. In one embodiment, the recombinant Listeria strain of disclosed herein is a recombinant Listeria monocytogenes strain. In another embodiment, the Listeria strain is a recombinant Listeria seeligeri strain. In another embodiment, the Listeria strain is a recombinant Listeria grayi strain. In another embodiment, the Listeria strain is a recombinant Listeria ivanovii strain. In another embodiment, the Listeria strain is a recombinant Listeria murrayi strain. In another embodiment, the Listeria strain is a recombinant Listeria welshimeri strain. In another embodiment, the Listeria strain is a recombinant strain of any other Listeria species known in the art.

In another embodiment, the recombinant Listeria disclosed herein comprises a nucleic acid molecule in a plasmid in said recombinant Listeria. In another embodiment, said plasmid is stably maintained in said recombinant Listeria. In another embodiment, said plasmid lacks antibiotic resistance genes. In another embodiment, said plasmid is an integrative plasmid. In another embodiment, said plasmid is an extrachrosomal or episomal plasmid.

In one embodiment, a recombinant Listeria disclosed herein comprises a mutation, deletion or inactivation of an endogenous prfA gene.

In one embodiment, a plasmid disclosed herein comprises an open reading frame encoding a mutant prfA gene that complements said mutation or deletion in said endogenous prfA gene in said recombinant Listeria or restores partial prfA function in said recombinant Listeria. In one embodiment, said mutant prfA protein that complements said mutation or deletion in said endogenous prfA gene in said recombinant Listeria or restores partial prfA function in said recombinant Listeria comprises a D133V amino acid mutation.

In one embodiment, a recombinant polypeptide disclosed herein is expressed by a recombinant Listeria.

In another embodiment, a recombinant Listeria strain of the present invention has been passaged through an animal host. In another embodiment, the passaging maximizes efficacy of the strain as a vaccine vector. In another embodiment, the passaging stabilizes the immunogenicity of the Listeria strain. In another embodiment, the passaging stabilizes the virulence of the Listeria strain. In another embodiment, the passaging increases the immunogenicity of the Listeria strain. In another embodiment, the passaging increases the virulence of the Listeria strain. In another embodiment, the passaging removes unstable sub-strains of the Listeria strain. In another embodiment, the passaging reduces the prevalence of unstable sub-strains of the Listeria strain. In another embodiment, the Listeria strain contains a genomic insertion of the gene encoding the antigen-containing recombinant peptide. In another embodiment, the Listeria strain carries a plasmid comprising the gene encoding the antigen-containing recombinant peptide. In another embodiment, the passaging is performed as described herein (e.g. in Example 12). In another embodiment, the passaging is performed by any other method known in the art.

In another embodiment, the recombinant Listeria strain utilized in methods of the present invention has been stored in a frozen cell bank prior to adding into a fermenter disclosed herein. In another embodiment, the recombinant Listeria strain has been stored in a lyophilized cell bank prior to adding into a fermenter disclosed herein.

In another embodiment, the cell bank of methods and compositions of the present invention is a master cell bank (MCB). In another embodiment, the cell bank is a working cell bank (WCB). In another embodiment, the cell bank is Good Manufacturing Practice (GMP) cell bank. In another embodiment, the cell bank is intended for production of clinical-grade material. In another embodiment, the cell bank conforms to regulatory practices for human use. In another embodiment, the cell bank is any other type of cell bank known in the art.

In one embodiment, 60 mL bags containing the 5-20 mL of the ADXS11-001 Working Cell Bank (WCB) are thawed prior to adding a drug substance disclosed herein into the fermenter system disclosed herein. In another embodiment, 40-80 mL bags containing the 5-20 mL of the ADXS11-001 Working Cell Bank (WCB) are thawed prior to adding a drug substance disclosed herein into the fermenter system disclosed herein. In another embodiment, 5 ml of the ADXS11-001 Working Cell Bank (WCB) are present in the bags. In another embodiment, 10 ml of the ADXS11-001 Working Cell Bank (WCB) are present in the bags. In another embodiment, 15 ml of the ADXS11-001 Working Cell Bank (WCB) are present in the bags. In another embodiment, 20 ml of the ADXS11-001 Working Cell Bank (WCB) are present in the bags.

“Good Manufacturing Practices” are defined, in another embodiment, by (21 CFR 210-211) of the United States Code of Federal Regulations. In another embodiment, “Good Manufacturing Practices” are defined by other standards for production of clinical-grade material or for human consumption; e.g. standards of a country other than the United States.

In another embodiment, a recombinant Listeria strain utilized in methods of the present invention is from a batch of immunotherapy doses.

In another embodiment, a recombinant Listeria strain utilized in methods of the present invention is from a frozen stock produced by the process disclosed herein.

In another embodiment, a recombinant Listeria strain utilized in methods of the present invention is from a lyophilized stock produced by the process disclosed herein.

In another embodiment, a cell bank, frozen stock, or batch of vaccine doses of the present invention exhibits viability upon thawing of greater than 90%. In another embodiment, the thawing follows storage for cryopreservation or frozen storage for 2 hours. In another embodiment, the thawing follows storage for cryopreservation or frozen storage for 6 hours. In another embodiment, the thawing follows storage for cryopreservation or frozen storage for 12 hours. In another embodiment, the thawing follows storage for cryopreservation or frozen storage for 24 hours. In another embodiment, the storage is for 2 days. In another embodiment, the storage is for 3 days. In another embodiment, the storage is for 4 days. In another embodiment, the storage is for 1 week. In another embodiment, the storage is for 2 weeks. In another embodiment, the storage is for 3 weeks. In another embodiment, the storage is for 1 month. In another embodiment, the storage is for 2 months. In another embodiment, the storage is for 3 months. In another embodiment, the storage is for 5 months. In another embodiment, the storage is for 6 months. In another embodiment, the storage is for 9 months. In another embodiment, the storage is for 1 year.

In another embodiment, a cell bank, frozen stock, or batch of vaccine doses of the present invention is cryopreserved by a method that comprises growing a culture of the Listeria strain in a nutrient media, freezing the culture in a solution comprising an antifreeze agent, and storing the Listeria strain at below −20 degrees Celsius. In another embodiment, the antifreeze agent is propylene glycol. In another embodiment, the antifreeze agent is ethylene glycol. In another embodiment, the antifreeze agent is glycerol. In another embodiment, the antifreeze agent is sucrose. In another embodiment, the temperature is about −70 degrees Celsius. In another embodiment, the temperature is about ⁻70-⁻80 degrees Celsius.

In another embodiment, a cell bank, frozen stock, or batch of vaccine doses of the present invention is cryopreserved by a method that comprises growing a culture of the Listeria strain in a defined media, freezing the culture in a solution comprising an antifreeze agent, and storing the Listeria strain at below −20 degrees Celsius. In another embodiment, the antifreeze agent is propylene glycol. In another embodiment, the antifreeze agent is ethylene glycol. In another embodiment, the antifreeze agent is glycerol. In another embodiment, the antifreeze agent is sucrose. In another embodiment, the temperature is about −70 degrees Celsius. In another embodiment, the temperature is about ⁻70-⁻80 degrees Celsius. In another embodiment, any defined microbiological media of the present invention may be used in this method.

In another embodiment of methods and compositions disclosed herein, the culture (e.g. the culture of a Listeria vaccine strain that is used to produce a batch of Listeria vaccine doses) is inoculated from a cell bank. In another embodiment, the culture is inoculated from a frozen stock. In another embodiment, the culture is inoculated from a starter culture. In another embodiment, the culture is inoculated from a colony. In another embodiment, the culture is inoculated at mid-log growth phase. In another embodiment, the culture is inoculated at approximately mid-log growth phase. In another embodiment, the culture is inoculated at another growth phase. In another embodiment, the WCB is removed from ≤−70° C. storage and thawed at room temperature prior to adding into a fermenter system.

In another embodiment of methods and compositions disclosed herein, the solution used for freezing contains ethylene glycol, propylene glycol, glycerol or sucrose in an amount of 1-20%. In another embodiment, the amount is 2%. In another embodiment, the amount is 20%. In another embodiment, the amount is 1%. In another embodiment, the amount is 1.5%. In another embodiment, the amount is 3%. In another embodiment, the amount is 4%. In another embodiment, the amount is 5%. In another embodiment, the amount is 2%. In another embodiment, the amount is 2%. In another embodiment, the amount is 7%. In another embodiment, the amount is 9%. In another embodiment, the amount is 10%. In another embodiment, the amount is 12%. In another embodiment, the amount is 14%. In another embodiment, the amount is 16%. In another embodiment, the amount is 18%. In another embodiment, the amount is 222%. In another embodiment, the amount is 25%. In another embodiment, the amount is 30%. In another embodiment, the amount is 35%. In another embodiment, the amount is 40%.

In another embodiment, the solution used for freezing contains another colligative additive or additive with anti-freeze properties, in place of glycerol. In another embodiment, the solution used for freezing contains another colligative additive or additive with anti-freeze properties, in addition to glycerol. In another embodiment, the additive is mannitol. In another embodiment, the additive is DMSO. In another embodiment, the additive is sucrose. In another embodiment, the additive is any other colligative additive or additive with anti-freeze properties that is known in the art.

In another embodiment, the nutrient media utilized for growing a culture of a Listeria strain is LB. In another embodiment, the nutrient media is TB. In another embodiment, the nutrient media is a defined media. In another embodiment, the nutrient media is peptone based. In another embodiment, the nutrient media is dextrose based. In another embodiment, the nutrient media is any other type of nutrient media known in the art. In another embodiment of the methods and compositions disclosed herein, the step of growing is performed with a shake flask. In another embodiment, the flask is a baffled shake flask. In another embodiment, the growing is performed with a batch fermenter. In another embodiment, the growing is performed with a stirred tank or flask. In another embodiment, the growing is performed with an airlift fermenter. In another embodiment, the growing is performed with a fed batch. In another embodiment, the growing is performed with a continuous cell reactor. In another embodiment, the growing is performed with a Wave Bioreactor. In another embodiment, the growing is performed with a Bioreactor that uses wave-like motion. In another embodiment, the growing is performed with an immobilized cell reactor. In another embodiment, the growing is performed with any other means of growing bacteria that is known in the art.

It will be appreciated by a skilled artisan that the terms “reactor,” “bioreactor,” “fermenter,” and “fermentation system” are used interchangeably herein. In one embodiment, the fermentation system disclosed herein is a disposable system. In another embodiment, the fermentation system is any fermentation system known in the art.

In one embodiment, the term “bioreactor,” “fermenter” and “fermenter system” are used interchangeably herein. In one embodiment, the fermenter disclosed herein is aseptically sampled to measure Optical Density, pH and Viable Cell Count off-line following transfer of the WCB into said fermenter.

In one embodiment, the fermenter is set at a specific rocking rate. In another embodiment, the bioreactor is set to rock 10-30 times per minute. In another embodiment, the bioreactor is set to rock 20-40 times per minute. In another embodiment, the bioreactor is set to rock 50-80 times per minute.

In another embodiment, the fermenter is set at a specific rocking angle. In another embodiment, the fermenter is set to rock at a 2-10° angle. In another embodiment, the fermenter is set to rock at a 11-20° angle. In another embodiment, the fermenter is set to rock at a 21-40° angle. In another embodiment, the fermenter is set to rock at a 41-60° angle. In another embodiment, the fermenter is set to rock at a 61-80° angle. In another embodiment, the fermenter is set to rock at an 80-90° angle.

In one embodiment, the fermentation process is controlled by monitoring the dissolved oxygen (dO2) levels, the pH and temperature within the fermentation system. In another embodiment, the dO2 is monitored during the exponential growth.

In one embodiment, the fermentation process is controlled by monitoring the dissolved oxygen (dO2) levels, the pH and temperature within the fermentation system at intervals of up to 20 minutes. In another embodiment, the dO2 is monitored during the exponential growth at intervals of up to 20 minutes. In one embodiment, the fermentation process is controlled by monitoring the dissolved oxygen (dO2) levels, the pH and temperature within the fermentation system at intervals of up to 40 minutes. In another embodiment, the dO2 is monitored during the exponential growth at intervals of up to 40 minutes. In one embodiment, the fermentation process is controlled by monitoring the dissolved oxygen (dO2) levels, the pH and temperature within the fermentation system at intervals of up to 60 minutes. In another embodiment, the dO2 is monitored during the exponential growth at intervals of up to 60 minutes.

In one embodiment, the fermentation process is controlled by monitoring the dissolved oxygen (dO2) levels, the pH and temperature within the fermentation system at intervals of more than 60 minutes. In another embodiment, the dO2 is monitored during the exponential growth at intervals of more than 60 minutes.

In one embodiment, the fermentation process is sampled to measure the optical density (OD 600 nm), the pH, and the viable cell count (VCC).

In one embodiment, the fermentation process is sampled to measure the optical density (OD 600 nm), the pH, and the viable cell count (VCC) at intervals of up to 20 minutes. In another embodiment, the fermentation process is sampled to measure the optical density (OD 600 nm), the pH, and the viable cell count (VCC) at intervals of up to 40 minutes. In another embodiment, the fermentation process is sampled to measure the optical density (OD 600 nm), the pH, and the viable cell count (VCC) at intervals of up to 60 minutes. In another embodiment, the fermentation process is sampled to measure the optical density (OD 600 nm), the pH, and the viable cell count (VCC) at intervals of more than 60 minutes.

In another embodiment, the pH of the fermentation process disclosed herein is controlled using an alkylating agent. In another embodiment, the alkylating agent is selected from one or more of the following sodium hydroxide, potassium carbonate, potassium hydroxid, sodium carbonate, sodium metasilicate, trisodium phosphate. In another embodiment, a constant pH is maintained during growth of the culture in a fermenter system. In another embodiment, the pH is maintained at about 7.0. In another embodiment, the pH is about 6. In another embodiment, the pH is about 6.5. In another embodiment, the pH is about 7.5. In another embodiment, the pH is about 8. In another embodiment, the pH is 6.5-7.5. In another embodiment, the pH is 6-8. In another embodiment, the pH is 6-7. In another embodiment, the pH is 7-8.

In another embodiment, a constant temperature is maintained during growth of the culture. In another embodiment, the temperature is maintained at about 37° C. In another embodiment, the temperature is 37° C. In another embodiment, the temperature is 25° C. In another embodiment, the temperature is 27° C. In another embodiment, the temperature is 28° C. In another embodiment, the temperature is 30° C. In another embodiment, the temperature is 32° C. In another embodiment, the temperature is 33° C. In another embodiment, the temperature is 34° C. In another embodiment, the temperature is 35° C. In another embodiment, the temperature is 36° C. In another embodiment, the temperature is 38° C. In another embodiment, the temperature is 39° C. In another embodiment, the temperature is 40° C. In another embodiment, the temperature is 41° C. In another embodiment, the temperature is 42° C. In another embodiment, the temperature is 43° C. In another embodiment, the temperature is 44° C. In another embodiment, the temperature is 45° C. In another embodiment, the temperature is about 30° C.-45° C.

In one embodiment, a (dO₂) level is monitored during the exponential growth phase of the recombinant Listeria culture. In another embodiment, the dO₂ concentration is maintained during growth of the culture. In another embodiment, a constant dissolved oxygen (dO₂) concentration is maintained during growth of the culture. In another embodiment, the dissolved oxygen concentration is maintained at 20% of saturation. In another embodiment, the concentration is 15% of saturation. In another embodiment, the concentration is 16% of saturation. In another embodiment, the concentration is 18% of saturation. In another embodiment, the concentration is 22% of saturation. In another embodiment, the concentration is 25% of saturation. In another embodiment, the concentration is 30% of saturation. In another embodiment, the concentration is 35% of saturation. In another embodiment, the concentration is 40% of saturation. In another embodiment, the concentration is 45% of saturation. In another embodiment, the concentration is 50% of saturation. In another embodiment, the concentration is 55% of saturation. In another embodiment, the concentration is 60% of saturation. In another embodiment, the concentration is 65% of saturation. In another embodiment, the concentration is 70% of saturation. In another embodiment, the concentration is 75% of saturation. In another embodiment, the concentration is 80% of saturation. In another embodiment, the concentration is 85% of saturation. In another embodiment, the concentration is 90% of saturation. In another embodiment, the concentration is 95% of saturation. In another embodiment, the concentration is 100% of saturation. In another embodiment, the concentration is near 100% of saturation. In another embodiment, the concentration is above 100% of saturation. In another embodiment, the concentration is 100-120% of saturation.

In one embodiment, the fermentation process is discontinued once an OD_{600 nm} value of 5-10 has been reached.

In another embodiment of methods and compositions disclosed herein, the culture is grown in media having a maximum volume of 2 liters (L) per vessel. In another embodiment, the media has a maximum volume of 200 ml per vessel. In another embodiment, the media has a maximum volume of 300 ml per vessel. In another embodiment, the media has a maximum volume of 500 ml per vessel. In another embodiment, the media has a maximum volume of 750 ml per vessel. In another embodiment, the media has a maximum volume of 1 L per vessel. In another embodiment, the media has a maximum volume of 1.5 L per vessel. In another embodiment, the media has a maximum volume of 2.5 L per vessel. In another embodiment, the media has a maximum volume of 3 L per vessel.

In another embodiment, the media has a minimum volume of 2 L per vessel. In another embodiment, the media has a minimum volume of 500 ml per vessel. In another embodiment, the media has a minimum volume of 750 ml per vessel. In another embodiment, the media has a minimum volume of 1 L per vessel. In another embodiment, the media has a minimum volume of 1.5 L per vessel. In another embodiment, the media has a minimum volume of 2.5 L per vessel. In another embodiment, the media has a minimum volume of 2 L per vessel. In another embodiment, the media has a minimum volume of 3 L per vessel. In another embodiment, the media has a minimum volume of 4 L per vessel. In another embodiment, the media has a minimum volume of 5 L per vessel. In another embodiment, the media has a minimum volume of 6 L per vessel. In another embodiment, the media has a minimum volume of 8 L per vessel. In another embodiment, the media has a minimum volume of 10 L per vessel. In one embodiment, the recombinant Listeria culture is grown in a fermenter system disclosed herein and is then concentrated using a filtration system. In another embodiment, no concentration is needed. In another embodiment, the filtration system is a cross flow filtration (CFF) system. In another embodiment, the filtration system is a Tangential Flow Filtration (TFF) system. In another embodiment, the fermenter system is aseptically connected to the inlet of the TFF system. In another embodiment, the fermenter system is aseptically connected to the inlet of the CFF system. In another embodiment, the CFF or TFF system is disposable.

In one embodiment, the desired weight to which the drug substance is concentrated following connecting said fermenter system to the filtration system is about 1 kg. In one embodiment, the desired weight to which the drug substance is concentrated following connecting said fermenter system to the filtration system is about 0.01 kg to 0.1 kg. In one embodiment, the desired weight to which the drug substance is concentrated following connecting said fermenter system to the filtration system is about 0.1 kg to 1 kg. In one embodiment, the desired weight to which the drug substance is concentrated following connecting said fermenter system to the filtration system is about 1 kg-5 kg. In one embodiment, the desired weight to which the drug substance is concentrated following connecting said fermenter system to the filtration system is about 5 kg-10 kg.

In another embodiment, the drug substance is concentrated 2-10 fold. In another embodiment, the drug substance is concentrated 2, 3, 4, 5, 6, 7, 8, 9 or 10 fold.

In another embodiment, the step of obtaining a retentate solution comprising a drug substance disclosed herein following filtration using TFF is further followed by diafiltering said solution comprising the drug substance using formulation buffer. In another embodiment, said diafiltering step is followed by measuring the OD of said formulation buffer solution comprising said drug substance. In another embodiment, said drug substance is diafiltered about 1-10 times (against 1-10 volumes of formulation buffer) and then transferred to an intermediate bag prior to measuring said OD.

In another embodiment, said measuring of OD is carried out following aseptically obtaining a sample from said intermediate bag.

In one embodiment, following dilution of a drug substance disclosed herein using formulation buffer to form a bulk drug substance (BDS) (step e. in the manufacturing process disclosed herein), said BDS is transferred in 1L portions and stored into 10 L product bags. In another embodiment, said BDS is transferred in 500 ml to 1 L portions into said product bag. In another embodiment, said BDS is transferred in 1 L-2 L portions into said product bag. In another embodiment, said BDS is transferred in 2 L-3 L portions into said product bag. In another embodiment, said BDS is transferred in 3 L-4 L portions into said product bag. In another embodiment, said BDS is transferred in 4 L-5 L portions into said product bag. In another embodiment, said product bags are about 5-10 L, about 10-15 L, or about 15-20 L in volume. In one embodiment, said BDS is transferred in 1 L portions into about 5×10 L product bags.

In one embodiment, the bulk drug substance is aseptically aliquoted into 1-10 mL vials from a product bag disclosed herein to make a drug product. In another embodiment, the bulk drug substance is aseptically aliquoted into 1-10 mL vials from a product bag disclosed herein to make a drug product and administering to a subject. In another embodiment, the bulk drug substance is aseptically aliquoted into about 5,000 1-10 mL vials from five 10 L product bags disclosed herein to make a drug product. In another embodiment, the bulk drug substance is aseptically aliquoted into about 2,000-5,000 1-10 mL vials from five 10 L product bags disclosed herein to make a drug product. In another embodiment, the bulk drug substance is aseptically aliquoted into about 5,000-8,000 1-10 mL vials from five 10 L product bags disclosed herein to make a drug product. In another embodiment, the bulk drug substance is aseptically aliquoted into about 8,000-10,000 1-10 mL vials from five 10 L product bags disclosed herein to make a drug product. In another embodiment, the bulk drug substance is aseptically aliquoted into 1-10 mL vials from a product bag disclosed herein to make a drug product and administering to a subject. In one embodiment, an aliquot is obtained from a bulk drug substance (BDS) manufactured by a process disclosed herein for storing. In another embodiment, an aliquot is obtained from said BDS and is stored frozen at ≤−70° C. In another embodiment, an aliquot is obtained from said BDS for quality control testing. In another embodiment, an aliquot is obtained from said BDS for testing the stability of said drug substance. In another embodiment, the 5×10 L product bags are filled with 0.5-5 kg of BDS material each.

In another embodiment of methods and compositions disclosed herein, the step of measuring, sampling, freezing or lyophilizing is performed when the culture has an OD₆₀₀ of 0.1 units. In another embodiment, the culture has an OD₆₀₀ of 0.8 units. In another embodiment, the culture has an OD₆₀₀ of 0.2 units. In another embodiment, the culture has an OD₆₀₀ of 0.3 units. In another embodiment, the culture has an OD₆₀₀ of 0.4 units. In another embodiment, the culture has an OD₆₀₀ of 0.5 units. In another embodiment, the culture has an OD₆₀₀ of 0.6 units. In another embodiment, the OD₆₀₀ is about 0.7 units. In another embodiment, the OD₆₀₀ is about 0.8 units. In another embodiment, the OD₆₀₀ is 0.6 units. In another embodiment, the OD₆₀₀ is 0.65 units. In another embodiment, the OD₆₀₀ is 0.75 units. In another embodiment, the OD₆₀₀ is 0.85 units. In another embodiment, the OD₆₀₀ is 0.9 units. In another embodiment, the OD₆₀₀ is 1 unit. In another embodiment, the OD₆₀₀ is 0.6-0.9 units. In another embodiment, the OD₆₀₀ is 0.65-0.9 units. In another embodiment, the OD₆₀₀ is 0.7-0.9 units. In another embodiment, the OD₆₀₀ is 0.75-0.9 units. In another embodiment, the OD₆₀₀ is 0.8-0.9 units. In another embodiment, the OD₆₀₀ is 0.75-1 units. In another embodiment, the OD₆₀₀ is 0.9-1 units. In another embodiment, the OD₆₀₀ is greater than 1 unit. In another embodiment, the OD₆₀₀ is significantly greater than 1 unit (e.g. when the culture is produced in a batch fermenter). In another embodiment, the OD₆₀₀ is 7.5-8.5 units. In another embodiment, the OD₆₀₀ is 1.2 units. In another embodiment, the OD₆₀₀ is 1.5 units. In another embodiment, the OD₆₀₀ is 2 units. In another embodiment, the OD₆₀₀ is 2.5 units. In another embodiment, the OD₆₀₀ is 3 units. In another embodiment, the OD₆₀₀ is 3.5 units. In another embodiment, the OD₆₀₀ is 4 units. In another embodiment, the OD₆₀₀ is 4.5 units. In another embodiment, the OD₆₀₀ is 5 units. In another embodiment, the OD₆₀₀ is 5.5 units. In another embodiment, the OD₆₀₀ is 6 units. In another embodiment, the OD₆₀₀ is 6.5 units. In another embodiment, the OD₆₀₀ is 7 units. In another embodiment, the OD₆₀₀ is 7.5 units. In another embodiment, the OD₆₀₀ is 8 units. In another embodiment, the OD₆₀₀ is 8.5 units. In another embodiment, the OD₆₀₀ is 9 units. In another embodiment, the OD₆₀₀ is 9.5 units. In another embodiment, the OD₆₀₀ is 10 units. In another embodiment, the OD₆₀₀ is more than 10 units.

In another embodiment, the OD₆₀₀ is 1-2 units. In another embodiment, the OD₆₀₀ is 1.5-2.5 units. In another embodiment, the OD₆₀₀ is 2-3 units. In another embodiment, the OD₆₀₀ is 2.5-3.5 units. In another embodiment, the OD₆₀₀ is 3-4 units. In another embodiment, the OD₆₀₀ is 3.5-4.5 units. In another embodiment, the OD₆₀₀ is 4-5 units. In another embodiment, the OD₆₀₀ is 4.5-5.5 units. In another embodiment, the OD₆₀₀ is 5-6 units. In another embodiment, the OD₆₀₀ is 5.5-6.5 units. In another embodiment, the OD₆₀₀ is 1-3 units. In another embodiment, the OD₆₀₀ is 1.5-3.5 units. In another embodiment, the OD₆₀₀ is 2-4 units. In another embodiment, the OD₆₀₀ is 2.5-4.5 units. In another embodiment, the OD₆₀₀ is 3-5 units. In another embodiment, the OD₆₀₀ is 4-6 units. In another embodiment, the OD₆₀₀ is 5-7 units. In another embodiment, the OD₆₀₀ is 2-5 units. In another embodiment, the OD₆₀₀ is 3-6 units. In another embodiment, the OD₆₀₀ is 4-7 units. In another embodiment, the OD₆₀₀ is 5-8 units. In another embodiment, the OD₆₀₀ is 1.2-7.5 units. In another embodiment, the OD₆₀₀ is 1.5-7.5 units. In another embodiment, the OD₆₀₀ is 2-7.5 units. In another embodiment, the OD₆₀₀ is 2.5-7.5 units. In another embodiment, the OD₆₀₀ is 3-7.5 units. In another embodiment, the OD₆₀₀ is 3.5-7.5 units. In another embodiment, the OD₆₀₀ is 4-7.5 units. In another embodiment, the OD₆₀₀ is 4.5-7.5 units. In another embodiment, the OD₆₀₀ is 5-7.5 units. In another embodiment, the OD₆₀₀ is 5.5-7.5 units. In another embodiment, the OD₆₀₀ is 6-7.5 units. In another embodiment, the OD₆₀₀ is 6.5-7.5 units. In another embodiment, the OD₆₀₀ is 7-7.5 units. In another embodiment, the OD₆₀₀ is more than 10 units. In another embodiment, the OD₆₀₀ is 1.2-8.5 units. In another embodiment, the OD₆₀₀ is 1.5-8.5 units. In another embodiment, the OD₆₀₀ is 2-8.5 units. In another embodiment, the OD₆₀₀ is 2.5-8.5 units. In another embodiment, the OD₆₀₀ is 3-8.5 units. In another embodiment, the OD₆₀₀ is 3.5-8.5 units. In another embodiment, the OD₆₀₀ is 4-8.5 units. In another embodiment, the OD₆₀₀ is 4.5-8.5 units. In another embodiment, the OD₆₀₀ is 5-8.5 units. In another embodiment, the OD₆₀₀ is 5.5-8.5 units. In another embodiment, the OD₆₀₀ is 6-8.5 units. In another embodiment, the OD₆₀₀ is 6.5-8.5 units. In another embodiment, the OD₆₀₀ is 7-8.5 units. In another embodiment, the OD₆₀₀ is 7.5-8.5 units. In another embodiment, the OD₆₀₀ is 8-8.5 units. In another embodiment, the OD₆₀₀ is 9.5-8.5 units. In another embodiment, the OD₆₀₀ is 10 units.

In one embodiment, an OD_{600 mm} analysis is performed to calculate the amount of Formulation Buffer that is needed to achieve a final desired OD of about 5-10 at 600 nm.

In another embodiment, the step of freezing or lyophilization is performed when the culture has a biomass of 1×10⁹ colony-forming units (CFU)/ml. In another embodiment, the biomass is 1.5×10⁹ CFU/ml. In another embodiment, the biomass is 1.5×10⁹ CFU/ml. In another embodiment, the biomass is 2×10⁹ CFU/ml. In another embodiment, the biomass is 3×10⁹ CFU/ml. In another embodiment, the biomass is 4×10⁹ CFU/ml. In another embodiment, the biomass is 5×10⁹ CFU/ml. In another embodiment, the biomass is 7×10⁹ CFU/ml. In another embodiment, the biomass is 9×10⁹ CFU/ml. In another embodiment, the biomass is 10×10⁹ CFU/ml. In another embodiment, the biomass is 12×10⁹ CFU/ml. In another embodiment, the biomass is 15×10⁹ CFU/ml. In another embodiment, the biomass is 20×10⁹ CFU/ml. In another embodiment, the biomass is 25×10⁹ CFU/ml. In another embodiment, the biomass is 30×10⁹ CFU/ml. In another embodiment, the biomass is 33×10⁹ CFU/ml. In another embodiment, the biomass is 40×10⁹ CFU/ml. In another embodiment, the biomass is 50×10⁹ CFU/ml. In another embodiment, the biomass is more than 50×10⁹ CFU/ml.

In another embodiment of methods and compositions disclosed herein, the Listeria culture is flash-frozen in liquid nitrogen, followed by storage at the final freezing temperature. In another embodiment, the culture is frozen in a more gradual manner; e.g. by placing in a vial of the culture in the final storage temperature. In another embodiment, the culture is frozen by any other method known in the art for freezing a bacterial culture.

In another embodiment of methods and compositions disclosed herein, the storage temperature of the culture is between ⁻20 and ⁻80 degrees Celsius (° C.). In another embodiment, the temperature is significantly below ⁻20° C. In another embodiment, the temperature is not warmer than ⁻70° C. In another embodiment, the temperature is ⁻70° C. In another embodiment, the temperature is about ⁻70° C. In another embodiment, the temperature is ⁻20° C. In another embodiment, the temperature is about ⁻20° C. In another embodiment, the temperature is ⁻30° C. In another embodiment, the temperature is ⁻40° C. In another embodiment, the temperature is ⁻50° C. In another embodiment, the temperature is ⁻60° C. In another embodiment, the temperature is ⁻80° C. In another embodiment, the temperature is ⁻30-⁻70° C. In another embodiment, the temperature is ⁻40-⁻70° C. In another embodiment, the temperature is ⁻50-⁻70° C. In another embodiment, the temperature is ⁻60-⁻70° C. In another embodiment, the temperature is ⁻30-⁻80° C. In another embodiment, the temperature is ⁻40-⁻80° C. In another embodiment, the temperature is ⁻50-⁻80° C. In another embodiment, the temperature is ⁻60-⁻80° C. In another embodiment, the temperature is ⁻70-⁻80° C. In another embodiment, the temperature is colder than ⁻70° C. In another embodiment, the temperature is colder than ⁻80° C.

In another embodiment of methods and compositions disclosed herein, the cryopreservation, frozen storage, or lyophilization is for a maximum of 24 hours. In another embodiment, the cryopreservation, frozen storage, or lyophilization is for maximum of 2 days. In another embodiment, the cryopreservation, frozen storage, or lyophilization is for maximum of 3 days. In another embodiment, the cryopreservation, frozen storage, or lyophilization is for maximum of 4 days. In another embodiment, the cryopreservation, frozen storage, or lyophilization is for maximum of 1 week. In another embodiment, the cryopreservation, frozen storage, or lyophilization is for maximum of 2 weeks. In another embodiment, the cryopreservation, frozen storage, or lyophilization is for maximum of 3 weeks. In another embodiment, the cryopreservation, frozen storage, or lyophilization is for maximum of 1 month. In another embodiment, the cryopreservation, frozen storage, or lyophilization is for maximum of 2 months. In another embodiment, the cryopreservation, frozen storage, or lyophilization is for maximum of 3 months. In another embodiment, the cryopreservation, frozen storage, or lyophilization is for maximum of 5 months. In another embodiment, the cryopreservation, frozen storage, or lyophilization is for maximum of 6 months. In another embodiment, the cryopreservation, frozen storage, or lyophilization is for maximum of 9 months. In another embodiment, the cryopreservation, frozen storage, or lyophilization is for maximum of 1 year.

In another embodiment, the cryopreservation, frozen storage, or lyophilization is for a minimum of 1 week. In another embodiment, the cryopreservation, frozen storage, or lyophilization is for minimum of 2 weeks. In another embodiment, the cryopreservation, frozen storage, or lyophilization is for minimum of 3 weeks. In another embodiment, the cryopreservation, frozen storage, or lyophilization is for minimum of 1 month. In another embodiment, the cryopreservation, frozen storage, or lyophilization is for minimum of 2 months. In another embodiment, the cryopreservation, frozen storage, or lyophilization is for minimum of 3 months. In another embodiment, the cryopreservation, frozen storage, or lyophilization is for minimum of 5 months. In another embodiment, the cryopreservation, frozen storage, or lyophilization is for minimum of 6 months. In another embodiment, the cryopreservation, frozen storage, or lyophilization is for minimum of 9 months. In another embodiment, the cryopreservation, frozen storage, or lyophilization is for minimum of 1 year. In another embodiment, the cryopreservation, frozen storage, or lyophilization is for minimum of 1.5 years. In another embodiment, the cryopreservation, frozen storage, or lyophilization is for minimum of 2 years. In another embodiment, the cryopreservation, frozen storage, or lyophilization is for minimum of 3 years. In another embodiment, the cryopreservation, frozen storage, or lyophilization is for minimum of 5 years. In another embodiment, the cryopreservation, frozen storage, or lyophilization is for minimum of 7 years. In another embodiment, the cryopreservation, frozen storage, or lyophilization is for minimum of 10 years. In another embodiment, the cryopreservation, frozen storage, or lyophilization is for longer than 10 years.

In another embodiment of the methods and compositions disclosed herein, the Listeria bacteria exhibit exponential growth essentially immediately after thawing following an extended period of cryopreservation or frozen storage. In another embodiment, the Listeria bacteria exhibit exponential growth essentially immediately after reconstitution following an extended period of lyophilization. In another embodiment, “essentially immediately” refers to within about 1 hour after inoculating fresh media with cells from the cell bank or starter culture. In another embodiment, the bacteria exhibit exponential growth shortly after (e.g. in various embodiments, after 10 minutes (min), 20 min, 30 min, 40 min, 50 min, 1 hour, 75 min, 90 min, 105 min, or 2 hours) thawing following the period of cryopreservation or storage.

The “extended period” of cryopreservation, frozen storage, or lyophilization is, in another embodiment, 1 month. In another embodiment, the period is 2 months. In another embodiment, the period is 3 months. In another embodiment, the period is 5 months. In another embodiment, the period is 6 months. In another embodiment, the period is 9 months. In another embodiment, the period is 1 year. In another embodiment, the period is 1.5 years. In another embodiment, the period is 2 years.

In another embodiment, “exponential growth” refers to a doubling time that is close to the maximum observed for the conditions (e.g. media type, temperature, etc.) in which the culture is growing. In another embodiment, “exponential growth” refers to a doubling time that is reasonable constant several hours (e.g. 1 hour, 1.5 hours, 2 hours, or 2.5 hours) after dilution of the culture; optionally following a brief recovery period.

In another embodiment, a Listeria vaccine strain of methods and compositions of the present invention retains a viability of over 90% after thawing following 14 days of cryopreservation. In another embodiment, the viability upon thawing is close to 100% following the period of cryopreservation. In another embodiment, the viability upon thawing is about 90%. In another embodiment, the viability upon thawing is close to 90%. In another embodiment, the viability upon thawing is at least 90%. In another embodiment, the viability upon thawing is over 80%.

In another embodiment, a Listeria vaccine strain of methods and compositions of the present invention retains a viability of over 90% after reconstitution following lyophilization. In another embodiment, the viability upon thawing is close to 100% following the period of lyophilization. In another embodiment, the viability upon thawing is about 90%. In another embodiment, the viability upon thawing is close to 90%. In another embodiment, the viability upon thawing is at least 90%. In another embodiment, the viability upon thawing is over 80%.

In another embodiment, a cell bank, frozen stock, or batch of vaccine doses of the present invention is grown in a defined microbiological media, comprising: (1) between about 0.3 and about 0.6 g/L of methionine; and (2) effective amounts of: (a) cysteine; (b) a pH buffer; (c) a carbohydrate; (d) a divalent cation; (e) ferric or ferrous ions; (f) glutamine or another nitrogen source; (g) riboflavin; (h) thioctic acid (also known as lipoic acid); (i) another or more components selected from leucine, isoleucine, valine, arginine, histidine, tryptophan, and phenylalanine; (j) one or more components selected from adenine, biotin, thiamine, pyridoxal, para-aminobenzoic acid, pantothenate, and nicotinamide; and (k) one or more components selected from cobalt, copper, boron, manganese, molybdenum, zinc, calcium, and citrate.

In another embodiment, the cell bank, frozen stock, or batch of vaccine doses is grown in a defined microbiological media, comprising: (1) between about 0.3 and about 0.6 g/L of cysteine; and (2) effective amounts of: (a) methionine; (b) a pH buffer; (c) a carbohydrate; (d) a divalent cation; (e) ferric or ferrous ions; (f) glutamine or another nitrogen source; (g) riboflavin; (h) thioctic acid; (i) one or more components selected from leucine, isoleucine, valine, arginine, histidine, tryptophan, and phenylalanine; (j) one or more components selected from adenine, biotin, thiamine, pyridoxal, para-aminobenzoic acid, pantothenate, and nicotinamide; and (k) one or more components selected from cobalt, copper, boron, manganese, molybdenum, zinc, calcium, and citrate.

In another embodiment, the cell bank, frozen stock, or batch of vaccine doses is grown in a defined microbiological media, comprising: (1) between about 0.00123-0.00246 moles of ferric or ferrous ions per liter; and (2) effective amounts of: (a) a pH buffer; (b) a carbohydrate; (c) a divalent cation; (d) methionine; (e) cysteine; (f) glutamine or another nitrogen source; (g) riboflavin; (h) thioctic acid; (i) one or more components selected from leucine, isoleucine, valine, arginine, histidine, tryptophan, and phenylalanine; (j) one or more components selected from adenine, biotin, thiamine, pyridoxal, para-aminobenzoic acid, pantothenate, and nicotinamide; and (k) one or more components selected from cobalt, copper, boron, manganese, molybdenum, zinc, calcium, and citrate.

In another embodiment, the cell bank, frozen stock, or batch of vaccine doses is grown in a defined microbiological media, comprising: (1) between about 1.8-3.6 g/L of glutamine or another nitrogen source; and (2) effective amounts of: (a) a pH buffer; (b) a carbohydrate: (c) a divalent cation; (d) methionine (e) cysteine; (f) ferric or ferrous ions (g) riboflavin (h); thioctic acid; (i) one or more components selected from leucine, isoleucine, valine, arginine, histidine, tryptophan, and phenylalanine; (j) one or more components selected from adenine, biotin, thiamine, pyridoxal, para-aminobenzoic acid, pantothenate, and nicotinamide; and (k) one or more components selected from cobalt, copper, boron, manganese, molybdenum, zinc, calcium, and citrate.

In another embodiment, the cell bank, frozen stock, or batch of vaccine doses is grown in a defined microbiological media, comprising: (1) between about 15 and about 30 mg/L of riboflavin; and (2) effective amounts of: (a) a pH buffer; (b) a carbohydrate; (c) a divalent cation; (d) methionine; (e) cysteine; (f) ferric or ferrous ions; (g) glutamine or another nitrogen source; (h) thioctic acid; (i) one or more components selected from leucine, isoleucine, valine, arginine, histidine, tryptophan, and phenylalanine; (j) one or more components selected from adenine, biotin, thiamine, pyridoxal, para-aminobenzoic acid, pantothenate, and nicotinamide; and (k) one or more components selected from cobalt, copper, boron, manganese, molybdenum, zinc, calcium, and citrate.

In another embodiment, the cell bank, frozen stock, or batch of vaccine doses is grown in a defined microbiological media, comprising (1) between about 0.3 and about 0.6 g/L of thioctic acid; and (2) effective amounts of: (a) a pH buffer; (b) a carbohydrate (c) a divalent cation; (d) methionine (e) cysteine; (f) ferric or ferrous ions; (g) glutamine or another nitrogen source; (h) riboflavin; (i) one or more components selected from leucine, isoleucine, valine, arginine, histidine, tryptophan, and phenylalanine; (j) one or more components selected from adenine, biotin, thiamine, pyridoxal, para-aminobenzoic acid, pantothenate, and nicotinamide; and (k) one or more components selected from cobalt, copper, boron, manganese, molybdenum, zinc, calcium, and citrate.

In another embodiment, the cell bank, frozen stock, or batch of vaccine doses is grown in a defined microbiological media, comprising: (1) between about 0.3 and about 0.6 g/L each of methionine and cysteine; (2) between about 0.00123 and 0.00246 moles of ferric or ferrous ions per liter; (3) between about 1.8 and about 3.6 g/L of glutamine or another nitrogen source; (4) between about 0.3 and about 0.6 g/L of thioctic acid; (5) between about 15 and about 30 mg/L of riboflavin; and (6) effective amounts of: (a) a pH buffer; (b) a carbohydrate; (c) a divalent cation; (d) one or more components selected from leucine, isoleucine, valine, arginine, histidine, tryptophan, and phenylalanine; (e) one or more components selected from adenine, biotin, thiamine, pyridoxal, para-aminobenzoic acid, pantothenate, and nicotinamide; and (f) one or more components selected from cobalt, copper, boron, manganese, molybdenum, zinc, calcium, and citrate.

In another embodiment, the cell bank, frozen stock, or batch of vaccine doses is grown in a defined microbiological media, comprising: (1) between about 0.3 and about 0.6 g/L each of methionine and cysteine; (2) between about 0.00123 and 0.00246 moles of ferric or ferrous ions per liter; (3) between about 1.8 and about 3.6 g/L of glutamine or another nitrogen source; (4) between about 0.3 and about 0.6 g/L of thioctic acid; (5) between about 15 and about 30 mg/L of riboflavin; and (6) effective amounts of: (a) a pH buffer; (b) a carbohydrate; (c) a divalent cation; (d) leucine; (e) isoleucine; (f) valine; (g) arginine; (h) histidine; (i) tryptophan; (j) phenylalanine; (k) one or more components selected from adenine, biotin, thiamine, pyridoxal, para-aminobenzoic acid, pantothenate, and nicotinamide; and (l) one or more components selected from cobalt, copper, boron, manganese, molybdenum, zinc, calcium, and citrate.

In another embodiment, the cell bank, frozen stock, or batch of vaccine doses is grown in a defined microbiological media, comprising (1) between about 0.3 and about 0.6 g/L each of one or more components selected from leucine, isoleucine, valine, arginine, histidine, tryptophan, and phenylalanine; and (2) effective amounts of: (a) a pH buffer; (b) a carbohydrate; (c) a divalent cation; (d) methionine; (e) cysteine; (f) ferric or ferrous ions; (g) glutamine or another nitrogen source; (h) riboflavin; (i) thioctic acid; (j) one or more components selected from adenine, biotin, thiamine, pyridoxal, para-aminobenzoic acid, pantothenate, and nicotinamide; and (k) one or more components selected from cobalt, copper, boron, manganese, molybdenum, zinc, calcium, and citrate.

In another embodiment, the cell bank, frozen stock, or batch of vaccine doses is grown in a defined microbiological media, comprising (1) between about 0.3 and about 0.6 g/L each of leucine, isoleucine, valine, arginine, histidine, tryptophan, and phenylalanine; and (2) effective amounts of: (a) a pH buffer; (b) a carbohydrate; (c) a divalent cation; (d) methionine; (e) cysteine; (f) ferric or ferrous ions; (g) glutamine or another nitrogen source; (h) riboflavin; (i) thioctic acid; (j) one or more components selected from adenine, biotin, thiamine, pyridoxal, para-aminobenzoic acid, pantothenate, and nicotinamide; and (k) one or more components selected from cobalt, copper, boron, manganese, molybdenum, zinc, calcium, and citrate.

In another embodiment, the cell bank, frozen stock, or batch of vaccine doses is grown in a defined microbiological media, comprising (1) between about 0.2 and about 0.75 of one or more components selected from biotin and adenine; and (2) effective amounts of: (a) a pH buffer; (b) a carbohydrate; (c) a divalent cation; (d) methionine; (e) cysteine; (f) ferric or ferrous ions; (g) glutamine or another nitrogen source; (h) riboflavin; (i) thioctic acid; (j) one or more components selected from leucine, isoleucine, valine, arginine, histidine, tryptophan, and phenylalanine; (k) one or more components selected from thiamine, pyridoxal, para-aminobenzoic acid, pantothenate, and nicotinamide; and (l) one or more components selected from cobalt, copper, boron, manganese, molybdenum, zinc, calcium, and citrate.

In another embodiment, the cell bank, frozen stock, or batch of vaccine doses is grown in a defined microbiological media, comprising (1) between about 3 and about 6 mg/L each of one or more components selected from thiamine, pyridoxal, para-aminobenzoic acid, pantothenate, and nicotinamide; and (2) effective amounts of: (a) a pH buffer; (b) a carbohydrate; (c) a divalent cation; (d) methionine; (e) cysteine; (f) ferric or ferrous ions; (g) glutamine or another nitrogen source; (h) riboflavin; (i) thioctic acid; (j) one or more components selected from leucine, isoleucine, valine, arginine, histidine, tryptophan, and phenylalanine; (k) biotin; (l) adenine; and (l) one or more components selected from cobalt, copper, boron, manganese, molybdenum, zinc, calcium, and citrate.

In another embodiment, the cell bank, frozen stock, or batch of vaccine doses is grown in a defined microbiological media, comprising: (1) between about 0.2 and about 0.75 mg/L each of one or more components selected from biotin and adenine; (2) between about 3 and about 6 mg/L each of one or more components selected from thiamine, pyridoxal, para-aminobenzoic acid, pantothenate, and nicotinamide; and (3) effective amounts of: (a) a pH buffer; (b) a carbohydrate; (c) a divalent cation; (d) methionine; (e) cysteine; (f) ferric or ferrous ions; (g) glutamine or another nitrogen source; (h) riboflavin; (i) thioctic acid; (j) one or more components selected from leucine, isoleucine, valine, arginine, histidine, tryptophan, and phenylalanine; and (k) one or more components selected from cobalt, copper, boron, manganese, molybdenum, zinc, calcium, and citrate.

In another embodiment, the cell bank, frozen stock, or batch of vaccine doses is grown in a defined microbiological media, comprising: (1) between about 0.005 and about 0.02 g/L each of one or more components selected from cobalt, copper, boron, manganese, molybdenum, zinc, and calcium; and (2) effective amounts of: (a) a pH buffer; (b) a carbohydrate; (c) a divalent cation; (d) methionine; (e) cysteine; (f) ferric or ferrous ions; (g) glutamine or another nitrogen source; (h) riboflavin; (i) thioctic acid; (j) one or more components selected from leucine, isoleucine, valine, arginine, histidine, tryptophan, and phenylalanine; and (k) one or more components selected from adenine, biotin, thiamine, pyridoxal, para-aminobenzoic acid, pantothenate, and nicotinamide.

In another embodiment, the cell bank, frozen stock, or batch of vaccine doses is grown in a defined microbiological media, comprising: (1) between about 0.4 and about 1 g/L of citrate; and (2) effective amounts of: (a) a pH buffer; (b) a carbohydrate; (c) a divalent cation; (d) methionine; (e) cysteine; (f) ferric or ferrous ions; (g) glutamine or another nitrogen source; (h) riboflavin; (i) thioctic acid; (j) one or more components selected from leucine, isoleucine, valine, arginine, histidine, tryptophan, and phenylalanine; (k) one or more components selected from cobalt, copper, boron, manganese, molybdenum, zinc, and calcium; and (l) one or more components selected from adenine, biotin, thiamine, pyridoxal, para-aminobenzoic acid, pantothenate, and nicotinamide.

In another embodiment, the cell bank, frozen stock, or batch of vaccine doses is grown in a defined microbiological media, comprising: (1) between about 0.3 and about 0.6 g/L each of methionine and cysteine; (2) between about 0.00123 and 0.00246 moles of ferric or ferrous ions per liter; (3) between about 1.8 and about 3.6 g/L of glutamine or another nitrogen source; (4) between about 0.3 and about 0.6 g/L of thioctic acid; (5) between about 15 and about 30 mg/L of riboflavin; (6) between about 0.3 and about 0.6 g/L each of one or more components selected from leucine, isoleucine, valine, arginine, histidine, tryptophan, and phenylalanine; (7) between about 0.2 and about 0.75 mg/L each of one or more components selected from biotin and adenine; (8) between about 3 and about 6 mg/L each of one or more components selected from thiamine, pyridoxal, para-aminobenzoic acid, pantothenate, and nicotinamide; (9) between about 0.005 and about 0.02 g/L each of one or more components selected from cobalt, copper, boron, manganese, molybdenum, zinc, and calcium; (10) between about 0.4 and about 1 g/L of citrate; and (11) and effective amounts of: (a) a pH buffer; (b) a carbohydrate; and (c) a divalent cation.

In another embodiment, the cell bank, frozen stock, or batch of vaccine doses is grown in a defined microbiological media, comprising: (1) between about 0.3 and about 0.6 g/L each of methionine and cysteine; (2) between about 0.00123 and 0.00246 moles of ferric or ferrous ions per liter; (3) between about 1.8 and about 3.6 g/L of glutamine or another nitrogen source; (4) between about 0.3 and about 0.6 g/L of thioctic acid; (5) between about 15 and about 30 mg/L of riboflavin; (6) between about 0.3 and about 0.6 g/L each of leucine, isoleucine, valine, arginine, histidine, tryptophan, and phenylalanine; (7) between about 0.2 and about 0.75 mg/L each of one or more components selected from biotin and adenine; (8) between about 3 and about 6 mg/L each of one or more components selected from thiamine, pyridoxal, para-aminobenzoic acid, pantothenate, and nicotinamide; (9) between about 0.005 and about 0.02 g/L each of one or more components selected from cobalt, copper, boron, manganese, molybdenum, zinc, and calcium; (10) between about 0.4 and about 1 g/L of citrate; and (11) and effective amounts of: (a) a pH buffer; (b) a carbohydrate; and (c) a divalent cation.

In another embodiment, the cell bank, frozen stock, or batch of vaccine doses is grown in a defined microbiological media, comprising: (1) between about 0.3 and about 0.6 g/L each of methionine and cysteine; (2) between about 0.00123 and 0.00246 moles of ferric or ferrous ions per liter; (3) between about 1.8 and about 3.6 g/L of glutamine or another nitrogen source; (4) between about 0.3 and about 0.6 g/L of thioctic acid; (5) between about 15 and about 30 mg/L of riboflavin; (6) between about 0.3 and about 0.6 g/L each of leucine, isoleucine, valine, arginine, histidine, tryptophan, and phenylalanine; (7) between about 0.2 and about 0.75 mg/L each of biotin and adenine; (8) between about 3 and about 6 mg/L each of thiamine, pyridoxal, para-aminobenzoic acid, pantothenate, and nicotinamide; (9) between about 0.005 and about 0.02 g/L each of one or more components selected from cobalt, copper, boron, manganese, molybdenum, zinc, and calcium; (10) between about 0.4 and about 1 g/L of citrate; and (11) and effective amounts of: (a) a pH buffer; (b) a carbohydrate; and (c) a divalent cation.

In another embodiment, a fermentation media disclosed herein comprises an aqueous solvent. In another embodiment, the aqueous solvent is water. In another embodiment, the solvent is Water for Injection (WFI). In another embodiment, the aqueous solvent is any other aqueous solvent known in the art.

In one embodiment, a fermentation media disclosed herein comprises any 2 of the components listed in Table 4. In another embodiment, a fermentation media disclosed herein comprises any 3 of the components listed in Table 4. In another embodiment, a fermentation media disclosed herein comprises any 4 of the components listed in Table 4. In another embodiment, a fermentation media disclosed herein comprises any 5 of the components listed in Table 4. In another embodiment, a fermentation media disclosed herein comprises any 6 of the components listed in Table 4. In another embodiment, a fermentation media disclosed herein comprises all of the components listed in Table 4.

In one embodiment, a buffer media disclosed herein comprises any 2 of the components listed in Table 5. In another embodiment, a buffer media disclosed herein comprises any 3 of the components listed in Table 5. In another embodiment, a buffer media disclosed herein comprises any 4 of the components listed in Table 5. In another embodiment, a buffer media disclosed herein comprises any 5 of the components listed in Table 5. In another embodiment, a buffer media disclosed herein comprises all of the components listed in Table 5.

In another embodiment, a defined microbiological media of the present invention further comprises an aqueous solvent. In another embodiment, the aqueous solvent is water. In another embodiment, the aqueous solvent is any other aqueous solvent known in the art.

The carbohydrate utilized in methods and compositions of the present invention is, in another embodiment, glucose. In another embodiment, the carbohydrate is fructose. In another embodiment, the carbohydrate is sucrose. In another embodiment, the carbohydrate is maltose. In another embodiment, the carbohydrate is lactose. In another embodiment, the carbohydrate is fructose. In another embodiment, the carbohydrate is mannose. In another embodiment, the carbohydrate is cellobiose. In another embodiment, the carbohydrate is trehalose. In another embodiment, the carbohydrate is maltose. In another embodiment, the carbohydrate is glycerol. In another embodiment, the carbohydrate is glucosamine. In another embodiment, the carbohydrate is N-acetylglucosamine. In another embodiment, the carbohydrate is N-acetylmuramic acid. In another embodiment, the carbohydrate is any other carbohydrate that can be utilized by Listeria.

In another embodiment, the amount of a carbohydrate present in a defined microbiological media of methods and compositions of the present invention is between about 12-18 grams/liter (g/L). In another embodiment, the amount is 15 g/L. In another embodiment, the amount is 10 g/L. In another embodiment, the amount is 9 g/L. In another embodiment, the amount is 11 g/L. In another embodiment, the amount is 12 g/L. In another embodiment, the amount is 13 g/L. In another embodiment, the amount is 14 g/L. In another embodiment, the amount is 16 g/L. In another embodiment, the amount is 17 g/L. In another embodiment, the amount is 18 g/L. In another embodiment, the amount is 19 g/L. In another embodiment, the amount is 20 g/L. In another embodiment, the amount is more than 20 g/L.

In another embodiment, the amount is 9-15 g/L. In another embodiment, the amount is 10-15 g/L. In another embodiment, the amount is 11-15 g/L. In another embodiment, the amount is 12-16 g/L. In another embodiment, the amount is 13-17 g/L. In another embodiment, the amount is 14-18 g/L. In another embodiment, the amount is 16-19 g/L. In another embodiment, the amount is 17-20 g/L. In another embodiment, the amount is 10-20 g/L. In another embodiment, the amount is 12-20 g/L. In another embodiment, the amount is 15-20 g/L.

In another embodiment, the total amount of carbohydrate in the media is one of the above amounts. In another embodiment, the amount of one of the carbohydrates in the media is one of the above amounts. In another embodiment, the amount of each of the carbohydrates in the media is one of the above amounts.

The cobalt present in defined microbiological media of methods and compositions of the present invention is, in another embodiment, present as a cobalt ion. In another embodiment, the cobalt is present as a cobalt salt. In another embodiment, the salt is cobalt chloride. In another embodiment, the salt is any other cobalt salt known in the art. In another embodiment, the cobalt is present as any other form of cobalt known in the art.

In another embodiment, the cobalt salt is a hydrate (e.g. cobalt chloride hexahydrate). In another embodiment, the cobalt salt is anhydrous. In another embodiment, the cobalt salt is any other form of a cobalt salt known in the art.

A hydrate of a component of a defined media of methods and compositions of the present invention is, in another embodiment, a monohydrate. In another embodiment, the hydrate is a dihydrate. In another embodiment, the hydrate is a trihydrate. In another embodiment, the hydrate is a tetrahydrate. In another embodiment, the hydrate is a pentahydrate. In another embodiment, the hydrate is a hexahydrate. In another embodiment, the hydrate is a heptahydrate. In another embodiment, the hydrate is any other hydrate known in the art.

The copper present in defined microbiological media of the methods and compositions disclosed herein is, in another embodiment, present as a copper ion. In another embodiment, the copper ion is a copper (I) ion. In another embodiment, the copper ion is a copper (II) ion. In another embodiment, the copper ion is a copper (III) ion.

In another embodiment, the copper is present as a copper salt. In another embodiment, the salt is copper chloride. In another embodiment, the salt is any other copper salt known in the art. In another embodiment, the copper is present as any other form of copper known in the art.

In another embodiment, the copper salt is a hydrate (e.g. copper chloride dihydrate). In another embodiment, the copper salt is anhydrous. In another embodiment, the copper salt is any other form of a copper salt known in the art.

The boron present in defined microbiological media of methods and compositions of the present invention is, in another embodiment, present as a borate ion. In another embodiment, the boron is present as a borate acid (e.g. boric acid, H₃BO₃). In another embodiment, the boron is present as any other form of boron known in the art.

In another embodiment, the borate salt or borate acid is a hydrate. In another embodiment, the borate salt or borate acid is anhydrous. In another embodiment, the borate salt or borate acid is any other form of a borate salt or borate acid known in the art.

The manganese present in defined microbiological media of methods and compositions of the present invention is, in another embodiment, present as a manganese ion. In another embodiment, the manganese is present as a manganese salt. In another embodiment, the salt is manganese sulfate. In another embodiment, the salt is any other manganese salt known in the art. In another embodiment, the manganese is present as any other form of manganese known in the art.

In another embodiment, the manganese salt is a hydrate (e.g. manganese sulfate monohydrate). In another embodiment, the manganese salt is anhydrous. In another embodiment, the manganese salt is any other form of a manganese salt known in the art.

The molybdenum present in defined microbiological media of methods and compositions of the present invention is, in another embodiment, present as a molybdate ion. In another embodiment, the molybdenum is present as a molybdate salt. In another embodiment, the salt is sodium molybdate. In another embodiment, the salt is any other molybdate salt known in the art. In another embodiment, the molybdenum is present as any other form of molybdenum known in the art.

In another embodiment, the molybdate salt is a hydrate (e.g. sodium molybdate dihydrate). In another embodiment, the molybdate salt is anhydrous. In another embodiment, the molybdate salt is any other form of a molybdate salt known in the art.

In one embodiment, when zinc is present in a defined microbiological media of methods and compositions of the present invention it is, in another embodiment, present as a zinc ion. In another embodiment, the zinc is present as a zinc salt. In another embodiment, the salt is zinc chloride. In another embodiment, the salt is any other zinc salt known in the art. In another embodiment, the zinc is present as any other form of zinc known in the art.

In another embodiment, the zinc salt is a hydrate (e.g. zinc chloride heptahydrate). In another embodiment, the zinc salt is anhydrous. In another embodiment, the zinc salt is any other form of a zinc salt known in the art.

In one embodiment, when iron is present in defined microbiological media of methods and compositions of the present invention it is present as a ferric ion. In another embodiment, the iron is present as a ferrous ion. In another embodiment, the iron is present as a ferric salt. In another embodiment, the iron is present as a ferrous salt. In another embodiment, the salt is ferric sulfate. In another embodiment, the salt is ferric citrate. In another embodiment, the salt is any other ferric salt known in the art. In another embodiment, the salt is any other ferrous salt known in the art. In another embodiment, the iron is present as any other form of iron known in the art.

In another embodiment, the ferric or ferrous salt is a hydrate (e.g. ferric sulfate monohydrate). In another embodiment, the ferric or ferrous salt is anhydrous. In another embodiment, the ferric or ferrous salt is any other form of a ferric or ferrous salt known in the art.

The calcium present in defined microbiological media of methods and compositions of the present invention is, in another embodiment, present as a calcium ion. In another embodiment, the calcium is present as a calcium salt. In another embodiment, the salt is calcium chloride. In another embodiment, the salt is any other calcium salt known in the art. In another embodiment, the calcium is present as any other form of calcium known in the art.

In another embodiment, the calcium salt is a hydrate (e.g. calcium chloride dihydrate). In another embodiment, the calcium salt is anhydrous. In another embodiment, the calcium salt is any other form of a calcium salt known in the art.

The citrate present in defined microbiological media of methods and compositions of the present invention is, in another embodiment, present as a citrate ion. In another embodiment, the citrate is present as a citrate salt. In another embodiment, the citrate is present as a citrate acid (e.g. citric acid). In another embodiment, the citrate is present as both ferric citrate and citric acid. In another embodiment, the citrate is present as any other form of citrate known in the art.

In another embodiment, the citrate salt or citrate acid is a hydrate. In another embodiment, the citrate salt or citrate acid is anhydrous. In another embodiment, the citrate salt or citrate acid is any other form of a citrate salt or citrate acid known in the art.

The cobalt present in defined microbiological media of methods and compositions of the present invention is, in another embodiment, present in an amount of 0.02 g/L. In another embodiment, the amount is about 0.02 g/L. In another embodiment, the amount is 0.003 g/L. In another embodiment, the amount is 0.005 g/L. In another embodiment, the amount is 0.007 g/L. In another embodiment, the amount is 0.01 g/L. In another embodiment, the amount is 0.015 g/L. In another embodiment, the amount is 0.025 g/L. In another embodiment, the amount is 0.03 g/L. In another embodiment, the amount is 0.003-0.006 g/L. In another embodiment, the amount is 0.005-0.01 g/L. In another embodiment, the amount is 0.01-0.02 g/L. In another embodiment, the amount is 0.02-0.04 g/L. In another embodiment, the amount is 0.03-0.06 g/L.

In another embodiment, the cobalt is present in an amount that is the molar equivalent of 0.02 g/L of cobalt chloride hexahydrate. In another embodiment, the amount of cobalt present is the molar equivalent of about 0.02 g/L of cobalt chloride hexahydrate. In another embodiment, the amount of cobalt present is the molar equivalent of another of the above amounts or ranges of cobalt chloride hexahydrate.

The copper present in defined microbiological media of methods and compositions of the present invention is, in another embodiment, present in an amount of 0.019 g/L. In another embodiment, the amount is about 0.019 g/L. In other embodiments, the amount is any of the amounts or ranges listed above for cobalt.

In another embodiment, the copper is present in an amount that is the molar equivalent of 0.019 g/L of copper chloride dihydrate. In another embodiment, the amount of copper present is the molar equivalent of about 0.019 g/L of copper chloride dihydrate. In another embodiment, the amount of copper present is the molar equivalent of copper chloride dihydrate in any of the amounts or ranges listed above for cobalt.

The borate present in defined microbiological media of methods and compositions of the present invention is, in another embodiment, present in an amount of 0.016 g/L. In another embodiment, the amount is about 0.016 g/L. In other embodiments, the amount is any of the amounts or ranges listed above for cobalt.

In another embodiment, the borate is present in an amount that is the molar equivalent of 0.016 g/L of boric acid. In another embodiment, the amount of borate present is the molar equivalent of about 0.016 g/L of boric acid. In another embodiment, the amount of borate present is the molar equivalent of boric acid in any of the amounts or ranges listed above for cobalt.

The manganese present in defined microbiological media of methods and compositions of the present invention is, in another embodiment, present in an amount of 0.016 g/L. In another embodiment, the amount is about 0.016 g/L. In other embodiments, the amount is any of the amounts or ranges listed above for cobalt.

In another embodiment, the manganese is present in an amount that is the molar equivalent of 0.016 g/L of manganese sulfate monohydrate. In another embodiment, the amount of manganese present is the molar equivalent of about 0.016 g/L of manganese sulfate monohydrate. In another embodiment, the amount of manganese present is the molar equivalent of manganese sulfate monohydrate in any of the amounts or ranges listed above for cobalt.

The molybdenum present in defined microbiological media of methods and compositions of the present invention is, in another embodiment, present in an amount of 0.02 g/L. In another embodiment, the amount is about 0.02 g/L. In other embodiments, the amount is any of the amounts or ranges listed above for cobalt.

In another embodiment, the molybdenum is present in an amount that is the molar equivalent of 0.2 g/L of sodium molybdate dihydrate. In another embodiment, the amount of molybdenum present is the molar equivalent of about 0.02 g/L of sodium molybdate dihydrate. In another embodiment, the amount of molybdenum present is the molar equivalent of sodium molybdate dihydrate in any of the amounts or ranges listed above for cobalt.

The zinc present in defined microbiological media of methods and compositions of the present invention is, in another embodiment, present in an amount of 0.02 g/L. In another embodiment, the amount is about 0.02 g/L. In other embodiments, the amount is any of the amounts or ranges listed above for cobalt.

In another embodiment, the zinc is present in an amount that is the molar equivalent of 0.02 g/L of zinc chloride heptahydrate. In another embodiment, the amount of zinc present is the molar equivalent of about 0.02 g/L of zinc chloride heptahydrate. In another embodiment, the amount of zinc present is the molar equivalent of zinc chloride heptahydrate in any of the amounts or ranges listed above for cobalt.

In another embodiment, ferric sulfate or a related compound is present in defined microbiological media of methods and compositions of the present invention. In another embodiment, the ferric sulfate or related compound is present in an amount of 0.01 g/L. In another embodiment, the amount is about 0.01 g/L. In other embodiments, the amount is any of the amounts or ranges listed above for cobalt.

In another embodiment, the iron is present in an amount that is the molar equivalent of 0.01 g/L of ferric sulfate. In another embodiment, the amount of iron present is the molar equivalent of about 0.01 g/L of ferric sulfate. In another embodiment, the amount of iron present is the molar equivalent of ferric sulfate in any of the amounts or ranges listed above for cobalt.

The calcium present in defined microbiological media of methods and compositions of the present invention is, in another embodiment, present in an amount of 0.01 g/L (. In another embodiment, the amount is about 0.01 g/L. In other embodiments, the amount is any of the amounts or ranges listed above for cobalt.

In another embodiment, the calcium is present in an amount that is the molar equivalent of 0.01 g/L of calcium chloride dihydrate. In another embodiment, the amount of calcium present is the molar equivalent of about 0.01 g/L of calcium chloride dihydrate. In another embodiment, the amount of calcium present is the molar equivalent of calcium chloride dihydrate in any of the amounts or ranges listed above for cobalt.

The citrate present in defined microbiological media of methods and compositions of the present invention is, in another embodiment, present in an amount of 0.9 g/L. In another embodiment, the amount is 0.6 g/L in the form of citric acid. In another embodiment, the amount is 0.4 g/L in the form of ferric citrate. In another embodiment, the amount is 0.6 g/L in the form of citric acid and 0.4 g/L in the form of ferric citrate. In another embodiment, the amount is about 0.6 g/L. In another embodiment, the amount is 0.1 g/L. In another embodiment, the amount is 0.2 g/L. In another embodiment, the amount is 0.3 g/L. In another embodiment, the amount is 0.4 g/L. In another embodiment, the amount is 0.5 g/L. In another embodiment, the amount is 0.7 g/L. In another embodiment, the amount is 0.8 g/L. In another embodiment, the amount is 1 g/L. In another embodiment, the amount is more than 1 g/L.

In another embodiment, the citrate is present in an amount that is the molar equivalent of 0.6 g/L of citric acid. In another embodiment, the amount of citrate present is the molar equivalent of about 0.6 g/L of citric acid. In another embodiment, the amount of citrate present is the molar equivalent of about 0.4 g/L of ferric citrate. In another embodiment, the amount of citrate present is the molar equivalent of 0.4 g/L of ferric citrate. In another embodiment, the amount of citrate present is the molar equivalent of 0.6 g/L of citric acid and 0.4 g/L of ferric citrate. In another embodiment, the amount of citrate present is the about molar equivalent of 0.6 g/L of citric acid and 0.4 g/L of ferric citrate. In another embodiment, the amount of citrate present is the molar equivalent of citric acid in any of the amounts or ranges listed above for citrate.

One or more of the adenine, biotin, thiamine, pyridoxal, para-aminobenzoic acid, pantothenate, and nicotinamide present in defined microbiological media of methods and compositions of the present invention are, in another embodiment, present as the free compound. In another embodiment, one of the above compounds is present as a salt thereof. In another embodiment, one of the above compounds is present as a derivative thereof. In another embodiment, one of the above compounds is present as a hydrate thereof. In other embodiments, the salt, derivative, or hydrate can be any salt, derivative, or hydrate known in the art.

The thiamine (vitamin B1) present in defined microbiological media of methods and compositions of the present invention is, in another embodiment, present in the form of thiamine HCl. In another embodiment, the thiamine is present as any other salt, derivative, or hydrate of thiamine known in the art. In another embodiment, another form of vitamin B1 is substituted for thiamine.

In another embodiment, the thiamine is present in an amount of 4 mg/L. In another embodiment, the amount is about 0.5 mg/L. In another embodiment, the amount is 0.7 mg/L. In another embodiment, the amount is 1 mg/L. In another embodiment, the amount is 1.5 mg/L. In another embodiment, the amount is 2 mg/L. In another embodiment, the amount is 3 mg/L. In another embodiment, the amount is 5 mg/L. In another embodiment, the amount is 6 mg/L. In another embodiment, the amount is 8 mg/L. In another embodiment, the amount is more than 8 mg/L. In another embodiment, the thiamine is present in an amount that is the molar equivalent of 4 mg/L of thiamine HCl. In another embodiment, the thiamine is present in an amount that is the molar equivalent of thiamine HCl in one of the above amounts.

The pyridoxal (vitamin B6) present in defined microbiological media of methods and compositions of the present invention is, in another embodiment, present in the form of pyridoxal HCl. In another embodiment, the pyridoxal is present as any other salt, derivative, or hydrate of pyridoxal known in the art. In another embodiment, another form of vitamin B6 is substituted for pyridoxal.

In another embodiment, the pyridoxal is present in an amount of 4 mg/L. In another embodiment, the amount is any of the amounts or ranges listed above for thiamine. In another embodiment, the amount of pyridoxal present is the molar equivalent of about 4 mg/L of pyridoxal HCl. In another embodiment, the amount of pyridoxal present is the molar equivalent of pyridoxal HCl in any of the amounts or ranges listed above for thiamine.

The adenine (vitamin B4) present in defined microbiological media of methods and compositions of the present invention is, in another embodiment, present in the form of free adenine. In another embodiment, the adenine is present as any other salt, derivative, or hydrate of adenine known in the art. In another embodiment, another form of vitamin B4 is substituted for adenine.

In another embodiment, the adenine is present in an amount of 0.25 mg/L. In another embodiment, the amount is any of the amounts or ranges listed above for cobalt. In another embodiment, the amount of adenine present is the molar equivalent of about 0.25 mg/L of free adenine. In another embodiment, the amount of adenine present is the molar equivalent of free adenine in any of the amounts or ranges listed above for cobalt.

The biotin (vitamin B7) present in defined microbiological media of methods and compositions of the present invention is, in another embodiment, present in the form of free biotin. In another embodiment, the biotin is present as any other salt, derivative, or hydrate of biotin known in the art. In another embodiment, another form of vitamin B7 is substituted for biotin.

In another embodiment, the biotin is present in an amount of 2 mg/L. In another embodiment, the amount is any of the amounts or ranges listed above for thiamine. In another embodiment, the amount of biotin present is the molar equivalent of about 2 mg/L of free biotin. In another embodiment, the amount of biotin present is the molar equivalent of free biotin in any of the amounts or ranges listed above for thiamine.

The para-aminobenzoic acid (vitamin B-x) present in defined microbiological media of methods and compositions of the present invention is, in another embodiment, present in the form of free para-aminobenzoic acid. In another embodiment, the para-aminobenzoic acid is present as any other salt, derivative, or hydrate of para-aminobenzoic acid known in the art. In another embodiment, another form of vitamin B-x is substituted for para-aminobenzoic acid.

In another embodiment, the para-aminobenzoic acid is present in an amount of 4 mg/L. In another embodiment, the amount is any of the amounts or ranges listed above for thiamine. In another embodiment, the amount of para-aminobenzoic acid present is the molar equivalent of about 4 mg/L of free para-aminobenzoic acid. In another embodiment, the amount of para-aminobenzoic acid present is the molar equivalent of free para-aminobenzoic acid in any of the amounts or ranges listed above for thiamine.

The pantothenate (vitamin B5) present in defined microbiological media of methods and compositions of the present invention is, in another embodiment, present in the form of calcium pantothenate. In another embodiment, the pantothenate is present as any other salt, derivative, or hydrate of pantothenate known in the art. In another embodiment, another form of vitamin B5 is substituted for pantothenate.

In another embodiment, the pantothenate is present in an amount of 4 mg/L. In another embodiment, the amount is any of the amounts or ranges listed above for thiamine. In another embodiment, the amount of pantothenate present is the molar equivalent of about 4 mg/L of calcium pantothenate. In another embodiment, the amount of pantothenate present is the molar equivalent of calcium pantothenate in any of the amounts or ranges listed above for thiamine.

The nicotinamide (vitamin B3) present in defined microbiological media of methods and compositions of the present invention is, in another embodiment, present in the form of free nicotinamide. In another embodiment, the nicotinamide is present as any other salt, derivative, or hydrate of nicotinamide known in the art. In another embodiment, another form of vitamin B3 is substituted for nicotinamide.

In another embodiment, the nicotinamide is present in an amount of 4 mg/L. In another embodiment, the amount is any of the amounts or ranges listed above for thiamine. In another embodiment, the amount of nicotinamide present is the molar equivalent of about 4 mg/L of free nicotinamide. In another embodiment, the amount of nicotinamide present is the molar equivalent of free nicotinamide in any of the amounts or ranges listed above for thiamine.

One or more of the leucine, isoleucine, valine, arginine, histidine, tryptophan, and phenylalanine present in defined microbiological media of methods and compositions of the present invention are, in another embodiment, present as free amino acids. In another embodiment, one of the above compounds is present as a salt thereof. In another embodiment, one of the above compounds is present as a derivative thereof. In another embodiment, one of the above compounds is present as a hydrate thereof. In other embodiments, the salt, derivative, or hydrate can be any salt, derivative, or hydrate known in the art. Each of the above forms of adenine, biotin, thiamine, pyridoxal, para-aminobenzoic acid, pantothenate, and nicotinamide represents a separate embodiment of the present invention.

In another embodiment, one or more of the leucine, isoleucine, valine, arginine, histidine, tryptophan, and phenylalanine is present in an amount of 0.4 g/L. In another embodiment, the amount is about 0.05 g/L. In another embodiment, the amount is 0.07 g/L. In another embodiment, the amount is 0.1 g/L. In another embodiment, the amount is 0.15 g/L. In another embodiment, the amount is 0.2 g/L. In another embodiment, the amount is 0.3 g/L. In another embodiment, the amount is 0.5 g/L. In another embodiment, the amount is 0.6 g/L. In another embodiment, the amount is 0.8 g/L. In another embodiment, the amount is more than 0.8 g/L. In another embodiment, one or more of these AA is present in an amount that is the molar equivalent of 0.4 g/L of the free AA. In another embodiment, the amount is the molar equivalent of thiamine the free AA in one of the above amounts.

In another embodiment, a defined media of methods and compositions of the present invention contains two of the amino acids (AA) selected from the following leucine, isoleucine, valine, arginine, histidine, tryptophan, and phenylalanine. In another embodiment, the defined media contains 3 of these AA. In another embodiment, the media contains 4 of these AA. In another embodiment, the media contains 3 of these AA. In another embodiment, the media contains 5 of these AA. In another embodiment, the media contains 6 of these AA. In another embodiment, the media contains all of these AA. In another embodiment, the media contains at least 2 of these AA. In another embodiment, the media contains at least 3 of these AA. In another embodiment, the media contains at least 4 of these AA. In another embodiment, the media contains at least 5 of these AA. In another embodiment, the media contains at least 6 of these AA.

In another embodiment, a defined media of methods and compositions of the present invention comprises 2 of the following vitamins adenine, biotin, thiamine, pyridoxal, para-aminobenzoic acid, pantothenate, and nicotinamide. In another embodiment, the defined media comprises 3 of these vitamins. In another embodiment, the media comprises 4 of these vitamins. In another embodiment, the media comprises 3 of these vitamins. In another embodiment, the media comprises 5 of these vitamins. In another embodiment, the media comprises 6 of these vitamins. In another embodiment, the media comprises all of these vitamins. In another embodiment, the media comprises at least 2 of these vitamins. In another embodiment, the media comprises at least 3 of these vitamins. In another embodiment, the media comprises at least 4 of these vitamins. In another embodiment, the media comprises at least 5 of these vitamins. In another embodiment, the media comprises at least 6 of these vitamins.

In another embodiment, a defined media of methods and compositions of the present invention comprises 2 of the following trace elements: cobalt, copper, boron, manganese, molybdenum, zinc, iron, calcium, and citrate. In another embodiment, the defined media comprises 3 of these trace elements. In another embodiment, the media comprises 4 of these trace elements. In another embodiment, the media comprises 3 of these trace elements. In another embodiment, the media comprises 5 of these trace elements. In another embodiment, the media comprises 6 of these trace elements. In another embodiment, the media comprises 7 of these trace elements. In another embodiment, the media comprises 7 of these trace elements. In another embodiment, the media comprises all of these trace elements. In another embodiment, the media comprises at least 2 of these trace elements. In another embodiment, the media comprises at least 3 of these trace elements. In another embodiment, the media comprises at least 4 of these trace elements. In another embodiment, the media comprises at least 5 of these trace elements. In another embodiment, the media comprises at least 6 of these trace elements. In another embodiment, the media comprises at least 7 of these trace elements. In another embodiment, the media comprises at least 8 of these trace elements.

In another embodiment, a defined media of methods and compositions of the present invention comprises more than 1 component from 2 of the above classes of components; e.g more than one of the AA listed, and more than one of the vitamins listed in the third section. In another embodiment, the media comprises more than 2 components from 2 of the above classes of components; e.g. more than 2 of the AA listed in the second section of Table 3B, and more than 2 of the trace elements listed in the fourth section. In another embodiment, the media comprises more than 3 components from 2 of the above classes. In another embodiment, the media comprises more than 4 components from 2 of the above classes. In another embodiment, the media comprises more than 5 components from 2 of the above classes. In another embodiment, the media comprises more than 6 components from 2 of the above classes. In another embodiment, the media comprises all of the components from 2 of the above classes.

In another embodiment, a media of methods and compositions of the present invention comprises more than 1 component from all of the above classes of components (e.g. more than 1 component each from AA, vitamins and trace elements). In another embodiment, the media comprises more than 2 components from all of the above classes of components. In another embodiment, the media comprises more than 3 components from all of the above classes. In another embodiment, the media comprises more than 4 components from all of the above classes. In another embodiment, the media comprises more than all components from 2 of the above classes. In another embodiment, the media comprises more than 6 components from all of the above classes. In another embodiment, the media comprises all of the components from all of the above classes.

In another embodiment, the media comprises any other combination of numbers of components from each of the above classes; e.g. 2 AA, 2 vitamins, and 3 trace elements; 3 AA, 3 vitamins, and 2 trace elements; 2 AA, 3 vitamins, and all of the trace elements, etc.

In another embodiment, a media of methods and compositions of the present invention consists of one of the above recipes, mixtures of components, lists of components in specified amounts, or combinations of numbers of components from each of the above classes.

The divalent cation present in defined microbiological media of methods and compositions of the present invention is, in another embodiment, Mg. In another embodiment, the divalent cation is Ca. In another embodiment, the divalent cation is any other divalent cation known in the art. Mg can, in other embodiments, be present in any form of Mg known in the art, e.g. MgSO₄. In another embodiment, the divalent cation is present in an amount that is the molar equivalent of about 0.41 g/mL. In other embodiments, the divalent cation is present in another effective amount, as known to those skilled in the art.

In another embodiment, a nitrogen source other than glutamine is utilized in defined media of the present invention. In another embodiment, the nitrogen source is another AA. In another embodiment, the nitrogen source is another source of peptides or proteins (e.g. casitone or casamino acids). In another embodiment, the nitrogen source is ammonium chloride. In another embodiment, the nitrogen source is ammonium nitrate. In another embodiment, the nitrogen source is ammonium sulfate. In another embodiment, the nitrogen source is another ammonium salt. In another embodiment, the nitrogen source is any other nitrogen source known in the art.

In another embodiment, a defined microbiological media of methods and compositions of the present invention does not contain a component derived from an animal source. In another embodiment, the defined microbiological media does not contain an animal-derived component of incompletely defined composition (e.g. yeast extract, bacto-tryptone, etc.).

In another embodiment, “defined microbiological media” refers to a media whose components are known. In another embodiment, the term refers to a media that does not contain a component derived from an animal source. In another embodiment, the term refers to a media whose components have been chemically characterized.

In another embodiment, a defined media of methods and compositions of the present invention supports growth of the Listeria strain to about 1.1×10¹⁰ CFU/mL (e.g. when grown in flasks;). In another embodiment, the defined media supports growth to about 1.1×10¹⁰ CFU/mL (e.g. when grown in fermenters; Examples 13-16). In another embodiment, the defined media supports growth to about 5×10⁹ CFU/mL (e.g. when grown in fermenters; Examples 13-16). In another embodiment, the defined media supports growth of viable bacteria (e.g. bacteria that can be cryopreserved without significant loss of viability) to about 3×10¹⁰ CFU/mL (e.g. when grown in fermenters; Examples 13-16). In another embodiment, the defined media supports growth to an OD₆₀₀ of about 4.5 (Examples 13-16). In other embodiments, the defined media supports growth to another OD₆₀₀ value enumerated herein. In other embodiments, the defined media supports growth to another CFU/mL value enumerated herein. In another embodiment, the defined media supports growth to a density approximately equivalent to that obtained with TB. In another embodiment, the defined media supports growth to a density approximately equivalent to that obtained with LB.

In another embodiment, a defined media of methods and compositions of the present invention supports a growth rate of the Listeria strain of about 0.25 h⁻¹ (Examples). In another embodiment, the growth rate is about 0.15 h⁻¹. In another embodiment, the growth rate is about 0.2 h⁻¹. In another embodiment, the growth rate is about 0.3 h⁻¹. In another embodiment, the growth rate is about 0.4 h⁻¹. In another embodiment, the growth rate is about 0.5 h⁻¹. In another embodiment, the growth rate is about 0.6 h⁻¹. In another embodiment, the defined media supports a growth rate approximately equivalent to that obtained with TB. In another embodiment, the defined media supports a growth rate approximately equivalent to that obtained with LB.

As provided herein, the Listeria-based immunotherapy disclosed herein were completely well tolerated in 5/6 patients, even though the patients were very sick with metastatic cancer. It should be noted that halting of therapy in the case of the other patient, Patient 5, was done purely as a precaution. At no point was the patient's life considered to be even remotely in danger. The safety results in such patients, at least some of which were likely to be immunosuppressed, shows that the Listeria vaccines can be safely administered to a wide variety of patients.

In another embodiment, a peptide of the present invention is a fusion peptide. In another embodiment, “fusion peptide” refers to a peptide or polypeptide comprising 2 or more proteins linked together by peptide bonds or other chemical bonds. In another embodiment, the proteins are linked together directly by a peptide or other chemical bond. In another embodiment, the proteins are linked together with 1 or more AA (e.g. a “spacer”) between the 2 or more proteins.

In another embodiment, a vaccine of the present invention further comprises an adjuvant. The adjuvant utilized in methods and compositions of the present invention is, in another embodiment, a granulocyte/macrophage colony-stimulating factor (GM-CSF) protein. In another embodiment, the adjuvant comprises a GM-CSF protein. In another embodiment, the adjuvant is a nucleotide molecule encoding GM-CSF. In another embodiment, the adjuvant comprises a nucleotide molecule encoding GM-CSF. In another embodiment, the adjuvant is saponin QS21. In another embodiment, the adjuvant comprises saponin QS21. In another embodiment, the adjuvant is monophosphoryl lipid A. In another embodiment, the adjuvant comprises monophosphoryl lipid A. In another embodiment, the adjuvant is SBAS2. In another embodiment, the adjuvant comprises SBAS2. In another embodiment, the adjuvant is an unmethylated CpG-containing oligonucleotide. In another embodiment, the adjuvant comprises an unmethylated CpG-containing oligonucleotide. In another embodiment, the adjuvant is an immune-stimulating cytokine. In another embodiment, the adjuvant comprises an immune-stimulating cytokine. In another embodiment, the adjuvant is a nucleotide molecule encoding an immune-stimulating cytokine. In another embodiment, the adjuvant comprises a nucleotide molecule encoding an immune-stimulating cytokine. In another embodiment, the adjuvant is or comprises a quill glycoside. In another embodiment, the adjuvant is or comprises a bacterial mitogen. In another embodiment, the adjuvant is or comprises a bacterial toxin. In another embodiment, the adjuvant is or comprises any other adjuvant known in the art.

In another embodiment, a nucleotide of the present invention is operably linked to a promoter/regulatory sequence that drives expression of the encoded peptide in the Listeria strain. Promoter/regulatory sequences useful for driving constitutive expression of a gene are well known in the art and include, but are not limited to, for example, the P_{h1yA}, P_{ActA}, and p60 promoters of Listeria, the Streptococcus bac promoter, the Streptomyces griseus sgiA promoter, and the B. thuringiensis phaZ promoter. In another embodiment, inducible and tissue specific expression of the nucleic acid encoding a peptide of the present invention is accomplished by placing the nucleic acid encoding the peptide under the control of an inducible or tissue specific promoter/regulatory sequence. Examples of tissue specific or inducible promoter/regulatory sequences which are useful for his purpose include, but are not limited to the MMTV LTR inducible promoter, and the SV40 late enhancer/promoter. In another embodiment, a promoter that is induced in response to inducing agents such as metals, glucocorticoids, and the like, is utilized. Thus, it will be appreciated that the invention includes the use of any promoter/regulatory sequence, which is either known or unknown, and which is capable of driving expression of the desired protein operably linked thereto. In one embodiment, the present invention provides a method of treating a cervical cancer in a human subject, comprising the step of administering to the subject a recombinant Listeria strain, the recombinant Listeria strain comprising a recombinant polypeptide comprising an N-terminal fragment of an LLO protein and an Human Papilloma Virus (HPV) E7 antigen, whereby the recombinant Listeria strain induces an immune response against the E7 antigen, thereby treating a cervical cancer in a human subject. In another embodiment, the recombinant Listeria strain expresses the recombinant polypeptide. In another embodiment, the recombinant Listeria strain comprises a plasmid that encodes the recombinant polypeptide. In another embodiment, the method further comprises the step of boosting the human subject with a recombinant Listeria strain of the present invention. In another embodiment, the method further comprises the step of boosting the human subject with an immunogenic composition comprising an E7 antigen. In another embodiment, the method further comprises the step of boosting the human subject with an immunogenic composition that directs a cell of the subject to express an E7 antigen.

The N-terminal LLO protein fragment and HPV E7 antigen are, in another embodiment, fused directly to one another. In another embodiment, the genes encoding the N-terminal LLO protein fragment and HPV E7 antigen are fused directly to one another. In another embodiment, the N-terminal LLO protein fragment and HPV E7 antigen are attached via a linker peptide. In another embodiment, the N-terminal LLO protein fragment and HPV E7 antigen are attached via a heterologous peptide. In another embodiment, the N-terminal LLO protein fragment is N-terminal to the HPV E7 antigen. In another embodiment, the N-terminal LLO protein fragment is the N-terminal-most portion of the fusion protein.

In another embodiment, the present invention provides a method of protecting a human subject against a cervical cancer, comprising the step of administering to the subject a recombinant Listeria strain, the recombinant Listeria strain comprising a recombinant polypeptide comprising an N-terminal fragment of an LLO protein and an HPV E7 antigen, whereby the recombinant Listeria strain induces an immune response against the E7 antigen, thereby protecting a human subject against a cervical cancer. In another embodiment, the recombinant Listeria strain expresses the recombinant polypeptide. In another embodiment, the recombinant Listeria strain comprises a plasmid that encodes the recombinant polypeptide. In another embodiment, the method further comprises the step of boosting the human subject with a recombinant Listeria strain of the present invention. In another embodiment, the method further comprises the step of boosting the human subject with an immunogenic composition comprising an E7 antigen. In another embodiment, the method further comprises the step of boosting the human subject with an immunogenic composition that directs a cell of the subject to express an E7 antigen.

In another embodiment, the present invention provides a method for inducing an immune response against a cervical cancer in a human subject, comprising the step of administering to the subject a recombinant Listeria strain, the recombinant Listeria strain comprising a recombinant polypeptide comprising an N-terminal fragment of an LLO protein and an HPV E7 antigen, thereby inducing an immune response against a cervical cancer in a human subject. In another embodiment, the recombinant Listeria strain expresses the recombinant polypeptide. In another embodiment, the recombinant Listeria strain comprises a plasmid that encodes the recombinant polypeptide. In another embodiment, the method further comprises the step of boosting the human subject with a recombinant Listeria strain of the present invention. In another embodiment, the method further comprises the step of boosting the human subject with an immunogenic composition comprising an E7 antigen. In another embodiment, the method further comprises the step of boosting the human subject with an immunogenic composition that directs a cell of the subject to express an E7 antigen.

As provided herein, recombinant Listeria strains expressing LLO-antigen fusions induce anti-tumor immunity (Example 1), elicit antigen-specific T cell proliferation (Example 2), generate antigen-specific, tumor-infiltrating T cells (Example 4), and abrogate central and peripheral tolerance to antigens such as E6 and E7 (Examples 5-9). Thus, vaccines of the present invention are efficacious at inducing immune responses against E7 and E6. Further, the recombinant Listeria strains are safe and improve disease indicators in human subjects (Example 10).

In another embodiment, the present invention provides a method of treating a cervical cancer in a human subject, comprising the step of administering to the subject a recombinant Listeria strain, the recombinant Listeria strain comprising a recombinant polypeptide comprising an N-terminal fragment of an ActA protein and an HPV E7 antigen, whereby the recombinant Listeria strain induces an immune response against the E7 antigen, thereby treating a cervical cancer in a human subject. In another embodiment, the recombinant Listeria strain expresses the recombinant polypeptide. In another embodiment, the recombinant Listeria strain comprises a plasmid that encodes the recombinant polypeptide.

In another embodiment, the present invention provides a method of protecting a human subject against a cervical cancer, comprising the step of administering to the subject a recombinant Listeria strain, the recombinant Listeria strain comprising a recombinant polypeptide comprising an N-terminal fragment of an ActA protein and an HPV E7 antigen, whereby the recombinant Listeria strain induces an immune response against the E7 antigen, thereby protecting a human subject against a cervical cancer. In another embodiment, the recombinant Listeria strain expresses the recombinant polypeptide. In another embodiment, the recombinant Listeria strain comprises a plasmid that encodes the recombinant polypeptide.

In another embodiment, the present invention provides a method for inducing an immune response against a cervical cancer in a human subject, comprising the step of administering to the subject a recombinant Listeria strain, the recombinant Listeria strain comprising a recombinant polypeptide comprising an N-terminal fragment of an ActA protein and an HPV E7 antigen, thereby inducing an immune response against a cervical cancer in a human subject. In another embodiment, the recombinant Listeria strain expresses the recombinant polypeptide. In another embodiment, the recombinant Listeria strain comprises a plasmid that encodes the recombinant polypeptide.

As provided herein, recombinant Listeria strains expressing ActA-antigen fusions induce anti-tumor immunity (Example 3), generate antigen-specific, tumor-infiltrating T cells (Example 4), and abrogate central and peripheral tolerance to antigens such as E6 and E7 (Examples 5-9). Further, recombinant Listeria strains of the present invention are safe and improve disease indicators in human subjects (Example 10).

The N-terminal ActA protein fragment and HPV E7 antigen are, in another embodiment, fused directly to one another. In another embodiment, the genes encoding the N-terminal ActA protein fragment and HPV E7 antigen are fused directly to one another. In another embodiment, the N-terminal ActA protein fragment and HPV E7 antigen are attached via a linker peptide. In another embodiment, the N-terminal ActA protein fragment and HPV E7 antigen are attached via a heterologous peptide. In another embodiment, the N-terminal ActA protein fragment is N-terminal to the HPV E7 antigen. In another embodiment, the N-terminal ActA protein fragment is the N-terminal-most portion of the fusion protein.

In another embodiment, the present invention provides a method of treating a cervical cancer in a human subject, comprising the step of administering to the subject a recombinant Listeria strain, the recombinant Listeria strain comprising a recombinant polypeptide comprising a PEST-like sequence-containing peptide and an HPV E7 antigen, whereby the recombinant Listeria strain induces an immune response against the E7 antigen, thereby treating a cervical cancer in a human subject. In another embodiment, the recombinant Listeria strain expresses the recombinant polypeptide. In another embodiment, the recombinant Listeria strain comprises a plasmid that encodes the recombinant polypeptide.

In another embodiment, the present invention provides a method of protecting a human subject against a cervical cancer, comprising the step of administering to the subject a recombinant Listeria strain, the recombinant Listeria strain comprising a recombinant polypeptide comprising a PEST-like sequence-containing peptide and an HPV E7 antigen, whereby the recombinant Listeria strain induces an immune response against the E7 antigen, thereby protecting a human subject against a cervical cancer. In another embodiment, the recombinant Listeria strain expresses the recombinant polypeptide. In another embodiment, the recombinant Listeria strain comprises a plasmid that encodes the recombinant polypeptide.

In another embodiment, the present invention provides a method for inducing an immune response against a cervical cancer in a human subject, comprising the step of administering to the subject a recombinant Listeria strain, the recombinant Listeria strain comprising a recombinant polypeptide comprising a PEST-like sequence-containing peptide and an HPV E7 antigen, thereby inducing an immune response against a cervical cancer in a human subject. In another embodiment, the recombinant Listeria strain expresses the recombinant polypeptide. In another embodiment, the recombinant Listeria strain comprises a plasmid that encodes the recombinant polypeptide.

The PEST-like sequence-containing peptide and HPV E7 antigen are, in another embodiment, fused directly to one another. In another embodiment, the genes encoding the PEST-like sequence-containing peptide and HPV E7 antigen are fused directly to one another. In another embodiment, the PEST-like sequence-containing peptide and HPV E7 antigen are attached via a linker peptide. In another embodiment, the PEST-like sequence-containing peptide and HPV E7 antigen are attached via a heterologous peptide. In another embodiment, the PEST-like sequence-containing peptide is N-terminal to the HPV E7 antigen. In another embodiment, the PEST-like sequence-containing peptide is the N-terminal-most portion of the fusion protein.

As provided herein, recombinant Listeria strains expressing PEST-like sequence-antigen fusions induce anti-tumor immunity (Example 3) and generate antigen-specific, tumor-infiltrating T cells (Example 4). Further, recombinant Listeria strains of the present invention are safe and improve disease indicators in human subjects (Example 10).

In another embodiment, the present invention provides a method for vaccinating a human subject against an HPV, comprising the step of administering to the subject a recombinant Listeria strain, the recombinant Listeria strain comprising a recombinant polypeptide comprising an N-terminal fragment of an LLO protein and an HPV E7 antigen, thereby vaccinating a human subject against an HPV. In another embodiment, the recombinant Listeria strain expresses the recombinant polypeptide. In another embodiment, the recombinant Listeria strain comprises a plasmid that encodes the recombinant polypeptide.

In another embodiment, the present invention provides a method for vaccinating a human subject against an HPV, comprising the step of administering to the subject a recombinant Listeria strain, the recombinant Listeria strain comprising a recombinant polypeptide comprising a PEST-like sequence-containing peptide and an HPV E7 antigen, thereby vaccinating a human subject against an HPV. In another embodiment, the recombinant Listeria strain expresses the recombinant polypeptide. In another embodiment, the recombinant Listeria strain comprises a plasmid that encodes the recombinant polypeptide.

In another embodiment, the present invention provides a method for vaccinating a human subject against an HPV, comprising the step of administering to the subject a recombinant Listeria strain, the recombinant Listeria strain comprising a recombinant polypeptide comprising an N-terminal fragment of an LLO protein and an HPV E7 antigen, thereby vaccinating a human subject against an HPV. In another embodiment, the recombinant Listeria strain expresses the recombinant polypeptide. In another embodiment, the recombinant Listeria strain comprises a plasmid that encodes the recombinant polypeptide.

As provided herein, recombinant Listeria strains expressing fusions of an antigen to LLO, ActA, or a PEST-like sequence-containing peptide induce anti-E6 and E7 immunity (Example 3), and abrogate central and peripheral tolerance to antigens such as E6 and E7 (Examples 5-9). Further, recombinant Listeria strains of the present invention are safe and improve disease indicators in human subjects (Example 10). Thus, Listeria strains of the present invention can be used to vaccinate a subject against an HPV, thereby preventing or inhibiting HPV-mediated carcinogenesis.

In another embodiment, the subject is at risk for developing an HPV-mediated carcinogenesis (e.g. a cervical cancer). In another embodiment, the subject is HPV-positive. In another embodiment, the subject's husband is HPV-positive. In another embodiment, the subject exhibits cervical intraepithelial neoplasia. In another embodiment, the subject exhibits a squamous intraepithelial lesion. In another embodiment, the subject exhibits a dysplasia in the cervix.

The HPV that is the target of methods of the present invention is, in another embodiment, an HPV 16. In another embodiment, the HPV is an HPV-18. In another embodiment, the HPV is selected from HPV-16 and HPV-18. In another embodiment, the HPV is an HPV-31. In another embodiment, the HPV is an HPV-35. In another embodiment, the HPV is an HPV-39. In another embodiment, the HPV is an HPV-45. In another embodiment, the HPV is an HPV-51. In another embodiment, the HPV is an HPV-52. In another embodiment, the HPV is an HPV-58. In another embodiment, the HPV is a high-risk HPV type. In another embodiment, the HPV is a mucosal HPV type.

In another embodiment, the present invention provides a method for inducing a regression of a cervical cancer in a human subject, comprising the step of administering to the subject a recombinant Listeria strain, the recombinant Listeria strain comprising a recombinant polypeptide comprising an N-terminal fragment of an LLO protein and an HPV E7 antigen, thereby inducing a regression of a cervical cancer in a human subject. In another embodiment, the recombinant Listeria strain expresses the recombinant polypeptide. In another embodiment, the recombinant Listeria strain comprises a plasmid that encodes the recombinant polypeptide.

In another embodiment, the present invention provides a method for reducing an incidence of relapse of a cervical cancer in a human subject, comprising the step of administering to the subject a recombinant Listeria strain, the recombinant Listeria strain comprising a recombinant polypeptide comprising an N-terminal fragment of an LLO protein and an HPV E7 antigen, thereby reducing an incidence of relapse of a cervical cancer in a human subject. In another embodiment, the recombinant Listeria strain expresses the recombinant polypeptide. In another embodiment, the recombinant Listeria strain comprises a plasmid that encodes the recombinant polypeptide.

In another embodiment, the present invention provides a method for suppressing a formation of a cervical tumor in a human subject, comprising the step of administering to the subject a recombinant Listeria strain, the recombinant Listeria strain comprising a recombinant polypeptide comprising an N-terminal fragment of an LLO protein and an HPV E7 antigen, thereby suppressing a formation of a cervical tumor in a human subject. In another embodiment, the recombinant Listeria strain expresses the recombinant polypeptide. In another embodiment, the recombinant Listeria strain comprises a plasmid that encodes the recombinant polypeptide.

In another embodiment, the present invention provides a method for inducing a remission of a cervical cancer in a human subject, comprising the step of administering to the subject a recombinant Listeria strain, the recombinant Listeria strain comprising a recombinant polypeptide comprising an N-terminal fragment of an LLO protein and an HPV E7 antigen, thereby inducing a remission of a cervical cancer in a human subject. In another embodiment, the recombinant Listeria strain expresses the recombinant polypeptide. In another embodiment, the recombinant Listeria strain comprises a plasmid that encodes the recombinant polypeptide.

In another embodiment, the present invention provides a method for impeding a growth of a cervical tumor in a human subject, comprising the step of administering to the subject a recombinant Listeria strain, the recombinant Listeria strain comprising a recombinant polypeptide comprising an N-terminal fragment of an LLO protein and an HPV E7 antigen, thereby impeding a growth of a cervical tumor in a human subject. In another embodiment, the recombinant Listeria strain expresses the recombinant polypeptide. In another embodiment, the recombinant Listeria strain comprises a plasmid that encodes the recombinant polypeptide.

In another embodiment, the present invention provides a method for reducing a size of a cervical tumor in a human subject, comprising the step of administering to the subject a recombinant Listeria strain, the recombinant Listeria strain comprising a recombinant polypeptide comprising an N-terminal fragment of an LLO protein and an HPV E7 antigen, thereby reducing a size of a cervical tumor in a human subject. In another embodiment, the recombinant Listeria strain expresses the recombinant polypeptide. In another embodiment, the recombinant Listeria strain comprises a plasmid that encodes the recombinant polypeptide.

The cervical tumor targeted by methods of the present invention is, in another embodiment, a squamous cell carcinoma. In another embodiment, the cervical tumor is an adenocarcinoma. In another embodiment, the cervical tumor is an adenosquamous carcinoma. In another embodiment, the cervical tumor is a small cell carcinoma. In another embodiment, the cervical tumor is any other type of cervical tumor known in the art.

In another embodiment, an HPV E6 antigen is utilized instead of or in addition to an E7 antigen in a method of the present invention for treating, protecting against, or inducing an immune response against a cervical cancer.

In another embodiment, an ActA protein fragment is utilized instead of or in addition to an LLO fragment in a method of the present invention for treating, protecting against, or inducing an immune response against a cervical cancer.

In another embodiment, a PEST-like sequence-containing protein fragment is utilized instead of or in addition to an LLO fragment in a method of the present invention for treating, protecting against, or inducing an immune response against a cervical cancer.

In another embodiment, the present invention provides a method for inducing an anti-E7 cytotoxic T cell (CTL) response in a human subject, comprising the step of administering to the subject a recombinant Listeria strain, the recombinant Listeria strain comprising a recombinant polypeptide comprising an N-terminal fragment of an LLO protein and an HPV E7 antigen, thereby inducing an anti-E7 CTL response in a human subject. In another embodiment, the recombinant Listeria strain comprises a plasmid that encodes the recombinant polypeptide. In another embodiment, the method further comprises the step of boosting the subject with a recombinant Listeria strain of the present invention. In another embodiment, the method further comprises the step of boosting the subject with an immunogenic composition comprising an E7 antigen. In another embodiment, the method further comprises the step of boosting the subject with an immunogenic composition that directs a cell of the subject to express an E7 antigen. In another embodiment, the CTL response is capable of therapeutic efficacy against an HPV-mediated disease, disorder, or symptom. In another embodiment, the CTL response is capable of prophylactic efficacy against an HPV-mediated disease, disorder, or symptom.

In another embodiment, the present invention provides a method of treating or ameliorating an HPV-mediated disease, disorder, or symptom in a subject, comprising the step of administering to the subject a recombinant Listeria strain, the recombinant Listeria strain comprising a recombinant polypeptide comprising an N-terminal fragment of an LLO protein and an HPV E7 antigen, whereby the recombinant Listeria strain induces an immune response against the E7 antigen, thereby treating or ameliorating an HPV-mediated disease, disorder, or symptom in a subject. In another embodiment, the subject is a human subject. In another embodiment, the subject is any other type of subject known in the art.

The HPV causing the disease, disorder, or symptom is, in another embodiment, an HPV 16. In another embodiment, the HPV is an HPV-18. In another embodiment, the HPV is an HPV-31. In another embodiment, the HPV is an HPV-35. In another embodiment, the HPV is an HPV-39. In another embodiment, the HPV is an HPV-45. In another embodiment, the HPV is an HPV-51. In another embodiment, the HPV is an HPV-52. In another embodiment, the HPV is an HPV-58. In another embodiment, the HPV is a high-risk HPV type. In another embodiment, the HPV is a mucosal HPV type.

In another embodiment, the HPV-mediated disease, disorder, or symptom is genital warts. In another embodiment, the HPV-mediated disease, disorder, or symptom is non-genital warts. In another embodiment, the HPV-mediated disease, disorder, or symptom is a respiratory papilloma. In another embodiment, the HPV-mediated disease, disorder, or symptom is any other HPV-mediated disease, disorder, or symptom known in the art.

In another embodiment, an HPV E6 antigen is utilized instead of or in addition to an E7 antigen in a method of the present invention for treating or ameliorating an HPV-mediated disease, disorder, or symptom.

In another embodiment, an ActA protein fragment is utilized instead of or in addition to an LLO fragment in a method of the present invention for treating or ameliorating an HPV-mediated disease, disorder, or symptom.

In another embodiment, a PEST-like sequence-containing protein fragment is utilized instead of or in addition to an LLO fragment in a method of the present invention for treating or ameliorating an HPV-mediated disease, disorder, or symptom.

In another embodiment, an HPV E6 antigen is utilized instead of or in addition to an E7 antigen in a method of the present invention for treating or ameliorating an HPV-mediated disease, disorder, or symptom.

The antigen of methods and compositions of the present invention is, in another embodiment, an HPV E7 protein. In another embodiment, the antigen is an HPV E6 protein. In another embodiment, the antigen is any other HPV protein known in the art.

“E7 antigen” refers, in another embodiment, to an E7 protein. In another embodiment, the term refers to an E7 fragment. In another embodiment, the term refers to an E7 peptide. In another embodiment, the term refers to any other type of E7 antigen known in the art.

The E7 protein of methods and compositions of the present invention is, in another embodiment, an HPV 16 E7 protein. In another embodiment, the E7 protein is an HPV-18 E7 protein. In another embodiment, the E7 protein is an HPV-31 E7 protein. In another embodiment, the E7 protein is an HPV-35 E7 protein. In another embodiment, the E7 protein is an HPV-39 E7 protein. In another embodiment, the E7 protein is an HPV-45 E7 protein. In another embodiment, the E7 protein is an HPV-51 E7 protein. In another embodiment, the E7 protein is an HPV-52 E7 protein. In another embodiment, the E7 protein is an HPV-58 E7 protein. In another embodiment, the E7 protein is an E7 protein of a high-risk HPV type. In another embodiment, the E7 protein is an E7 protein of a mucosal HPV type.

“E6 antigen” refers, in another embodiment, to an E6 protein. In another embodiment, the term refers to an E6 fragment. In another embodiment, the term refers to an E6 peptide. In another embodiment, the term refers to any other type of E6 antigen known in the art.

The E6 protein of methods and compositions of the present invention is, in another embodiment, an HPV 16 E6 protein. In another embodiment, the E6 protein is an HPV-18 E6 protein. In another embodiment, the E6 protein is an HPV-31 E6 protein. In another embodiment, the E6 protein is an HPV-35 E6 protein. In another embodiment, the E6 protein is an HPV-39 E6 protein. In another embodiment, the E6 protein is an HPV-45 E6 protein. In another embodiment, the E6 protein is an HPV-51 E6 protein. In another embodiment, the E6 protein is an HPV-52 E6 protein. In another embodiment, the E6 protein is an HPV-58 E6 protein. In another embodiment, the E6 protein is an E6 protein of a high-risk HPV type. In another embodiment, the E6 protein is an E6 protein of a mucosal HPV type.

In another embodiment, the present invention provides a method of vaccinating a human subject against an antigen of interest, the method comprising the step of administering intravenously to the human subject a recombinant Listeria strain comprising or expressing the antigen of interest, wherein the first peptide is selected from (a) an N-terminal fragment of an LLO protein; (b) an ActA protein or N-terminal fragment thereof; and (c) a PEST-like sequence-containing peptide, thereby vaccinating a human subject against an antigen of interest.

In another embodiment, the present invention provides a method of vaccinating a human subject against an antigen of interest, the method comprising the step of administering intravenously to the human subject an immunogenic composition, comprising a fusion of a first peptide to the antigen of interest, wherein the first peptide is selected from (a) an N-terminal fragment of an LLO protein; (b) an ActA protein or N-terminal fragment thereof; and (c) a PEST-like sequence-containing peptide, thereby vaccinating a human subject against an antigen of interest.

In another embodiment, the present invention provides a method of vaccinating a human subject against an antigen of interest, the method comprising the step of administering intravenously to the human subject a recombinant Listeria strain comprising a recombinant polypeptide, the recombinant polypeptide comprising a first peptide fused to the antigen of interest, wherein the first peptide is selected from (a) an N-terminal fragment of an LLO protein; (b) an ActA protein or N-terminal fragment thereof; and (c) a PEST-like sequence-containing peptide, thereby vaccinating a human subject against an antigen of interest.

In another embodiment, the present invention provides a method of inducing a CTL response in a human subject against an antigen of interest, the method comprising the step of administering to the human subject a recombinant Listeria strain comprising or expressing the antigen of interest, thereby inducing a CTL response in a human subject against an antigen of interest. In another embodiment, the step of administering is intravenous administration.

As provided herein, recombinant Listeria strains expressing LLO-antigen fusions induce anti-tumor immunity (Example 1), elicit antigen-specific T cell proliferation (Example 2), generate antigen-specific, tumor-infiltrating T cells (Example 4), and abrogate peripheral tolerance to antigens such as E6 and E7 (Examples 5-9). Thus, vaccines of the present invention are efficacious at inducing immune responses against E7 and E6. Further, the recombinant Listeria strains are safe and improve disease indicators in human subjects (Example 10).

In another embodiment, the antigen of interest is HPV-E7. In another embodiment, the antigen is HPV-E6. In another embodiment, the antigen is Her-2. In another embodiment, the antigen is NY-ESO-1. In another embodiment, the antigen is telomerase. In another embodiment, the antigen is SCCE. In another embodiment, the antigen is HMW-MAA. In another embodiment, the antigen is WT-1. In another embodiment, the antigen is HIV-1 Gag. In another embodiment, the antigen is Proteinase 3. In another embodiment, the antigen is Tyrosinase related protein 2. In another embodiment, the antigen is PSA (prostate-specific antigen). In another embodiment, the antigen is selected from E7, E6, Her-2, NY-ESO-1, telomerase, SCCE, HMW-MAA, WT-1, HIV-1 Gag, Proteinase 3, Tyrosinase related protein 2, PSA (prostate-specific antigen). In another embodiment, the antigen is a tumor-associated antigen. In another embodiment, the antigen is an infectious disease antigen.

In other embodiments, the antigen is derived from a fungal pathogen, bacteria, parasite, helminth, or viruses. In other embodiments, the antigen is selected from tetanus toxoid, hemagglutinin molecules from influenza virus, diphtheria toxoid, HIV gp120, HIV gag protein, IgA protease, insulin peptide B, Spongospora subterranea antigen, vibriose antigens, Salmonella antigens, pneumococcus antigens, respiratory syncytial virus antigens, Haemophilus influenza outer membrane proteins, Helicobacter pylori urease, Neisseria meningitidis pilins, N. gonorrhoeae pilins, the melanoma-associated antigens (TRP-2, MAGE-1, MAGE-3, gp-100, tyrosinase, MART-1, HSP-70, beta-HCG), human papilloma virus antigens E1 and E2 from type HPV-16, -18, -31, -33, -35 or -45 human papilloma viruses, the tumor antigens CEA, the ras protein, mutated or otherwise, the p53 protein, mutated or otherwise, Mucl, or pSA.

In other embodiments, the antigen is associated with one of the following diseases; cholera, diphtheria, Haemophilus, hepatitis A, hepatitis B, influenza, measles, meningitis, mumps, pertussis, small pox, pneumococcal pneumonia, polio, rabies, rubella, tetanus, tuberculosis, typhoid, Varicella-zoster, whooping cough3 yellow fever, the immunogens and antigens from Addison's disease, allergies, anaphylaxis, Bruton's syndrome, cancer, including solid and blood borne tumors, eczema, Hashimoto's thyroiditis, polymyositis, dermatomyositis, type 1 diabetes mellitus, acquired immune deficiency syndrome, transplant rejection, such as kidney, heart, pancreas, lung, bone, and liver transplants, Graves' disease, polyendocrine autoimmune disease, hepatitis, microscopic polyarteritis, polyarteritis nodosa, pemphigus, primary biliary cirrhosis, pernicious anemia, coeliac disease, antibody-mediated nephritis, glomerulonephritis, rheumatic diseases, systemic lupus erthematosus, rheumatoid arthritis, seronegative spondylarthritides, rhinitis, sjogren's syndrome, systemic sclerosis, sclerosing cholangitis, Wegener's granulomatosis, dermatitis herpetiformis, psoriasis, vitiligo, multiple sclerosis, encephalomyelitis, Guillain-Barre syndrome, myasthenia gravis, Lambert-Eaton syndrome, sclera, episclera, uveitis, chronic mucocutaneous candidiasis, urticaria, transient hypogammaglobulinemia of infancy, myeloma, X-linked hyper IgM syndrome, Wiskott-Aldrich syndrome, ataxia telangiectasia, autoimmune hemolytic anemia, autoimmune thrombocytopenia, autoimmune neutropenia, Waldenstrom's macroglobulinemia, amyloidosis, chronic lymphocytic leukemia, non-Hodgkin's lymphoma, malarial circumsporozite protein, microbial antigens, viral antigens, autoantigens, and listeriosis.

In other embodiments, the antigen is 1 of the following tumor antigens: a MAGE (Melanoma-Associated Antigen E) protein, e.g. MAGE 1, MAGE 2, MAGE 3, MAGE 4, a tyrosinase; a mutant ras protein; a mutant p53 protein; p97 melanoma antigen, a ras peptide or p53 peptide associated with advanced cancers; the HPV 16/18 antigens associated with cervical cancers, KLH antigen associated with breast carcinoma, CEA (carcinoembryonic antigen) associated with colorectal cancer, gp100, a MART1 antigen associated with melanoma, or the PSA antigen associated with prostate cancer.

The immune response induced by methods and compositions of the present invention is, in another embodiment, a T cell response. In another embodiment, the immune response comprises a T cell response. In another embodiment, the response is a CD8⁺ T cell response. In another embodiment, the response comprises a CD8⁺ T cell response.

The N-terminal LLO protein fragment of methods and compositions of the present invention comprises, in another embodiment, SEQ ID No: 6. In another embodiment, the fragment comprises an LLO signal peptide. In another embodiment, the fragment comprises SEQ ID No: 2. In another embodiment, the fragment consists approximately of SEQ ID No: 2. In another embodiment, the fragment consists essentially of SEQ ID No: 2. In another embodiment, the fragment corresponds to SEQ ID No: 2. In another embodiment, the fragment is homologous to SEQ ID No: 2. In another embodiment, the fragment is homologous to a fragment of SEQ ID No: 2. The ΔLLO used in some of the Examples was 416 AA long (exclusive of the signal sequence), as 88 residues from the amino terminus which is inclusive of the activation domain containing cysteine 484 were truncated. It will be clear to those skilled in the art that any ΔLLO without the activation domain, and in particular without cysteine 484, are suitable for methods and compositions of the present invention. In another embodiment, fusion of an E7 or E6 antigen to any ΔLLO, including the PEST-like AA sequence, SEQ ID NO: 6, enhances cell mediated and anti-tumor immunity of the antigen.

The LLO protein utilized to construct vaccines of the present invention has, in another embodiment, the sequence:

MKKIMLVFITLILVSLPIAQQTEAKDASAFNKENSISSMAPPASPPASPK
TPIEKKHADEIDKYIQGLDYNKNNVLVYHGDAVTNVPPRKGYKDGNEYIV
VEKKKKSINQNNADIQVVNAISSLTYPGALVKANSELVENQPDVLPVKRD
SLTLSIDLPGMTNQDNKIVVKNATKSNVNNAVNTLVERWNEKYAQAYPNV
SAKIDYDDEMAYSESQLIAKFGTAFKAVNNSLNVNFGAISEGKMQEEVIS
FKQIYYNVNVNEPTRPSRFFGKAVTKEQLQALGVNAENPPAYISSVAYGR
QVYLKLSTNSHSTKVKAAFDAAVSGKSVSGDVELTNIIKNSSFKAVIYGG
SAKDEVQIIDGNLGDLRDILKKGATFNRETPGVPIAYTTNFLKDNELAVI
KNNSEYIETTSKAYTDGKINIDHSGGYVAQFNISWDEVNYDPEGNEIVQH
KNWSENNKSKLAHFTSSIYLPGNARNINVYAKECTGLAWEWWRTVIDDRN
LPLVKNRNISIWGTTLYPKYSNKVDNPIE (GenBank Accession
No. P13128; SEQ ID NO: 1;

nucleic acid sequence is set forth in GenBank Accession No. X15127). The first 25 AA of the proprotein corresponding to this sequence are the signal sequence and are cleaved from LLO when it is secreted by the bacterium. Thus, in this embodiment, the full length active LLO protein is 504 residues long. In another embodiment, the above LLO fragment is used as the source of the LLO fragment incorporated in a vaccine of the present invention.

In another embodiment, the N-terminal fragment of an LLO protein utilized in compositions and methods of the present invention has the sequence:

(SEQ ID NO: 2)
MKKIMLVFITLILVSLPIAQQTEAKDASAFNKENSISSVAPPASPPASPK
TPIEKKHADEIDKYIQGLDYNKNNVLVYHGDAVTNVPPRKGYKDGNEYIV
VEKKKKSINQNNADIQVVNAISSLTYPGALVKANSELVENQPDVLPVKRD
SLTLSIDLPGMTNQDNKIVVKNATKSNVNNAVNTLVERWNEKYAQAYSNV
SAKIDYDDEMAYSESQLIAKFGTAFKAVNNSLNVNFGAISEGKMQEEVIS
FKQIYYNVNVNEPTRPSRFFGKAVTKEQLQALGVNAENPPAYISSVAYGR
QVYLKLSTNSHSTKVKAAFDAAVSGKSVSGDVELTNIIKNSSFKAVIYGG
SAKDEVQIIDGNLGDLRDILKKGATFNRETPGVPIAYTTNFLKDNELAVI
KNNSEYIETTSKAYTDGKINIDHSGGYVAQFNISWDEVNYD.

In another embodiment, the LLO fragment corresponds to about AA 20-442 of an LLO protein utilized herein.

In another embodiment, the LLO fragment has the sequence:

(SEQ ID NO: 3)
MKKIMLVFITLILVSLPIAQQTEAKDASAFNKENSISSVAPPASPPASPK
TPIEKKHADEIDKYIQGLDYNKNNVLVYHGDAVTNVPPRKGYKDGNEYIV
VEKKKKSINQNNADIQVVNAISSLTYPGALVKANSELVENQPDVLPVKRD
SLTLSIDLPGMTNQDNKIVVKNATKSNVNNAVNTLVERWNEKYAQAYSNV
SAKIDYDDEMAYSESQLIAKFGTAFKAVNNSLNVNFGAISEGKMQEEVIS
FKQIYYNVNVNEPTRPSRFFGKAVTKEQLQALGVNAENPPAYISSVAYGR
QVYLKLSTNSHSTKVKAAFDAAVSGKSVSGDVELTNIIKNSSFKAVIYGG
SAKDEVQIIDGNLGDLRDILKKGATFNRETPGVPIAYTTNFLKDNELAVI
KNNSEYIETTSKAYTD.

In another embodiment, “truncated LLO” or “ΔLLO” refers to a fragment of LLO that comprises the PEST-like domain. In another embodiment, the terms refer to an LLO fragment that comprises a PEST sequence.

In another embodiment, the terms refer to an LLO fragment that does not contain the activation domain at the amino terminus and does not include cysteine 484. In another embodiment, the terms refer to an LLO fragment that is not hemolytic. In another embodiment, the LLO fragment is rendered non-hemolytic by deletion or mutation of the activation domain. In another embodiment, the LLO fragment is rendered non-hemolytic by deletion or mutation of cysteine 484. In another embodiment, the LLO fragment is rendered non-hemolytic by deletion or mutation at another location.

In another embodiment, the LLO fragment consists of about the first 441 AA of the LLO protein. In another embodiment, the LLO fragment consists of about the first 420 AA of LLO. In another embodiment, the LLO fragment is a non-hemolytic form of the LLO protein.

In another embodiment, the LLO fragment contains residues of a homologous LLO protein that correspond to one of the above AA ranges. The residue numbers need not, in another embodiment, correspond exactly with the residue numbers enumerated above; e.g. if the homologous LLO protein has an insertion or deletion, relative to an LLO protein utilized herein, then the residue numbers can be adjusted accordingly.

In another embodiment, the LLO fragment is any other LLO fragment known in the art.

In another embodiment, the recombinant Listeria strain is administered to the human subject at a dose of 1×10⁹-3.31×10¹⁰ CFU. In another embodiment, the dose is 5-500×10⁸ CFU. In another embodiment, the dose is 7-500×10⁸ CFU. In another embodiment, the dose is 10-500×10⁸ CFU. In another embodiment, the dose is 20-500×10⁸ CFU. In another embodiment, the dose is 30-500×10⁸ CFU. In another embodiment, the dose is 50-500×10⁸ CFU. In another embodiment, the dose is 70-500×10⁸ CFU. In another embodiment, the dose is 100-500×10⁸ CFU. In another embodiment, the dose is 150-500×10⁸ CFU. In another embodiment, the dose is 5-300×10⁸ CFU. In another embodiment, the dose is 5-200×10⁸ CFU. In another embodiment, the dose is 5-150×10⁸ CFU. In another embodiment, the dose is 5-100×10⁸ CFU. In another embodiment, the dose is 5-70×10⁸ CFU. In another embodiment, the dose is 5-50×10⁸ CFU. In another embodiment, the dose is 5-30×10⁸ CFU. In another embodiment, the dose is 5-20×10⁸ CFU. In another embodiment, the dose is 1-30×10⁹ CFU. In another embodiment, the dose is 1-20×10⁹ CFU. In another embodiment, the dose is 2-30×10⁹ CFU. In another embodiment, the dose is 1-10×10⁹ CFU. In another embodiment, the dose is 2-10×10⁹ CFU. In another embodiment, the dose is 3-10×10⁹ CFU. In another embodiment, the dose is 2-7×10⁹ CFU. In another embodiment, the dose is 2-5×10⁹ CFU. In another embodiment, the dose is 3-5×10⁹ CFU.

In another embodiment, the dose is 1×10⁹ organisms. In another embodiment, the dose is 1.5×10⁹ organisms. In another embodiment, the dose is 2×10⁹ organisms. In another embodiment, the dose is 3×10⁹ organisms. In another embodiment, the dose is 4×10⁹ organisms. In another embodiment, the dose is 5×10⁹ organisms. In another embodiment, the dose is 6×10⁹ organisms. In another embodiment, the dose is 7×10⁹ organisms. In another embodiment, the dose is 8×10⁹ organisms. In another embodiment, the dose is 10×10⁹ organisms. In another embodiment, the dose is 1.5×10¹⁰ organisms. In another embodiment, the dose is 2×10¹⁰ organisms. In another embodiment, the dose is 2.5×10¹⁰ organisms. In another embodiment, the dose is 3×10¹⁰ organisms. In another embodiment, the dose is 3.3×10¹⁰ organisms. In another embodiment, the dose is 4×10¹⁰ organisms. In another embodiment, the dose is 5×10¹⁰ organisms.

In another embodiment, the recombinant polypeptide of methods of the present invention is expressed by the recombinant Listeria strain. In another embodiment, the expression is mediated by a nucleotide molecule carried by the recombinant Listeria strain.

In another embodiment, the recombinant Listeria strain expresses the recombinant polypeptide by means of a plasmid that encodes the recombinant polypeptide. In another embodiment, the plasmid comprises a gene encoding a bacterial transcription factor. In another embodiment, the plasmid encodes a Listeria transcription factor. In another embodiment, the transcription factor is prfA. In another embodiment, the transcription factor is any other transcription factor known in the art.

In another embodiment, the plasmid comprises a gene encoding a metabolic enzyme. In another embodiment, the metabolic enzyme is a bacterial metabolic enzyme. In another embodiment, the metabolic enzyme is a Listerial metabolic enzyme. In another embodiment, the metabolic enzyme is an amino acid metabolism enzyme. In another embodiment, the amino acid metabolism gene is involved in a cell wall synthesis pathway. In another embodiment, the metabolic enzyme is the product of a D-amino acid aminotransferase gene (dat). In another embodiment, the metabolic enzyme is the product of an alanine racemase gene (dal). In another embodiment, the metabolic enzyme is any other metabolic enzyme known in the art.

In another embodiment, a method of present invention further comprises the step of boosting the human subject with a recombinant Listeria strain of the present invention. In another embodiment, the recombinant Listeria strain used in the booster inoculation is the same as the strain used in the initial “priming” inoculation. In another embodiment, the booster strain is different from the priming strain. In another embodiment, the same doses are used in the priming and boosting inoculations. In another embodiment, a larger dose is used in the booster. In another embodiment, a smaller dose is used in the booster.

In another embodiment, a method of the present invention further comprises the step of inoculating the human subject with an immunogenic composition comprising the E7 antigen. In another embodiment, the immunogenic composition comprises a recombinant E7 protein or fragment thereof. In another embodiment, the immunogenic composition comprises a nucleotide molecule expressing a recombinant E7 protein or fragment thereof. In another embodiment, the non-Listerial inoculation is administered after the Listerial inoculation. In another embodiment, the non-Listerial inoculation is administered before the Listerial inoculation.

“Boosting” refers, in another embodiment, to administration of an additional vaccine dose to a subject. In another embodiment of methods of the present invention, 2 boosts (or a total of 3 inoculations) are administered. In another embodiment, 3 boosts are administered. In another embodiment, 4 boosts are administered. In another embodiment, 5 boosts are administered. In another embodiment, 6 boosts are administered. In another embodiment, more than 6 boosts are administered.

In one embodiment, an antigen disclosed herein is fused to an N-terminal ActA protein. In another embodiment, an N-terminal fragment of an ActA protein utilized in methods and compositions disclosed herein has, in another embodiment, the sequence set forth in SEQ ID NO: 4:

MRAMMVVFITANCITINPDIIFAATDSEDSSLNTDEWEEEKTEEQPSEVN
TGPRYETAREVSSRDIKELEKSNKVRNTNKADLIAMLKEKAEKGPNINNN
NSEQTENAAINEEASGADRPAIQVERRHPGLPSDSAAEIKKRRKAIASSD
SELESLTYPDKPTKVNKKKVAKESVADASESDLDSSMQSADESSPQPLKA
NQQPFFPKVFKKIKDAGKWVRDKIDENPEVKKAIVDKSAGLIDQLLTKKK
SEEVNASDFPPPPTDEELRLALPETPMLLGFNAPATSEPSSFEFPPPPTD
EELRLALPETPMLLGFNAPATSEPSSFEFPPPPTEDELEIIRETASSLDS
SFTRGDLASLRNAINRHSQNFSDFPPIPTEEELNGRGGRP.

In another embodiment, the ActA fragment comprises the sequence set forth in SEQ ID NO: 4. In another embodiment, the ActA fragment is any other ActA fragment known in the art.

In another embodiment, the recombinant nucleotide encoding a fragment of an ActA protein comprises the sequence set forth in SEQ ID NO: 5:

Atgcgtgcgatgatggtggttttcattactgccaattgcattacgattaa
ccccgacataatatttgcagcgacagatagcgaagattctagtctaaaca
cagatgaatgggaagaagaaaaaacagaagagcaaccaagcgaggtaaat
acgggaccaagatacgaaactgcacgtgaagtaagttcacgtgatattaa
agaactagaaaaatcgaataaagtgagaaatacgaacaaagcagacctaa
tagcaatgttgaaagaaaaagcagaaaaaggtccaaatatcaataataac
aacagtgaacaaactgagaatgcggctataaatgaagaggcttcaggagc
cgaccgaccagctatacaagtggagcgtcgtcatccaggattgccatcgg
atagcgcagcggaaattaaaaaaagaaggaaagccatagcatcatcggat
agtgagcttgaaagccttacttatccggataaaccaacaaaagtaaataa
gaaaaaagtggcgaaagagtcagttgcggatgcttctgaaagtgacttag
attctagcatgcagtcagcagatgagtcttcaccacaacctttaaaagca
aaccaacaaccatttttccctaaagtatttaaaaaaataaaagatgcggg
gaaatgggtacgtgataaaatcgacgaaaatcctgaagtaaagaaagcga
ttgttgataaaagtgcagggttaattgaccaattattaaccaaaaagaaa
agtgaagaggtaaatgcttcggacttcccgccaccacctacggatgaaga
gttaagacttgctttgccagagacaccaatgcttcttggttttaatgctc
ctgctacatcagaaccgagctcattcgaatttccaccaccacctacggat
gaagagttaagacttgctttgccagagacgccaatgcttcttggttttaa
tgctcctgctacatcggaaccgagctcgttcgaatttccaccgcctccaa
cagaagatgaactagaaatcatccgggaaacagcatcctcgctagattct
agttttacaagaggggatttagctagtttgagaaatgctattaatcgcca
tagtcaaaatttctctgatttcccaccaatcccaacagaagaagagttga
acgggagaggcggtagacca.

In another embodiment, the recombinant nucleotide has the sequence set forth in SEQ ID NO: 5. In another embodiment, the recombinant nucleotide comprises any other sequence that encodes a fragment of an ActA protein.

In another embodiment of methods and compositions disclosed herein, a PEST-like AA sequence is fused to the E7 or E6 antigen. As provided herein, recombinant Listeria strains expressing PEST-like sequence-antigen fusions induce anti-tumor immunity (Example 3) and generate antigen-specific, tumor-infiltrating T cells (Example 4). Further, enhanced cell mediated immunity was demonstrated for fusion proteins comprising an antigen and LLO containing the PEST-like AA sequence KENSISSMAPPASPPASPKTPIEKKHADEIDK (SEQ ID NO: 6).

Thus, fusion of an antigen to other LM PEST-like sequences and PEST-like sequences derived from other prokaryotic organisms will also enhance immunogenicity of the antigen. The PEST-like AA sequence has, in another embodiment, a sequence selected from SEQ ID NO: 7-12. In another embodiment, the PEST-like sequence is a PEST-like sequence from the LM ActA protein. In another embodiment, the PEST-like sequence is KTEEQPSEVNTGPR (SEQ ID NO: 7), KASVTDTSEGDLDSSMQSADESTPQPLK (SEQ ID NO: 8), KNEEVNASDFPPPPTDEELR (SEQ ID NO: 9), or RGGIPTSEEFSSLNSGDFTDDENSETTEEEIDR (SEQ ID NO: 10). In another embodiment, the PEST-like sequence is from Streptolysin O protein of Streptococcus sp. In another embodiment, the PEST-like sequence is from Streptococcus pyogenes Streptolysin O, e.g. KQNTASTETTTTNEQPK (SEQ ID NO: 11) at AA 35-51. In another embodiment, the PEST-like sequence is from Streptococcus equisimilis Streptolysin O, e.g. KQNTANTETTTTNEQPK (SEQ ID NO: 12) at AA 38-54. In another embodiment, the PEST-like sequence is another PEST-like AA sequence derived from a prokaryotic organism. In another embodiment, the PEST-like sequence is any other PEST-like sequence known in the art.

PEST-like sequences of other prokaryotic organism can be identified in accordance with methods such as described by, for example Rechsteiner and Rogers (1996, Trends Biochem. Sci. 21:267-271) for LM. Alternatively, PEST-like AA sequences from other prokaryotic organisms can also be identified based by this method. Other prokaryotic organisms wherein PEST-like AA sequences would be expected to include, but are not limited to, other Listeria species. In another embodiment, the PEST-like sequence is embedded within the antigenic protein. Thus, in another embodiment, “fusion” refers to an antigenic protein comprising both the antigen and the PEST-like amino acid sequence either linked at one end of the antigen or embedded within the antigen.

In another embodiment, the PEST-like sequence is identified using any other method or algorithm known in the art, e.g the CaSPredictor (Garay-Malpartida H M, Occhiucci J M, Alves J, Belizario J E. Bioinformatics. 2005 June; 21 Suppl 1:i169-76). In another embodiment, the following method is used:

A PEST index is calculated for each 30-35 AA stretch by assigning a value of 1 to the amino acids Ser, Thr, Pro, Glu, Asp, Asn, or Gln. The coefficient value (CV) for each of the PEST residue is 1 and for each of the other AA (non-PEST) is 0.

In one embodiment, the terms “PEST-like sequence”, “PEST-like amino acid sequence”, “PEST amino acid sequence” and “PEST-sequence” are used interchangeably herein.

In another embodiment, the LLO protein, ActA protein, or fragment thereof disclosed herein need not be that which is set forth exactly in the sequences set forth herein, but rather other alterations, modifications, or changes can be made that retain the functional characteristics of an LLO or ActA protein fused to an antigen as set forth elsewhere herein. In another embodiment, the present invention utilizes an analog of an LLO protein, ActA protein, or fragment thereof. Analogs differ, in another embodiment, from naturally occurring proteins or peptides by conservative AA sequence differences or by modifications which do not affect sequence, or by both.

In another embodiment, either a whole E7 protein or a fragment thereof is fused to a LLO protein, ActA protein, or PEST-like sequence-containing peptide to generate a recombinant peptide of methods of the present invention. The E7 protein that is utilized (either whole or as the source of the fragments) has, in another embodiment, the sequence

MHGDTPTLHEYMLDLQPETTDLYCYEQLNDSSEEEDEIDGPAGQAEPDRAHY NIVTFCCKCDSTLRLCVQSTHVDIRTLEDLLMGTLGIVCPICSQKP (SEQ ID No: 13). In another embodiment, the E7 protein is a homologue of SEQ ID No: 13. In another embodiment, the E7 protein is a variant of SEQ ID No: 13. In another embodiment, the E7 protein is an isomer of SEQ ID No: 13. In another embodiment, the E7 protein is a fragment of SEQ ID No: 13. In another embodiment, the E7 protein is a fragment of a homologue of SEQ ID No: 13. In another embodiment, the E7 protein is a fragment of a variant of SEQ ID No: 13. In another embodiment, the E7 protein is a fragment of an isomer of SEQ ID No: 13.

In another embodiment, the sequence of the E7 protein is:

MHGPKATLQDIVLHLEPQNEIPVDLLCHEQLSDSEEENDEIDGVNHQHLPARR AEPQRHTMLCMCCKCEARIELVVESSADDLRAFQQLFLNTLSFVCPWCASQQ (SEQ ID No: 14). In another embodiment, the E6 protein is a homologue of SEQ ID No: 14. In another embodiment, the E6 protein is a variant of SEQ ID No: 14. In another embodiment, the E6 protein is an isomer of SEQ ID No: 14. In another embodiment, the E6 protein is a fragment of SEQ ID No: 14. In another embodiment, the E6 protein is a fragment of a homologue of SEQ ID No: 14. In another embodiment, the E6 protein is a fragment of a variant of SEQ ID No: 14. In another embodiment, the E6 protein is a fragment of an isomer of SEQ ID No: 14.

In another embodiment, the E7 protein has a sequence set forth in one of the following GenBank entries: M24215, NC_004500, V01116, X62843, or M14119. In another embodiment, the E7 protein is a homologue of a sequence from one of the above GenBank entries. In another embodiment, the E7 protein is a variant of a sequence from one of the above GenBank entries. In another embodiment, the E7 protein is an isomer of a sequence from one of the above GenBank entries. In another embodiment, the E7 protein is a fragment of a sequence from one of the above GenBank entries. In another embodiment, the E7 protein is a fragment of a homologue of a sequence from one of the above GenBank entries. In another embodiment, the E7 protein is a fragment of a variant of a sequence from one of the above GenBank entries. In another embodiment, the E7 protein is a fragment of an isomer of a sequence from one of the above GenBank entries.

In another embodiment, either a whole E6 protein or a fragment thereof is fused to a LLO protein, ActA protein, or PEST-like sequence-containing peptide to generate a recombinant peptide of methods of the present invention. The E6 protein that is utilized (either whole or as the source of the fragments) has, in another embodiment, the sequence

(SEQ ID No: 15)
MHQKRTAMFQDPQERPRKLPQLCTELQTTIHDIILECVYCKQQLLRREVY
DFAFRDLCIVYRDGNPYAVCDKCLKFYSKISEYRHYCYSLYGTTLEQQYN
KPLCDLLIRCINCQKPLCPEEKQRHLDKKQRFHN1RGRWTGRCMSCCRSS
RTRRETQL.

In another embodiment, the E6 protein is a homologue of SEQ ID No: 15. In another embodiment, the E6 protein is a variant of SEQ ID No: 15. In another embodiment, the E6 protein is an isomer of SEQ ID No: 15. In another embodiment, the E6 protein is a fragment of SEQ ID No: 15. In another embodiment, the E6 protein is a fragment of a homologue of SEQ ID No: 15. In another embodiment, the E6 protein is a fragment of a variant of SEQ ID No: 15. In another embodiment, the E6 protein is a fragment of an isomer of SEQ ID No: 15.

In another embodiment, the sequence of the E6 protein is:

(SEQ ID No: 16)
MARFEDPTRRPYKLPDLCTELNTSLQDIEITCVYCKTVLELTEVFEFAFK
DLFVVYRDSIPHAACHKCIDFYSRIRELRHYSDSVYGDTLEKLTNTGLYN
LLIRCLRCQKPLNPAEKLRHLNEKRRFHNIAGHYRGQCHSCCNRARQERL
QRRRETQV.

In another embodiment, In another embodiment, the E6 protein is a homologue of SEQ ID No: 16. In another embodiment, the E6 protein is a variant of SEQ ID No: 16. In another embodiment, the E6 protein is an isomer of SEQ ID No: 16. In another embodiment, the E6 protein is a fragment of SEQ ID No: 16. In another embodiment, the E6 protein is a fragment of a homologue of SEQ ID No: 16. In another embodiment, the E6 protein is a fragment of a variant of SEQ ID No: 16. In another embodiment, the E6 protein is a fragment of an isomer of SEQ ID No: 16.

In another embodiment, the E6 protein has a sequence set forth in one of the following GenBank entries: M24215, M14119, NC_004500, V01116, X62843, or M14119. In another embodiment, the E6 protein is a homologue of a sequence from one of the above GenBank entries. In another embodiment, the E6 protein is a variant of a sequence from one of the above GenBank entries. In another embodiment, the E6 protein is an isomer of a sequence from one of the above GenBank entries. In another embodiment, the E6 protein is a fragment of a sequence from one of the above GenBank entries. In another embodiment, the E6 protein is a fragment of a homologue of a sequence from one of the above GenBank entries. In another embodiment, the E6 protein is a fragment of a variant of a sequence from one of the above GenBank entries. In another embodiment, the E6 protein is a fragment of an isomer of a sequence from one of the above GenBank entries.

In another embodiment, “homology” refers to identity to an LLO sequence (e.g. to one of SEQ ID No: 1-3) of greater than 70%. In another embodiment, “homology” refers to identity to one of SEQ ID No: 1-3 of greater than 72%. In another embodiment, “homology” refers to identity to one of SEQ ID No: 1-3 of greater than 75%. In another embodiment, “homology” refers to identity to one of SEQ ID No: 1-3 of greater than 78%. In another embodiment, “homology” refers to identity to one of SEQ ID No: 1-3 of greater than 80%. In another embodiment, “homology” refers to identity to one of SEQ ID No: 1-3 of greater than 82%. In another embodiment, “homology” refers to identity to one of SEQ ID No: 1-3 of greater than 83%. In another embodiment, “homology” refers to identity to one of SEQ ID No: 1-3 of greater than 85%. In another embodiment, “homology” refers to identity to one of SEQ ID No: 1-3 of greater than 87%. In another embodiment, “homology” refers to identity to one of SEQ ID No: 1-3 of greater than 88%. In another embodiment, “homology” refers to identity to one of SEQ ID No: 1-3 of greater than 90%. In another embodiment, “homology” refers to identity to one of SEQ ID No: 1-3 of greater than 92%. In another embodiment, “homology” refers to identity to one of SEQ ID No: 1-3 of greater than 93%. In another embodiment, “homology” refers to identity to one of SEQ ID No: 1-3 of greater than 95%. In another embodiment, “homology” refers to identity to one of SEQ ID No: 1-3 of greater than 96%. In another embodiment, “homology” refers to identity to one of SEQ ID No: 1-3 of greater than 97%. In another embodiment, “homology” refers to identity to one of SEQ ID No: 1-3 of greater than 98%. In another embodiment, “homology” refers to identity to one of SEQ ID No: 1-3 of greater than 99%. In another embodiment, “homology” refers to identity to one of SEQ ID No: 1-3 of 100%.

In another embodiment, “homology” refers to identity to an E7 sequence (e.g. to one of SEQ ID No: 13-14) of greater than 70%. In another embodiment, “homology” refers to identity to one of SEQ ID No: 13-14 of greater than 72%. In another embodiment, “homology” refers to identity to one of SEQ ID No: 13-14 of greater than 75%. In another embodiment, “homology” refers to identity to one of SEQ ID No: 13-14 of greater than 78%. In another embodiment, “homology” refers to identity to one of SEQ ID No: 13-14 of greater than 80%. In another embodiment, “homology” refers to identity to one of SEQ ID No: 13-14 of greater than 82%. In another embodiment, “homology” refers to identity to one of SEQ ID No: 13-14 of greater than 83%. In another embodiment, “homology” refers to identity to one of SEQ ID No: 13-14 of greater than 85%. In another embodiment, “homology” refers to identity to one of SEQ ID No: 13-14 of greater than 87%. In another embodiment, “homology” refers to identity to one of SEQ ID No: 13-14 of greater than 88%. In another embodiment, “homology” refers to identity to one of SEQ ID No: 13-14 of greater than 90%. In another embodiment, “homology” refers to identity to one of SEQ ID No: 13-14 of greater than 92%. In another embodiment, “homology” refers to identity to one of SEQ ID No: 13-14 of greater than 93%. In another embodiment, “homology” refers to identity to one of SEQ ID No: 13-14 of greater than 95%. In another embodiment, “homology” refers to identity to one of SEQ ID No: 13-14 of greater than 96%. In another embodiment, “homology” refers to identity to one of SEQ ID No: 13-14 of greater than 97%. In another embodiment, “homology” refers to identity to one of SEQ ID No: 13-14 of greater than 98%. In another embodiment, “homology” refers to identity to one of SEQ ID No: 13-14 of greater than 99%. In another embodiment, “homology” refers to identity to one of SEQ ID No: 13-14 of 100%.

In another embodiment, “homology” refers to identity to an E6 sequence (e.g. to one of SEQ ID No: 15-16) of greater than 70%. In another embodiment, “homology” refers to identity to one of SEQ ID No: 15-16 of greater than 72%. In another embodiment, “homology” refers to identity to one of SEQ ID No: 15-16 of greater than 75%. In another embodiment, “homology” refers to identity to one of SEQ ID No: 15-16 of greater than 78%. In another embodiment, “homology” refers to identity to one of SEQ ID No: 15-16 of greater than 80%. In another embodiment, “homology” refers to identity to one of SEQ ID No: 15-16 of greater than 82%. In another embodiment, “homology” refers to identity to one of SEQ ID No: 15-16 of greater than 83%. In another embodiment, “homology” refers to identity to one of SEQ ID No: 15-16 of greater than 85%. In another embodiment, “homology” refers to identity to one of SEQ ID No: 15-16 of greater than 87%. In another embodiment, “homology” refers to identity to one of SEQ ID No: 15-16 of greater than 88%. In another embodiment, “homology” refers to identity to one of SEQ ID No: 15-16 of greater than 90%. In another embodiment, “homology” refers to identity to one of SEQ ID No: 15-16 of greater than 92%. In another embodiment, “homology” refers to identity to one of SEQ ID No: 15-16 of greater than 93%. In another embodiment, “homology” refers to identity to one of SEQ ID No: 15-16 of greater than 95%. In another embodiment, “homology” refers to identity to one of SEQ ID No: 15-16 of greater than 96%. In another embodiment, “homology” refers to identity to one of SEQ ID No: 15-16 of greater than 97%. In another embodiment, “homology” refers to identity to one of SEQ ID No: 15-16 of greater than 98%. In another embodiment, “homology” refers to identity to one of SEQ ID No: 15-16 of greater than 99%. In another embodiment, “homology” refers to identity to one of SEQ ID No: 15-16 of 100%.

In another embodiment, “homology” refers to identity to a PEST-like sequence (e.g. to one of SEQ ID No: 6-12) or to an ActA sequence (e.g. to one of SEQ ID No: 4-5) of greater than 70%. In another embodiment, “homology” refers to identity to one of SEQ ID No: 6-12 or SEQ ID No: 4-5 of greater than 72%. In another embodiment, “homology” refers to identity to one of SEQ ID No: 6-12 or SEQ ID No: 4-5 of greater than 75%. In another embodiment, “homology” refers to identity to one of SEQ ID No: 6-12 or SEQ ID No: 4-5 of greater than 78%. In another embodiment, “homology” refers to identity to one of SEQ ID No: 6-12 or SEQ ID No: 4-5 of greater than 80%. In another embodiment, “homology” refers to identity to one of SEQ ID No: 6-12 or SEQ ID No: 4-5 of greater than 82%. In another embodiment, “homology” refers to identity to one of SEQ ID No: 6-12 or SEQ ID No: 4-5 of greater than 83%. In another embodiment, “homology” refers to identity to one of SEQ ID No: 6-12 or SEQ ID No: 4-5 of greater than 85%. In another embodiment, “homology” refers to identity to one of SEQ ID No: 6-12 or SEQ ID No: 4-5 of greater than 87%. In another embodiment, “homology” refers to identity to one of SEQ ID No: 6-12 or SEQ ID No: 4-5 of greater than 88%. In another embodiment, “homology” refers to identity to one of SEQ ID No: 6-12 or SEQ ID No: 4-5 of greater than 90%. In another embodiment, “homology” refers to identity to one of SEQ ID No: 6-12 or SEQ ID No: 4-5 of greater than 92%. In another embodiment, “homology” refers to identity to one of SEQ ID No: 6-12 or SEQ ID No: 4-5 of greater than 93%. In another embodiment, “homology” refers to identity to one of SEQ ID No: 6-12 or SEQ ID No: 4-5 of greater than 95%. In another embodiment, “homology” refers to identity to one of SEQ ID No: 6-12 or SEQ ID No: 4-5 of greater than 96%. In another embodiment, “homology” refers to identity to one of SEQ ID No: 6-12 or SEQ ID No: 4-5 of greater than 97%. In another embodiment, “homology” refers to identity to one of SEQ ID No: 6-12 or SEQ ID No: 4-5 of greater than 98%. In another embodiment, “homology” refers to identity to one of SEQ ID No: 6-12 or SEQ ID No: 4-5 of greater than 99%. In another embodiment, “homology” refers to identity to one of SEQ ID No: 6-12 or SEQ ID No: 4-5 of 100%.

Protein and/or peptide homology for any AA sequence listed herein is determined, in one embodiment, by methods well described in the art, including immunoblot analysis, or via computer algorithm analysis of AA sequences, utilizing any of a number of software packages available, via established methods. Some of these packages include the FASTA, BLAST, MPsrch or Scanps packages, and employ, in other embodiments, the use of the Smith and Waterman algorithms, and/or global/local or BLOCKS alignments for analysis, for example. Each method of determining homology represents a separate embodiment of the present invention.

In another embodiment, the LLO protein, ActA protein, or fragment thereof is attached to the E7 or E6 antigen by chemical conjugation. In another embodiment, glutaraldehyde is used for the conjugation. In another embodiment, the conjugation is performed using any suitable method known in the art. Each possibility represents another embodiment of the present invention.

In another embodiment, fusion proteins of the present invention are prepared by any suitable method, including, for example, cloning and restriction of appropriate sequences or direct chemical synthesis by methods discussed below. In another embodiment, subsequences are cloned and the appropriate subsequences cleaved using appropriate restriction enzymes. The fragments are then ligated, in another embodiment, to produce the desired DNA sequence. In another embodiment, DNA encoding the fusion protein is produced using DNA amplification methods, for example polymerase chain reaction (PCR). First, the segments of the native DNA on either side of the new terminus are amplified separately. The 5′ end of the one amplified sequence encodes the peptide linker, while the 3′ end of the other amplified sequence also encodes the peptide linker. Since the 5′ end of the first fragment is complementary to the 3′ end of the second fragment, the two fragments (after partial purification, e.g. on LMP agarose) can be used as an overlapping template in a third PCR reaction. The amplified sequence will contain codons, the segment on the carboxy side of the opening site (now forming the amino sequence), the linker, and the sequence on the amino side of the opening site (now forming the carboxyl sequence). The insert is then ligated into a plasmid.

In another embodiment, the LLO protein, ActA protein, or fragment thereof and the E7, E6, or fragment thereof are conjugated by a means known to those of skill in the art. In another embodiment, the E7, E6, or fragment thereof is conjugated, either directly or through a linker (spacer), to the ActA protein or LLO protein. In another embodiment, the chimeric molecule is recombinantly expressed as a single-chain fusion protein.

In another embodiment, the present invention provides a kit comprising vaccine of the present invention, an applicator, and instructional material that describes use of the methods of the invention. Although model kits are described below, the contents of other useful kits will be apparent to the skilled artisan in light of the present disclosure. Each of these kits represents a separate embodiment of the present invention.

In one embodiment, the singular forms of words such as “a,” “an,” and “the,” include their corresponding plural references unless the context clearly dictates otherwise.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

It will be appreciated by a skilled artisan that the term “About” when used to modify a numerically defined parameter (e.g., the dose of a PD-1 antagonist or the length of treatment time with a combination therapy described herein) may encompass variation of the parameter in quantitative terms plus or minus 5%, or in another embodiment plus or minus 10%, or in another embodiment plus or minus 15%, or in another embodiment plus or minus 20% of stated numerical value for that parameter. For example, a dose of about 200 mg of the PD-1 antagonist, may vary between 180 mg and 220 mg.

It is to be understood by the skilled artisan that the term “subject” can encompass a mammal including an adult human or a human child, teenager or adolescent in need of therapy for, or susceptible to, a condition or its sequelae, and also may include non-human mammals such as dogs, cats, pigs, cows, sheep, goats, horses, rats, and mice. It will also be appreciated that the term may encompass livestock. The term “subject” does not exclude an individual that is normal in all respects.

It will be appreciated by the skilled artisan that the term “mammal” for purposes of treatment refers to any animal classified as a mammal, including, but not limited to, humans, domestic and farm animals, and zoo, sports, or pet animals, such as canines, including dogs, and horses, cats, cattle, pigs, sheep, etc.

In the following examples, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Thus these examples should in no way be construed, as limiting the broad scope of the invention.

### Experimental Details Section

### EXAMPLE 1

### LLO-Antigen Fusions Induce Anti-Tumor Immunity

### Materials And Experimental Methods (Examples 1-2)

### Cell Lines

The C57BL/6 syngeneic TC-1 tumor was immortalized with HPV-16 E6 and E7 and transformed with the c-Ha-ras oncogene. TC-1, provided by T. C. Wu (Johns Hopkins University School of Medicine, Baltimore, Md.) is a highly tumorigenic lung epithelial cell expressing low levels of with HPV-16 E6 and E7 and transformed with the c-Ha-ras oncogene. TC-1 was grown in RPMI 1640, 10% FCS, 2 mM L-glutamine, 100 U/ml penicillin, 100 μg/ml streptomycin, 100 μM nonessential amino acids, 1 mM sodium pyruvate, 50 micromolar (mcM) 2-ME, 400 microgram (mcg)/ml G418, and 10% National Collection Type Culture-109 medium at 37° with 10% CO₂. C3 is a mouse embryo cell from C57BL/6 mice immortalized with the complete genome of HPV 16 and transformed with pEJ-ras. EL-4/E7 is the thymoma EL-4 retrovirally transduced with E7.

L. Monocytogenes Strains and Propagation

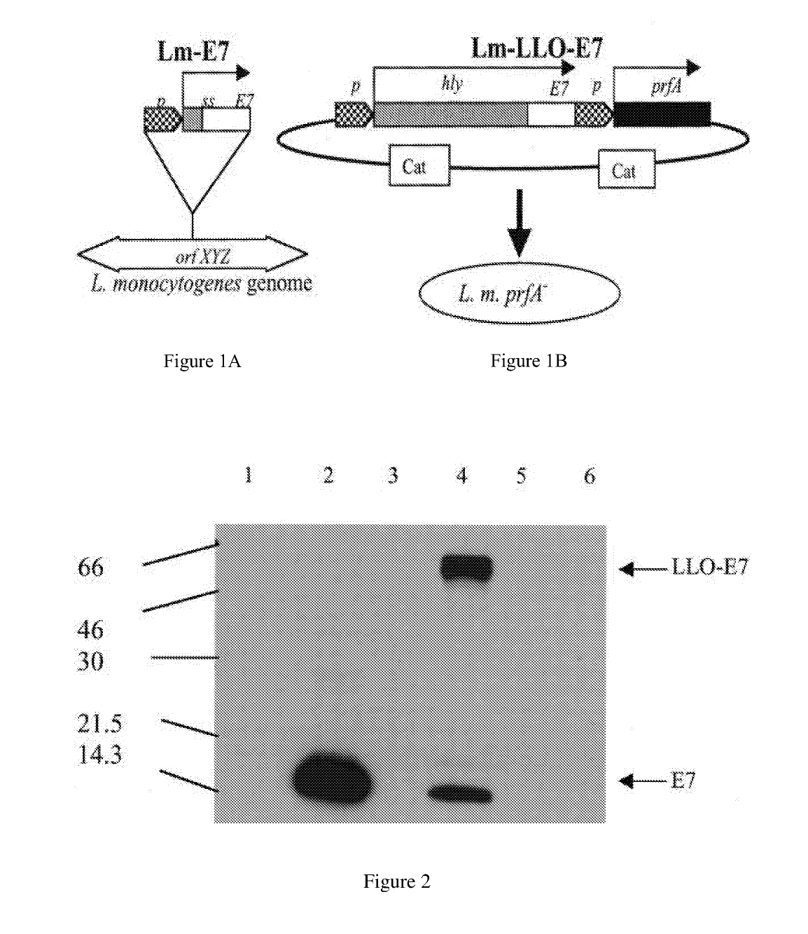

Listeria strains used were Lm-LLO-E7 (hly-E7 fusion gene in an episomal expression system; FIG. 1A), Lm-E7 (single-copy E7 gene cassette integrated into Listeria genome), Lm-LLO-NP (“DP-L2028”; hly-NP fusion gene in an episomal expression system), and Lm-Gag (“ZY-18”; single-copy HIV-1 Gag gene cassette integrated into the chromosome). E7 was amplified by PCR using the primers 5′-GGCTCGAGCATGGAGATACACC-3′ (SEQ ID No: 17; XhoI site is underlined) and 5′-GGGGACTAGTTTATGGTTTCTGAGAACA-3′ (SEQ ID No: 18; SpeI site is underlined) and ligated into pCR2.1 (Invitrogen, San Diego, Calif.). E7 was excised from pCR2.1 by XhoI/SpeI digestion and ligated into pGG-55. The hly-E7 fusion gene and the pluripotential transcription factor prfA were cloned into pAM401, a multicopy shuttle plasmid (Wirth R et al, J Bacteriol, 165: 831, 1986), generating pGG-55. The hly promoter drives the expression of the first 441 AA of the hly gene product, (lacking the hemolytic C-terminus, referred to below as “ΔLLO,” and having the sequence set forth in SEQ ID No: 2), which is joined by the XhoI site to the E7 gene, yielding a hly-E7 fusion gene that is transcribed and secreted as LLO-E7. Transformation of a prfA negative strain of Listeria, XFL-7 (provided by Dr. Hao Shen, University of Pennsylvania), with pGG-55 selected for the retention of the plasmid in vivo (FIGS. 1A-B). The hly promoter and gene fragment were generated using primers 5′-GGGGGCTAGCCCTCCTTTGATTAGTATATTC-3′ (SEQ ID No: 19; NheI site is underlined) and 5′-CTCCCTCGAGATCATAATTTACTTCATC-3′ (SEQ ID No: 20; XhoI site is underlined). The prfA gene was PCR amplified using primers 5′-GACTACAAGGACGATGACCGACAAGTGATAACCCGGGATCTAAATAAATCCGTTT-3′ (SEQ ID No: 21; XbaI site is underlined) and 5′-CCCGTCGACCAGCTCTTCTTGGTGAAG-3′ (SEQ ID No: 22; SalI site is underlined). Lm-E7 was generated by introducing an expression cassette containing the hly promoter and signal sequence driving the expression and secretion of E7 into the orfZ domain of the LM genome. E7 was amplified by PCR using the primers 5′-GCGGATCCCATGGAGATACACCTAC-3′ (SEQ ID No: 23; BamHI site is underlined) and 5′-GCTCTAGATTATGGTTTCTGAG-3′

(SEQ ID No: 24; XbaI site is underlined). E7 was then ligated into the pZY-21 shuttle vector. LM strain 10403S was transformed with the resulting plasmid, pZY-21-E7, which includes an expression cassette inserted in the middle of a 1.6-kb sequence that corresponds to the orfX, Y, Z domain of the LM genome. The homology domain allows for insertion of the E7 gene cassette into the orfZ domain by homologous recombination. Clones were screened for integration of the E7 gene cassette into the orfZ domain. Bacteria were grown in brain heart infusion medium with (Lm-LLO-E7 and Lm-LLO-NP) or without (Lm-E7 and ZY-18) chloramphenicol (20 μg/ml). Bacteria were frozen in aliquots at −80° C. Expression was verified by Western blotting (FIG. 2).

### Western Blotting

Listeria strains were grown in Luria-Bertoni medium at 37° C. and were harvested at the same optical density measured at 600 nm. The supernatants were TCA precipitated and resuspended in 1× sample buffer supplemented with 0.1 N NaOH. Identical amounts of each cell pellet or each TCA-precipitated supernatant were loaded on 4-20% Tris-glycine SDS-PAGE gels (NOVEX, San Diego, Calif.). The gels were transferred to polyvinylidene difluoride and probed with an anti-E7 monoclonal antibody (mAb) (Zymed Laboratories, South San Francisco, Calif.), then incubated with HRP-conjugated anti-mouse secondary Ab (Amersham Pharmacia Biotech, Little Chalfont, U.K.), developed with Amersham ECL detection reagents, and exposed to Hyperfilm (Amersham Pharmacia Biotech).

### Measurement of Tumor Growth

Tumors were measured every other day with calipers spanning the shortest and longest surface diameters. The mean of these two measurements was plotted as the mean tumor diameter in millimeters against various time points. Mice were sacrificed when the tumor diameter reached 20 mm. Tumor measurements for each time point are shown only for surviving mice.

### Effects of Listeria Recombinants on Established Tumor Growth

Six- to 8-wk-old C57BL/6 mice (Charles River) received 2×10⁵ TC-1 cells s.c. on the left flank. One week following tumor inoculation, the tumors had reached a palpable size of 4-5 mm in diameter. Groups of eight mice were then treated with 0.1 LD₅₀ i.p. Lm-LLO-E7 (10⁷ CFU), Lm-E7 (10⁶ CFU), Lm-LLO-NP (10⁷ CFU), or Lm-Gag (5×10⁵ CFU) on days 7 and 14.

### ⁵¹Cr Release Assay

C57BL/6 mice, 6-8 wk old, were immunized i.p. with 0.1 LD₅₀ Lm-LLO-E7, Lm-E7, Lm-LLO-NP, or Lm-Gag. Ten days post-immunization, spleens were harvested. Splenocytes were established in culture with irradiated TC-1 cells (100:1, splenocytes:TC-1) as feeder cells; stimulated in vitro for 5 days, then used in a standard ⁵¹Cr release assay, using the following targets: EL-4, EL-4/E7, or EL-4 pulsed with E7 H-2b peptide (RAHYNIVTF). E:T cell ratios, performed in triplicate, were 80:1, 40:1, 20:1, 10:1, 5:1, and 2.5:1. Following a 4-h incubation at 37° C., cells were pelleted, and 50 μl supernatant was removed from each well. Samples were assayed with a Wallac 1450 scintillation counter (Gaithersburg, Md.). The percent specific lysis was determined as [(experimental counts per minute (cpm)−spontaneous cpm)/(total cpm−spontaneous cpm)]×100.

TC-1-Specific Proliferation

C57BL/6 mice were immunized with 0.1 LD₅₀ and boosted by i.p. injection 20 days later with 1 LD₅₀ Lm-LLO-E7, Lm-E7, Lm-LLO-NP, or Lm-Gag. Six days after boosting, spleens were harvested from immunized and naive mice. Splenocytes were established in culture at 5×10⁵/well in flat-bottom 96-well plates with 2.5×10⁴, 1.25×10⁴, 6×10³, or 3×10³ irradiated TC-1 cells/well as a source of E7 Ag, or without TC-1 cells or with 10 μg/ml Con A. Cells were pulsed 45 h later with 0.5 μCi [³H]thymidine/well. Plates were harvested 18 h later using a Tomtec harvester 96 (Orange, Conn.), and proliferation was assessed with a Wallac 1450 scintillation counter. The change in cpm was calculated as experimental cpm−no Ag cpm.

Flow Cytometric Analysis

C57BL/6 mice were immunized intravenously (i.v.) with 0.1 LD₅₀ Lm-LLO-E7 or Lm-E7 and boosted 30 days later. Three-color flow cytometry for CD8 (53-6.7, PE conjugated), CD62 ligand (CD62L; MEL-14, APC conjugated), and E7 H-2Db tetramer was performed using a FACSCalibur® flow cytometer with CellQuest® software (Becton Dickinson, Mountain View, Calif.). Splenocytes harvested 5 days after the boost were stained at room temperature (rt) with H-2Db tetramers loaded with the E7 peptide (RAHYNIVTF) or a control (HIV-Gag) peptide. Tetramers were used at a 1/200 dilution and were provided by Dr. Larry R. Pease (Mayo Clinic, Rochester, Minn.) and by the NIAID Tetramer Core Facility and the NIH AIDS Research and Reference Reagent Program. Tetramer⁺, CD8⁺, CD62L^{low} cells were analyzed.

### B16F0-Ova Experiment

24 C57BL/6 mice were inoculated with 5×10⁵ B16F0-Ova cells. On days 3, 10 and 17, groups of 8 mice were immunized with 0.1 LD₅₀ Lm-OVA (10⁶ cfu), Lm-LLO-OVA (10⁸ cfu) and eight animals were left untreated.

### Statistics

For comparisons of tumor diameters, mean and SD of tumor size for each group were determined, and statistical significance was determined by Student's t test. p≤0.05 was considered significant.

### Results

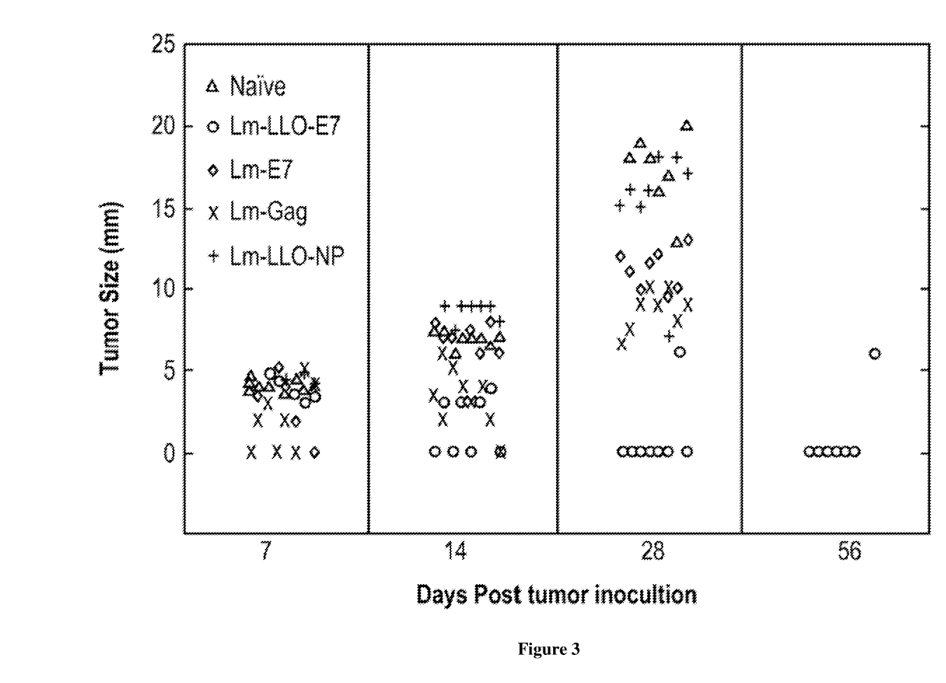

Lm-E7 and Lm-LLO-E7 were compared for their abilities to impact on TC-1 growth. Subcutaneous tumors were established on the left flank of C57BL/6 mice. Seven days later tumors had reached a palpable size (4-5 mm). Mice were vaccinated on days 7 and 14 with 0.1 LD₅₀ Lm-E7, Lm-LLO-E7, or, as controls, Lm-Gag and Lm-LLO-NP. Lm-LLO-E7 induced complete regression of 75% of established TC-1 tumors, while tumor growth was controlled in the other 2 mice in the group (FIG. 3). By contrast, immunization with Lm-E7 and Lm-Gag did not induce tumor regression. This experiment was repeated multiple times, always with very similar results. In addition, similar results were achieved for Lm-LLO-E7 under different immunization protocols. In another experiment, a single immunization was able to cure mice of established 5 mm TC-1 tumors.

In other experiments, similar results were obtained with 2 other E7-expressing tumor cell lines: C3 and EL-4/E7. To confirm the efficacy of vaccination with Lm-LLO-E7, animals that had eliminated their tumors were re-challenged with TC-1 or EL-4/E7 tumor cells on day 60 or day 40, respectively. Animals immunized with Lm-LLO-E7 remained tumor free until termination of the experiment (day 124 in the case of TC-1 and day 54 for EL-4/E7).

Thus, expression of an antigen as a fusion protein with ΔLLO enhances the immunogenicity of the antigen.

### EXAMPLE 2

### Lm-LLO-E7 Treatment Elicits TC-1 Specific Splenocyte Proliferation

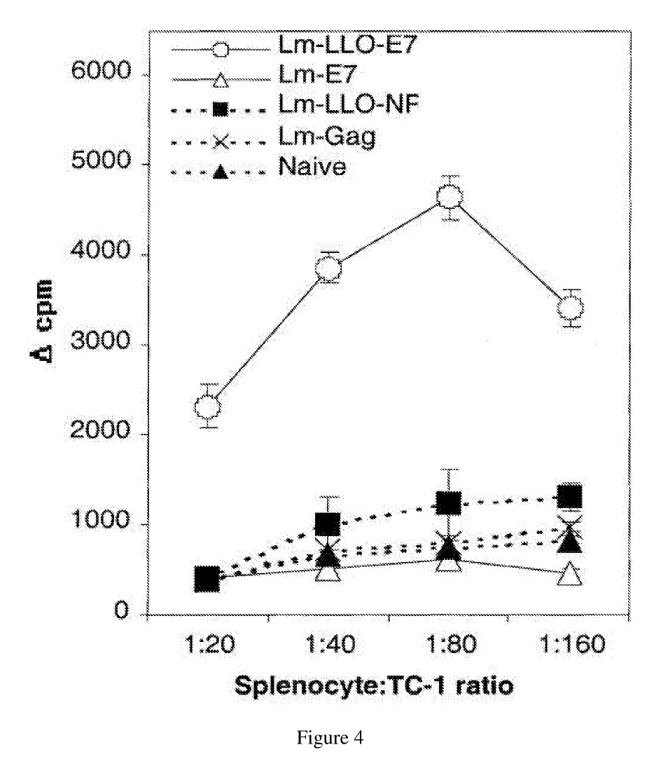

To measure induction of T cells by Lm-E7 with Lm-LLO-E7, TC-1-specific proliferative responses, a measure of antigen-specific immunocompetence, were measured in immunized mice. Splenocytes from Lm-LLO-E7-immunized mice proliferated when exposed to irradiated TC-1 cells as a source of E7, at splenocyte: TC-1 ratios of 20:1, 40:1, 80:1, and 160:1 (FIG. 4). Conversely, splenocytes from Lm-E7 and rLm control-immunized mice exhibited only background levels of proliferation.

### EXAMPLE 3

### ActA-E7 and PEST-E7 Fusions Confer Anti-Tumor Immunity

### Materials and Experimental Methods

### Construction of Lm-ActA-E7

Lm-ActA-E7 is a recombinant strain of LM, comprising a plasmid that expresses the E7 protein fused to a truncated version of the actA protein. Lm-actA-E7 was generated by introducing a plasmid vector pDD-1, constructed by modifying pDP-2028, into Listeria. pDD-1 comprises an expression cassette expressing a copy of the 310 bp hly promoter and the hly signal sequence (ss), which drives the expression and secretion of ActA-E7; 1170 bp of the actA gene that comprises four PEST sequences (SEQ ID NO: 5) (the truncated ActA polypeptide consists of the first 390 AA of the molecule, SEQ ID NO: 4); the 300 bp HPV E7 gene; the 1019 bp prfA gene (controls expression of the virulence genes); and the CAT gene (chloramphenicol resistance gene) for selection of transformed bacteria clones (Sewell et al. (2004), Arch. Otolaryngol. Head Neck Surg., 130: 92-97).

The hly promoter (pHly) and gene fragment were PCR amplified from pGG55 (Example 1) using primer 5′-GGGGTCTAGACCTCCTTTGATTAGTATATTC-3′ (Xba I site is underlined; SEQ ID NO: 25) and primer 5′-ATCTTCGCTATCTGTCGCCGCGGCGCGTGCTTCAGTTTGTTGCGC-'3 (Not I site is underlined. The first 18 nucleotides are the ActA gene overlap; SEQ ID NO: 26). The actA gene was PCR amplified from the LM 10403s wildtype genome using primer 5′-GCGCAACAAACTGAAGCAGCGGCCGCGGCGACAGATAGCGAAGAT-3′ (NotI site is underlined; SEQ ID NO: 27) and primer 5′-TGTAGGTGTATCTCCATGCTCGAGAGCTAGGCGATCAATTTC-3′ (XhoI site is underlined; SEQ ID NO: 28). The E7 gene was PCR amplified from pGG55 (pLLO-E7) using primer 5′-GGAATTGATCGCCTAGCTCTCGAGCATGGAGATACACCTACA-3′ (XhoI site is underlined; SEQ ID NO: 29) and primer 5′-AAACGGATTTATTTAGATCCCGGGTTATGGTTTCTGAGAACA-3′ (XmaI site is underlined; SEQ ID NO: 30). The prfA gene was PCR amplified from the LM 10403s wild-type genome using primer 5′-TGTTCTCAGAAACCATAACCCGGGATCTAAATAAATCCGTTT-3′ (XmaI site is underlined; SEQ ID NO: 31) and primer 5′-GGGGGTCGACCAGCTCTTCTTGGTGAAG-3′ (SalI site is underlined; SEQ ID NO: 32). The hly promoter-actA gene fusion (pHly-actA) was PCR generated and amplified from purified pHly DNA and purified actA DNA using the upstream pHly primer (SEQ ID NO: 25) and downstream actA primer (SEQ ID NO: 28).

The E7 gene fused to the prfA gene (E7-prfA) was PCR generated and amplified from purified E7 DNA and purified prfA DNA using the upstream E7 primer (SEQ ID NO: 29) and downstream prfA gene primer (SEQ ID NO: 32).

The pHly-actA fusion product fused to the E7-prfA fusion product was PCR generated and amplified from purified fused pHly-actA DNA product and purified fused E7-prfA DNA product using the upstream pHly primer (SEQ ID NO: 25) and downstream prfA gene primer (SEQ ID NO: 32) and ligated into pCRII (Invitrogen, La Jolla, Calif.). Competent E. coli (TOP10′F, Invitrogen, La Jolla, Calif.) were transformed with pCRII-ActAE7. After lysis and isolation, the plasmid was screened by restriction analysis using BamHI (expected fragment sizes 770 bp and 6400 bp (or when the insert was reversed into the vector: 2500 bp and 4100 bp)) and BstXI (expected fragment sizes 2800 bp and 3900 bp) and also screened with PCR analysis using the upstream pHly primer (SEQ ID NO: 25) and the downstream prfA gene primer (SEQ ID NO: 32).

The pHly-actA-E7-prfA DNA insert was excised from pCRII by double digestion with Xba I and Sal I and ligated into pDP-2028 also digested with Xba I and Sal I. After transforming TOP10′F competent E. coli (Invitrogen, La Jolla, Calif.) with expression system pActAE7, chloramphenicol resistant clones were screened by PCR analysis using the upstream pHly primer (SEQ ID NO: 25) and the downstream PrfA gene primer (SEQ ID NO: 32). A clone comprising pActAE7 was grown in brain heart infusion medium (with chloramphenicol (20 mcg (microgram)/ml (milliliter), Difco, Detroit, Mich.) and pActAE7 was isolated from the bacteria cell using a midiprep DNA purification system kit (Promega, Madison, Wis.). A prfA-negative strain of penicillin-treated Listeria (strain XFL-7) was transformed with expression system pActAE7, as described in Ikonomidis et al. (1994, J. Exp. Med. 180: 2209-2218) and clones were selected for the retention of the plasmid in vivo. Clones were grown in brain heart infusion with chloramphenicol (20 mcg/ml) at 37° C. Bacteria were frozen in aliquots at −80° C.

### Immunoblot Verification of Antigen Expression

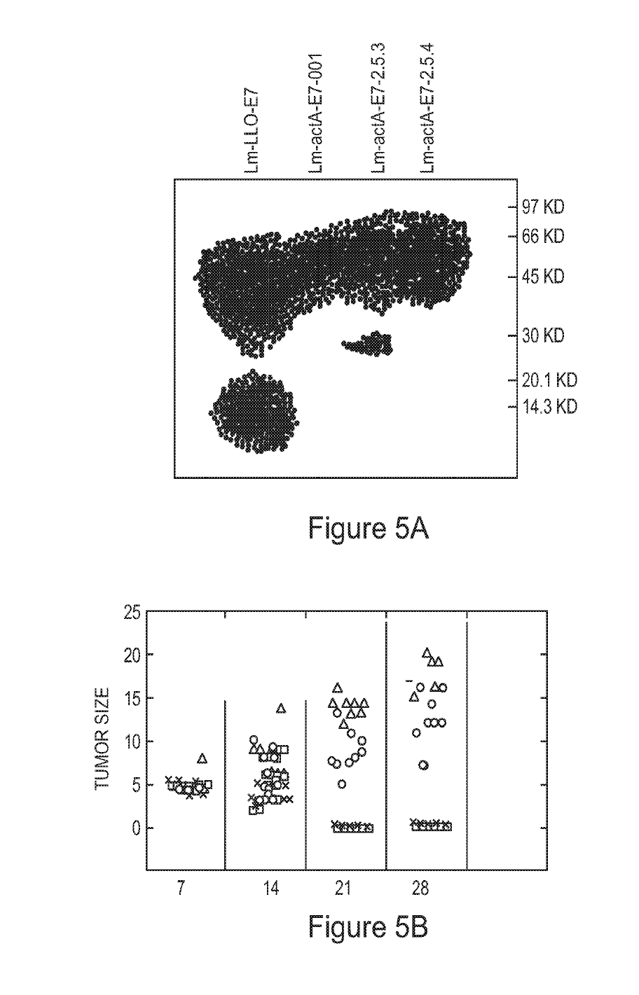

To verify that Lm-ActA-E7 secretes ActA-E7, (about 64 kD), Listeria strains were grown in Luria-Bertoni (LB) medium at 37° C. Protein was precipitated from the culture supernatant with trichloroacetic acid (TCA) and resuspended in 1× sample buffer with 0.1N sodium hydroxide. Identical amounts of each TCA precipitated supernatant were loaded on 4% to 20% Tris-glycine sodium dodecyl sulfate-polyacrylamide gels (NOVEX, San Diego, Calif). Gels were transferred to polyvinylidene difluoride membranes and probed with 1:2500 anti-E7 monoclonal antibody (Zymed Laboratories, South San Francisco, Calif), then with 1:5000 horseradish peroxidase-conjugated anti-mouse IgG (Amersham Pharmacia Biotech, Little Chalfont, England). Blots were developed with Amersham enhanced chemiluminescence detection reagents and exposed to autoradiography film (Amersham) (FIG. 5A).

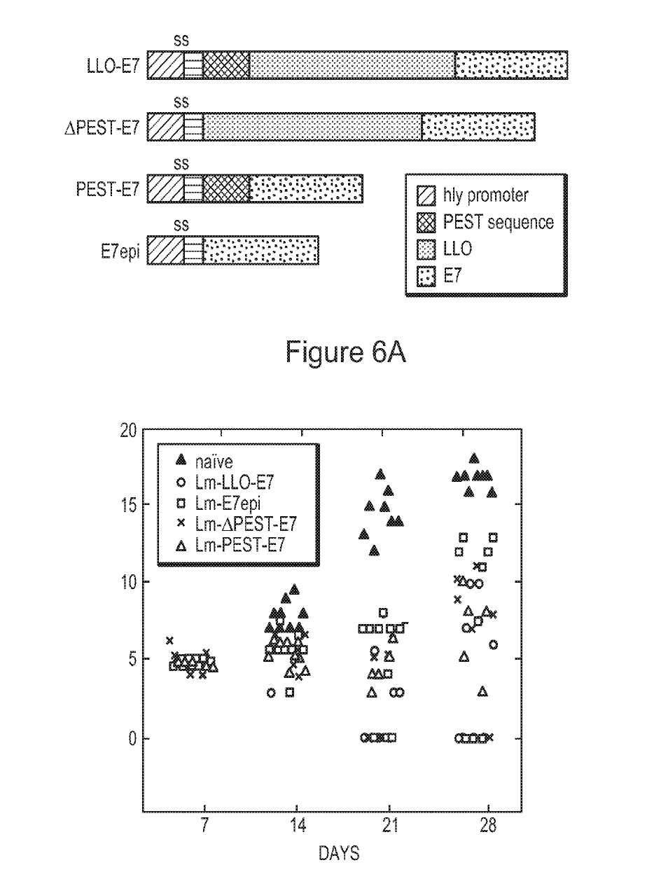

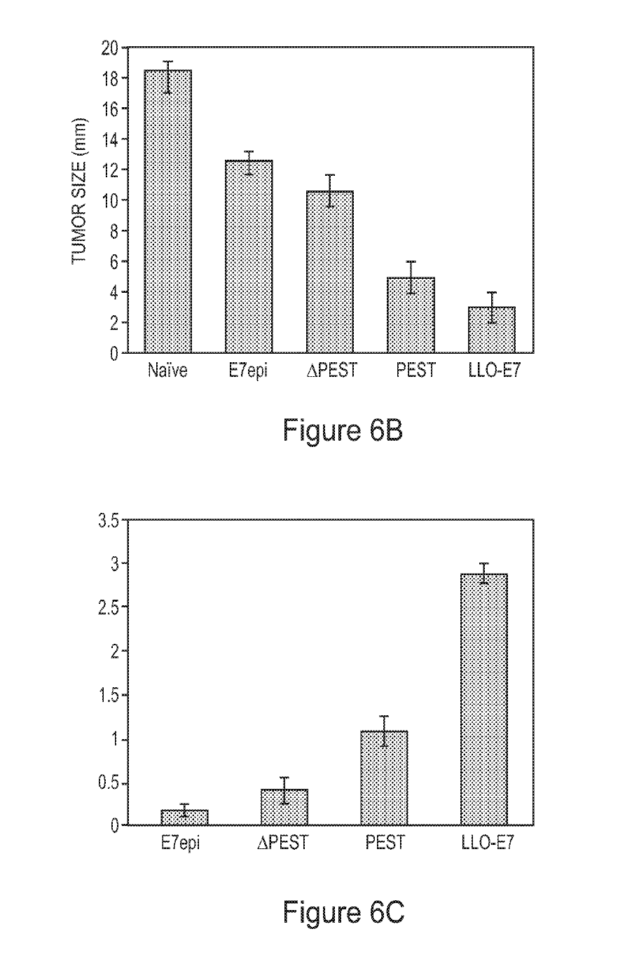

### Construction of Lm-PEST-E7, Lm-ΔPEST-E7, and Lm-E7epi (FIG. 6A)

Lm-PEST-E7 is identical to Lm-LLO-E7, except that it contains only the promoter and PEST sequence of the hly gene, specifically the first 50 AA of LLO. To construct Lm-PEST-E7, the hly promoter and PEST regions were fused to the full-length E7 gene using the SOE (gene splicing by overlap extension) PCR technique. The E7 gene and the hly-PEST gene fragment were amplified from the plasmid pGG-55, which contains the first 441 AA of LLO, and spliced together by conventional PCR techniques. To create a final plasmid, pVS16.5, the hly-PEST-E7 fragment and the prfA gene were subcloned into the plasmid pAM401, which includes a chloramphenicol resistance gene for selection in vitro, and the resultant plasmid was used to transform XFL-7.

Lm-ΔPEST-E7 is a recombinant Listeria strain that is identical to Lm-LLO-E7 except that it lacks the PEST sequence. It was made essentially as described for Lm-PEST-E7, except that the episomal expression system was constructed using primers designed to remove the PEST-containing region (bp 333-387) from the hly-E7 fusion gene. Lm-E7epi is a recombinant strain that secretes E7 without the PEST region or LLO. The plasmid used to transform this strain contains a gene fragment of the hly promoter and signal sequence fused to the E7 gene. This construct differs from the original Lm-E7, which expressed a single copy of the E7 gene integrated into the chromosome. Lm-E7epi is completely isogenic to Lm-LLO-E7, Lm-PEST-E7, and Lm-ΔPEST-E7 except for the form of the E7 antigen expressed.

### Results

To compare the anti-tumor immunity induced by Lm-ActA-E7 versus Lm-LLO-E7, 2×10⁵ TC-1 tumor cells were implanted subcutaneously in mice and allowed to grow to a palpable size (approximately 5 millimeters [mm]). Mice were immunized i.p. with one LD₅₀ of either Lm-ActA-E7 (5×10⁸ CFU), (crosses) Lm-LLO-E7 (10⁸ CFU) (squares) or Lm-E7 (10⁶ CFU) (circles) on days 7 and 14. By day 26, all of the animals in the Lm-LLO-E7 and Lm-ActA-E7 were tumor free and remained so, whereas all of the naive animals (triangles) and the animals immunized with Lm-E7 grew large tumors (FIG. 5B). Thus, vaccination with ActA-E7 fusions causes tumor regression.

In addition, Lm-LLO-E7, Lm-PEST-E7, Lm-ΔPEST-E7, and Lm-E7epi were compared for their ability to cause regression of E7-expressing tumors. S.c. TC-1 tumors were established on the left flank of 40 C57BL/6 mice. After tumors had reached 4-5 mm, mice were divided into 5 groups of 8 mice. Each groups was treated with 1 of 4 recombinant LM vaccines, and 1 group was left untreated. Lm-LLO-E7 and Lm-PEST-E7 induced regression of established tumors in ⅝ and ⅜ cases, respectively. There was no statistical difference between the average tumor size of mice treated with Lm-PEST-E7 or Lm-LLO-E7 at any time point. However, the vaccines that expressed E7 without the PEST sequences, Lm-ΔPEST-E7 and Lm-E7epi, failed to cause tumor regression in all mice except one (FIG. 6B, top panel). This was representative of 2 experiments, wherein a statistically significant difference in mean tumor sizes at day 28 was observed between tumors treated with Lm-LLO-E7 or Lm-PEST-E7 and those treated with Lm-E7epi or Lm-ΔPEST-E7; P<0.001, Student's t test; FIG. 6B, bottom panel). In addition, increased percentages of tetramer-positive splenocytes were seen reproducibly over 3 experiments in the spleens of mice vaccinated with PEST-containing vaccines (FIG. 6C). Thus, vaccination with PEST-E7 fusions causes tumor regression.

### EXAMPLE 4

### Fusion of E7 to LLO, ActA, or a Pest-Like Sequence Enhances E7-Specific Immunity and Generates Tumor-Infiltrating E7-Specific CD8⁺ Cells

### Materials and Experimental Methods

500 mcl (microliter) of MATRIGEL®, comprising 100 mcl of 2×10⁵ TC-1 tumor cells in phosphate buffered saline (PBS) plus 400 mcl of MATRIGEL® (BD Biosciences, Franklin Lakes, N.J.) were implanted subcutaneously on the left flank of 12 C57BL/6 mice (n=3). Mice were immunized intraperitoneally on day 7, 14 and 21, and spleens and tumors were harvested on day 28. Tumor MATRIGELs were removed from the mice and incubated at 4° C. overnight in tubes containing 2 milliliters (ml) of RP 10 medium on ice. Tumors were minced with forceps, cut into 2 mm blocks, and incubated at 37° C. for 1 hour with 3 ml of enzyme mixture (0.2 mg/ml collagenase-P, 1 mg/ml DNAse-1 in PBS). The tissue suspension was filtered through nylon mesh and washed with 5% fetal bovine serum +0.05% of NaN₃ in PBS for tetramer and IFN-gamma staining.

Splenocytes and tumor cells were incubated with 1 micromole (mcm) E7 peptide for 5 hours in the presence of brefeldin A at 10⁷ cells/ml. Cells were washed twice and incubated in 50 mcl of anti-mouse Fc receptor supernatant (2.4 G2) for 1 hour or overnight at 4° C. Cells were stained for surface molecules CD8 and CD62L, permeabilized, fixed using the permeabilization kit Golgi-stop® or Golgi-Plug® (Pharmingen, San Diego, Calif.), and stained for IFN-gamma. 500,000 events were acquired using two-laser flow cytometer FACSCalibur and analyzed using Cellquest Software (Becton Dickinson, Franklin Lakes, N.J.). Percentages of IFN-gamma secreting cells within the activated (CD62L^{low}) CD8⁺ T cells were calculated.

For tetramer staining, H-2D^{b} tetramer was loaded with phycoerythrin (PE)-conjugated E7 peptide (RAHYNIVTF, SEQ ID NO: 33), stained at rt for 1 hour, and stained with anti-allophycocyanin (APC) conjugated MEL-14 (CD62L) and FITC-conjugated CD8␣ at 4° C. for 30 min. Cells were analyzed comparing tetramer⁺CD8⁺ CD62L^{low} cells in the spleen and in the tumor.

### RESULTS

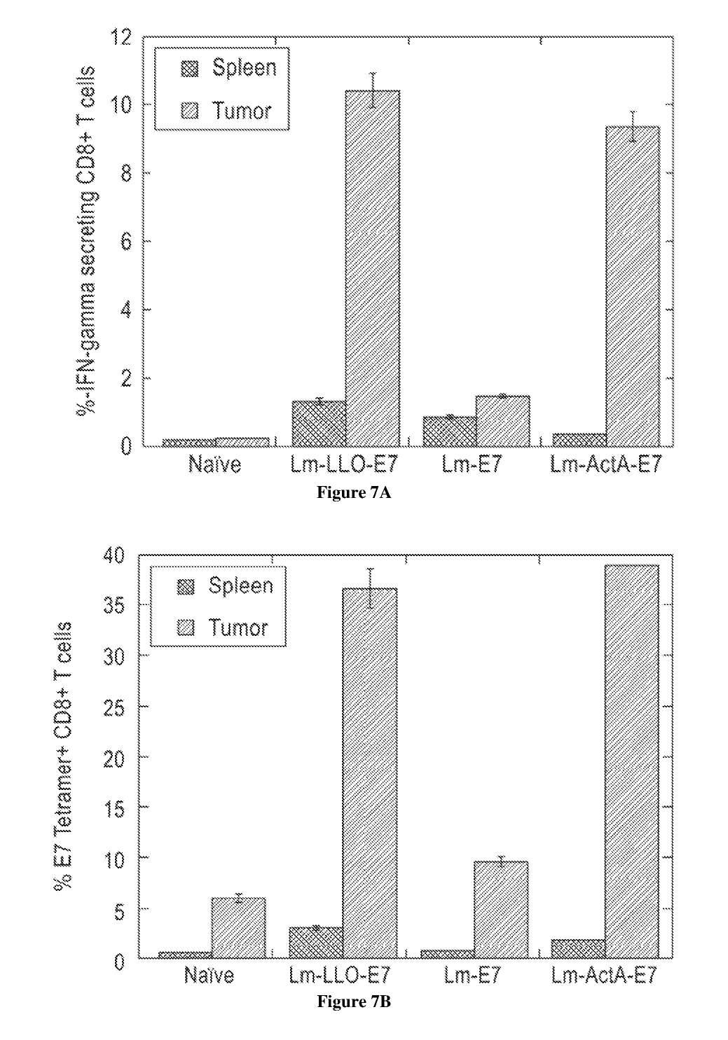

To analyze the ability of Lm-ActA-E7 to enhance antigen specific immunity, mice were implanted with TC-1 tumor cells and immunized with either Lm-LLO-E7 (1×10⁷ CFU), Lm-E7 (1×10⁶ CFU), or Lm-ActA-E7 (2×10⁸ CFU), or were untreated (naïve). Tumors of mice from the Lm-LLO-E7 and Lm-ActA-E7 groups contained a higher percentage of IFN-gamma-secreting CD8⁺ T cells (FIG. 7A) and tetramer-specific CD8⁺ cells (FIG. 7B) than in Lm-E7 or naive mice.

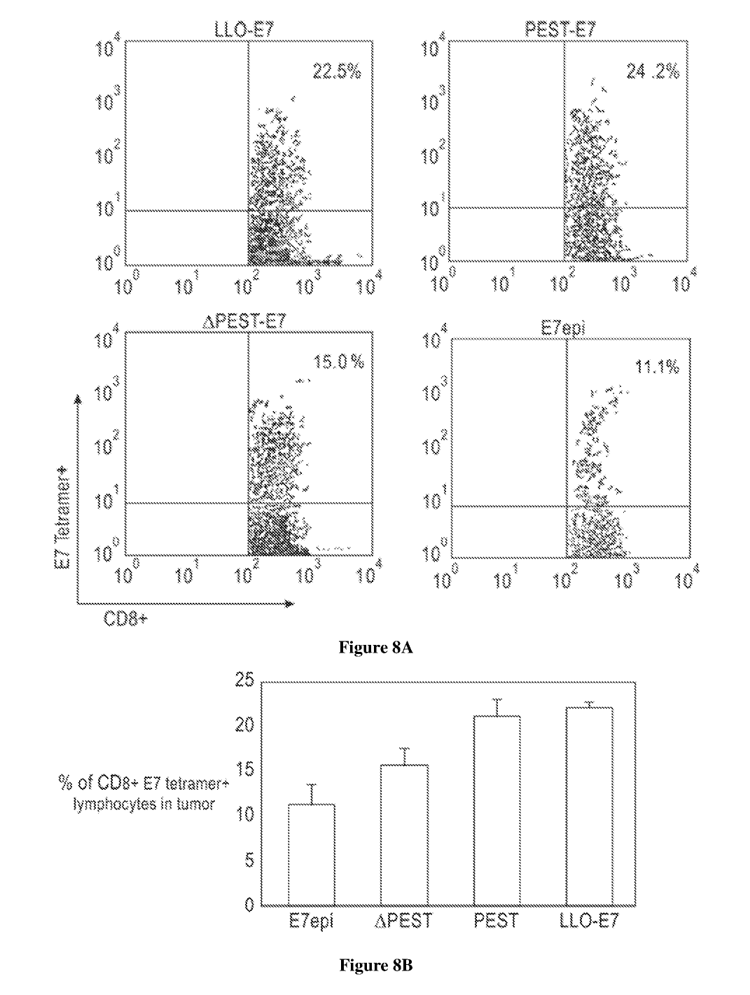

In another experiment, tumor-bearing mice were administered Lm-LLO-E7, Lm-PEST-E7, Lm-ΔPEST-E7, or Lm-E7epi, and levels of E7-specific lymphocytes within the tumor were measured. Mice were treated on days 7 and 14 with 0.1 LD₅₀ of the 4 vaccines. Tumors were harvested on day 21 and stained with antibodies to CD62L, CD8, and with the E7/Db tetramer. An increased percentage of tetramer-positive lymphocytes within the tumor were seen in mice vaccinated with Lm-LLO-E7 and Lm-PEST-E7 (FIG. 8A). This result was reproducible over three experiments (FIG. 8B).

Thus, Lm-LLO-E7, Lm-ActA-E7, and Lm-PEST-E7 are each efficacious at induction of tumor-infiltrating CD8⁺ T cells and tumor regression.

### EXAMPLE 5

### E6/E7 Transgenic Mouse Phenotype: a Model for Spontaneous Tumor Growth and Tolerance to a Tumor Antigen

### Materials and Experimental Methods

Several C57BL/6 mouse zygotes were injected with plasmids containing the HPV-16 E6/E7 gene under the control of the thyroglobulin promoter (provided by M Parmentier, Brussels). Tail clippings of several litters were screened via PCR for the E6/E7 gene. The E7 gene and the thyroglobulin promoter were integrated into the majority of the progeny. Positive mosaic E7 transgenic mice were then selected for F0×wild type breeding. Subsequent F1 generations were screened, via PCR, for the presence of the E7 gene. E7 positive pups generated from F0×wt breeding pairs were selected for F1×F1 breeding. The zygosity of F1 breeding pair derived generations was determined by Taqman real-time PCR and the ΔCt method (Charles River, 2001). Homozygous E7 transgenic mice were selected for F2×F2 breeding. The subsequent F3 generation was screened via Taqman real-time PCR and backcrossing to confirm fidelity of homozygosity. The levels of gene copy number and transgene expression of the E7 gene was assessed for every homozygous line using Taqman real-time PCR. After 6 back-crossings, these lines were used as the parents of the colony. Transgene expression was further confirmed by appearance of thyroid hyperplasia, as described in the Results section.

### Results

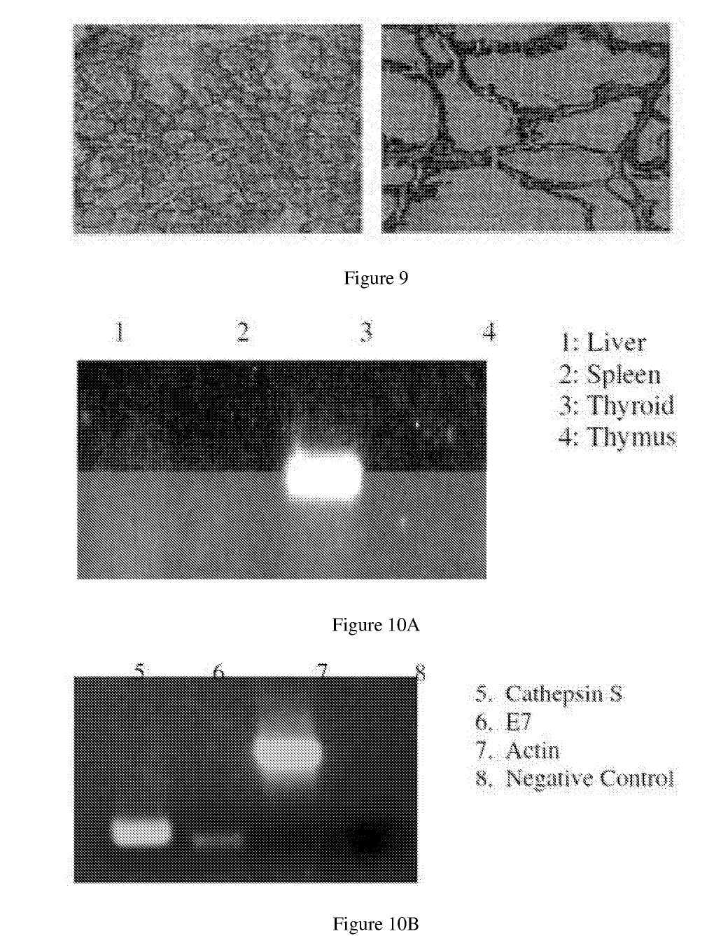

E6/E7 transgenic mice were generated, and their phenotype assessed. The mice began to develop thyroid hyperplasia at 8 weeks and palpable goiters at 6 months. By 6 to 8 months, most mice exhibited thyroid cancer. Transgenic mice sacrificed at 3 months of age exhibited de-differentiation of the normal thyroid architecture, indicative of an early stage of cancer. The enlarged, de-differentiated cells were filled with colloid, where thyroid hormones accumulate (FIG. 9).

### EXAMPLE 6

### E7 is Expressed in Medullary Thymic Epithelial Cells of E6/E7 Transgenic Mice

To determine whether or not E7 was expressed in the thymus, liver, spleen, thymus and thyroid were examined for the expression of the transgene in 6 to 8 week old mice. Abundant E7 message was found in the thyroid but not in other tissues (FIG. 10A). The absence of E7 message in whole thymus preparations was not indicative of lack of expression in the thymus, since the level of message of a peripherally expressed, organ-specific antigen, including thyroglobulin, has been shown to be too low to detect in whole thymocyte preparations (Derbinski, J., A. Schulte, B. Kyewski, and L. Klein. 2001. Promiscuous gene expression in medullary thymic epithelial cells mirrors the peripheral self. Nat Immunol 2:1032).

Tolerance to peripheral antigens in the thymus, including thyroglobulin, is mediated by the transient expression of these genes by the autoimmune regulator (AIRE) in thymic medullary epithelial cells (mTECs), with peak expression occurring prior to birth. AIRE is a transcription factor that maintains tolerance to self. To determine whether E7 expression in the transgenic mice followed the same pattern, mTECs from E6/E7 thymi of young mice (3-5 weeks) were examined for E7 expression.

The mTECs expressed E7 message, and also expressed Cathepsin S, which is known to be expressed in mTECs (FIG. 10B). Thus, E7 is expressed in the thymus of the transgenic mice, showing that these mice exhibit tolerance to the E7 antigen.

### EXAMPLE 7

### Peptide-Based Vaccines Do Not Protect Against Tumor Challenge in E6/E7 Transgenic Mice

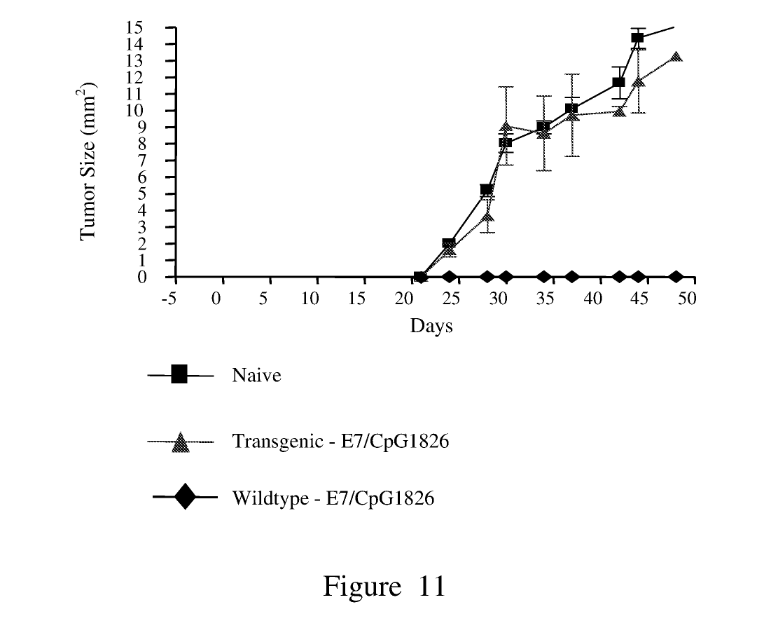

As a measure of the impact of the self-expression of E7 on vaccine efficacy, E6/E7 transgenic mice were tested in a tumor protection experiment using an E7 peptide (RAHYNIVTF)-based vaccine, along with the immunostimulatory CpG sequence 1826 (Krieg A M, Yi A K, Matson S, Waldschmidt T J, Bishop G A, Teasdale R, Koretzky G A, Klinman D M. Nature 374:546). While the peptide-based vaccine protected all the wild type mice from tumor challenge, it had no impact on tumor challenge in the transgenic mouse (FIG. 11). Thus, the E6/E7 mice exhibit reduced ability to reject tumor challenge, providing further evidence that they are tolerant to E7.

### EXAMPLE 8

### LLO and ActA Fusions Overcome Immune Tolerance of E6/E7 Transgenic Mice to E7-Expressing Tumors

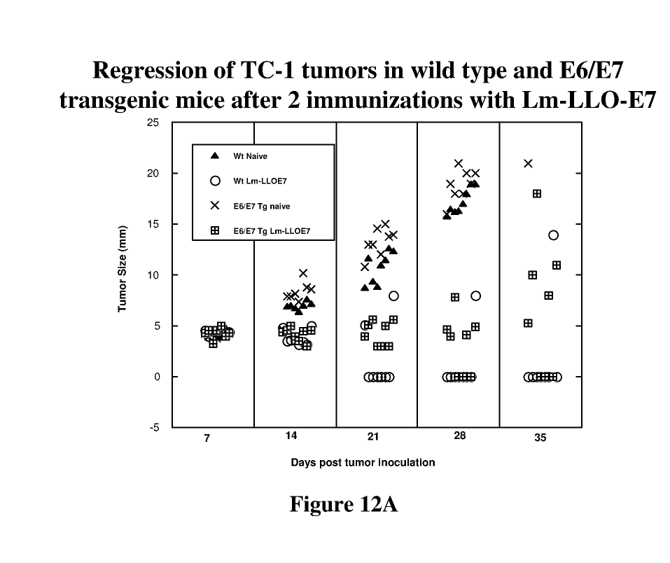

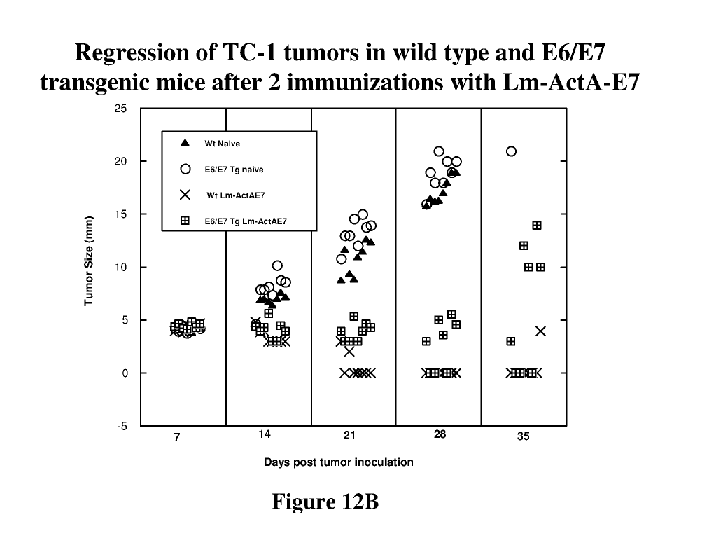

To test the ability of vaccines of the present invention to overcome the immune tolerance of E6/E7 transgenic mice to E7-expressing tumors, 10⁵ TC-1 cells were implanted subcutaneously (s.c.) and allowed to form solid tumors in 6-8 week old wild-type and transgenic mice 7 and 14 days later, mice were left unimmunized or were immunized i.p. with LM-NP (control), 1×10⁸ cfu LM-LLO-E7 (FIG. 12A) or 2.5×10⁸ cfu LM-ActA-E7 (FIG. 12B). The naïve mice had a large tumor burden, as anticipated, and were sacrificed by day 28 or 35 due to tumors of over 2 cm. By contrast, by day 35, administration of either LM-LLO-E7 or LM-ActA-E7 resulted in complete tumor regression in ⅞ or 6/8, respectively, of the wild-type mice and ⅜ of the transgenic mice. In the transgenic mice that did not exhibit complete tumor regression, a marked slowing of tumor growth was observed in the LM-LLO-E7-vaccinated and LM-ActA-E7-vaccinated mice.

The effectiveness of vaccines of the present invention in inducing complete tumor regression and/or slowing of tumor growth in transgenic mice was in marked contrast to the inefficacy of the peptide-based vaccine. Thus, vaccines of the present invention were able to overcome immune tolerance of E6/E7 transgenic mice to E7-expressing tumors.

### EXAMPLE 9

### LLO AND ActA Fusions Reduce Autochthonous (Spontaneous) Tumors in E6/E7 Transgenic Mice

To determine the impact of the Lm-LLO-E7 and Lm-ActA-E7 vaccines on autochthonous tumors in the E6/E7 transgenic mouse, 6 to 8 week old mice were immunized with 1×10⁸ Lm-LLO-E7 or 2.5×10⁸ Lm-ActA-E7 once per month for 8 months. Mice were sacrificed 20 days after the last immunization and their thyroids removed and weighed. This experiment was performed twice (Table 1).

TABLE 1
Thyroid weight (mg) in unvaccinated and vaccinated
transgenic mice at 8 months of age (mg)*.

| | | | | | | Lm- | |
|---|---|---|---|---|---|---|---|
| | | Lm-LLO- | | Lm-LLO- | | ActA- | |
| Untreated | ±S.D. | NP | ±S.D. | E7 | ±S.D. | E7 | ±S.D. |
| Expt. 1 | 123 | 385 | 130 | 225 | 54 | 305 | 92 |
| 408 | | | | | | | |
| Expt. 2 | 94 | 503 | 86 | 239 | 68 | 275 | 84 |
| 588 | | | | | | | |

*Statistical analyses performed using Student's t test showed that the difference in thyroid weight between Lm-LLO-NP treated mice and untreated mice was not significant but that the difference between Lm-LLO-E7 and Lm-ActA-E7 treated mice was highly significant (p < 0.001)

The difference in thyroid weight between Lm-LLO-E7 treated mice and untreated mice and between Lm-LLO-ActA treated mice and untreated mice was significant (p<0.001 and p<0.05, respectively) for both experiments, while the difference between Lm-LLO-NP treated mice (irrelevant antigen control) and untreated mice was not significant (Student's t test), showing that Lm-LLO-E7 and Lm-ActA-E7 controlled spontaneous tumor growth. Thus, vaccines of the present invention prevent formation of new E7-expressing tumors.

To summarize the findings in the above Examples, LLO-antigen and ActA-antigen fusions (a) induce tumor-specific immune response that include tumor-infiltrating antigen-specific T cells; and are capable of inducing tumor regression and controlling tumor growth of both normal and particularly aggressive tumors; (b) overcome tolerance to self antigens; and (c) prevent spontaneous tumor growth. These findings are generalizable to a large number of antigens, PEST-like sequences, and tumor types, as evidenced by their successful implementation with a variety of different antigens, PEST-like sequences, and tumor types.

### EXAMPLE 10

### LM-LLO-E7 Vaccines Are Safe and Improve Clinical Indicators in Cervical Cancer Patients

### Materials and Experimental Methods

Inclusion criteria. All patients in the trial were diagnosed with “advanced, progressive or recurrent cervical cancer,” and an assessment at the time of entry indicated that all were staged as having IVB disease. All patients manifested a positive immune response to an anergy panel containing 3 memory antigens selected from candidin, mumps, tetanus, or Tuberculin Purified Protein Derivative (PPD); were not pregnant or HIV positive, had taken no investigational drugs within 4 weeks, and were not receiving steroids.

Protocol: Patients were administered 2 vaccinations at a 3-week interval as a 30-minute intravenous (IV) infusion in 250 ml of normal saline to inpatients. After 5 days, patients received a single course of IV ampicillin and were released with an additional 10 days of oral ampicillin. Karnofsky Performance Index, which is a measurement of overall vitality and quality of life such as appetite, ability to complete daily tasks, restful sleep, etc, was used to determine overall well-being. In addition, the following indicators of safety and general well being were determined: alkaline phosphatase; bilirubin, both direct and total; gamma glutamyl transpeptidase (ggt); cholesterol; systole, diastole, and heart rate; Eastern Collaborative Oncology Group's (ECOG)'s criteria for assessing disease progression—a Karnofsky like—quality of life indicator; hematocrit; hemoglobin; platelet levels; lymphocytes levels; AST (aspartate aminotransferase); ALT (alanine aminotransferase); and LDH (lactate dehydrogenase). Patients were followed at 3 weeks and 3 months subsequent to the second dosing, at which time Response Evaluation Criteria in Solid Tumors (RECIST) scores of the patients were determined, scans were performed to determine tumor size, and blood samples were collected for immunological analysis at the end of the trial, which includes the evaluation of IFN-γ, IL-4, CD4⁺ and CD8⁺ cell populations.

Listeria strains: The creation of LM-LLO-E7 is described in Example 1. Bacteria were passaged twice through mice prior to preparation of the working cell bank, as described in Example 12. The cell bank exhibited viability upon thawing of greater than 90%.

### Results

Prior to the clinical trial, a preclinical experiment was performed to determine the anti-tumor efficacy of intravenous (i.v.) vs. i.p. administration of LM-LLO-E7. A tumor containing 1×10⁴ TC-1 cells was established sub-cutaneously. On days 7 and 14, mice were immunized with either 10⁸ LM-LLO-E7 i.p. or LM-LLO-E7 i.v. at doses of 10⁸, 10⁷, 10⁶, or 10⁵. At day 35, ⅝ of the mice that received 10⁸ LM-LLO-E7 by either route or 10⁷ LM-LLO-E7 i.v, and 4/8 of the mice that received 10⁶ LM-LLO-E7 i.v, were cured. By contrast, doses of less than 10⁷ or in some cases even 10⁸ LM-LLO-E7 administered i.p. were ineffective at controlling tumor growth. Thus, i.v. administration of LM-LLO-E7 is more effective than i.p. administration.

### Clinical Trial

A phase I/II clinical trial was conducted to assess safety and efficacy of LM-LLO-E7 vaccines in patients with advanced, progressive, or recurrent cervical cancer. 5 patients each were assigned to cohorts 1-2, which received 1×10⁹ or 3.3×10⁹ CFU, respectfully. An additional 5 patients each will be assigned to cohorts 3-4, which will receive 1×10¹⁰ or 3.31×10¹⁰ CFU, respectfully.

### Safety Data

First cohort

All patients in the first cohort reported onset of mild-to-moderate fever and chills within 1-2 hours after onset of the infusion. Some patients exhibited vomiting, with or without nausea. With 1 exception (described below), a single dose of a non-steroidal agent such as paracetamol was sufficient to resolve these symptoms. Modest, transient cardiovascular effects were observed, consistent with, and sharing the time course of, the fever. No other adverse effects were reported.

At this late stage of cervical cancer, 1 year survival is typically 10-15% of patients and no tumor therapy has ever been effective. Indeed, Patient 2 was a young patient with very aggressive disease who passed away shortly after completing the trial.

Quantitative blood cultures were assessed on days 2, 3, and 5 post-administration. Of the 5 evaluable patients in this cohort, 4 exhibited no serum Listeria at any time and 1 had a very small amount (35 cfu) of circulating Listeria on day 2, with no detectable Listeria on day 3 or 5.

Patient 5 responded to initial vaccination with mild fever over the 48 hours subsequent to administration, and was treated with anti-inflammatory agents. On 1 occasion, the fever rose to moderate severity (at no time above 38.4° C.), after which she was given a course of ampicillin, which resolved the fever. During the antibiotic administration she experienced mild urticaria, which ended after antibiotic administration. Blood cultures were all sterile, cardiovascular data were within the range observed for other patients, and serum chemistry values were normal, showing that this patient had no listerial disease. Further, the anergy panel indicated a robust response to ⅓ memory antigens, indicating the presence of functional immunity (similar to the other patients). Patient 5 subsequently evidenced a response similar to all other patients upon receiving the boost.

### Second Cohort and Overall Safety Observations

In both cohorts, minor and transient changes in liver function tests were observed following infusion. These changes were determined by the attending physician monitoring the trial to have no clinical significance, and were expected for a short-lived infection of bacteria that are rapidly removed from the systemic circulation to the liver and spleen. In general, all the safety indicators described in the Methods section above displayed little or no net change, indicative of an excellent safety profile. The side effect profile in this cohort was virtually identical to that seen in the in the initial cohort and appeared to be a dose independent series of symptoms related to the consequences of cytokines and similar agents that occur consequent to the induction of an iatrogenic infection. No serum Listeria was observed at any time and no dose limiting toxicity was observed in either cohort.

### Efficacy—First Cohort

The following indications of efficacy were observed in the 3 patients in the first cohort that finished the trial: (Table 2).

Patient 1 entered the trial with 2 tumors of 20 mm each, which shrunk to 18 and 14 mm over the course of the trial, indicating therapeutic efficacy of the vaccine. In addition, patient 1 entered the trial with a Karnofsky Performance Index of 70, which rose to 90 after dosing. In the Safety Review Panel meeting, Siniša Radulovic, the chairman of the Department of Oncology, Institute for Oncology and Radiology, Belgrade, Serbia presented the results to a representative of the entity conducting the trials; Michael Kurman, an independent oncologist who works as a consultant for the entity; Kevin Ault, an academic gynecologic oncologist at Emory University who conducted the phase III Gardasil trials for Merck and the Cervarix trials for Glaxo SmithKline; and Tate Thigpen, a founder of the Gynecologic Oncology Group at NCI and professor of gynecologic oncology at the University of Mississippi. In the opinion of Dr. Radulovic, patient 1 exhibited a clinical benefit from treatment with the vaccine.

Before passing away, Patient 2 exhibited a mixed response, with ½ tumors shrinking.

Patient 3 enrolled with paraneoplastic disease, (an epiphenomenon of cancer wherein the overall debilitated state of the patient has other sequelae that are secondary to the cancer), including an elevation of platelet count to 936×10⁹/ml. The count decreased to 465×10⁹/ml, approximately a normal level, following the first dose.

Patient 4 entered the trial with 2 tumors of 20 mm each, which shrunk to 18 and 14 mm over the course of the trial, indicating therapeutic efficacy of the vaccine. Patient 4 exhibited a weight gain of 1.6 Kg and an increased hemoglobin count of approximately 10% between the first and second doses.

### Efficacy—Second Cohort and General Observations

In the lowest dose cohort, 2 patients demonstrated the shrinkage of tumors. The timing of this effect was consistent with that observed in immunological responses, in that it followed chronologically development of the immune response. One of the 2 patients in the second cohort evaluated so far for tumor burden exhibited a dramatic tumor load reduction at a post-vaccination time point. At the start of the trial, this patient had 3 tumors of 13, 13, and 14 mm. After the 2 doses of the vaccine, 2 of the tumor had shrunk to 9.4 and 12 mm, and the third was no longer detectable.

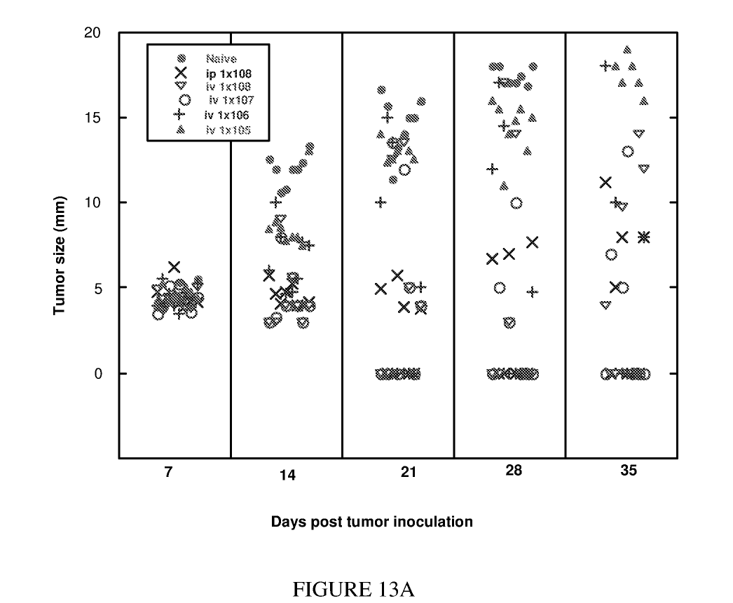

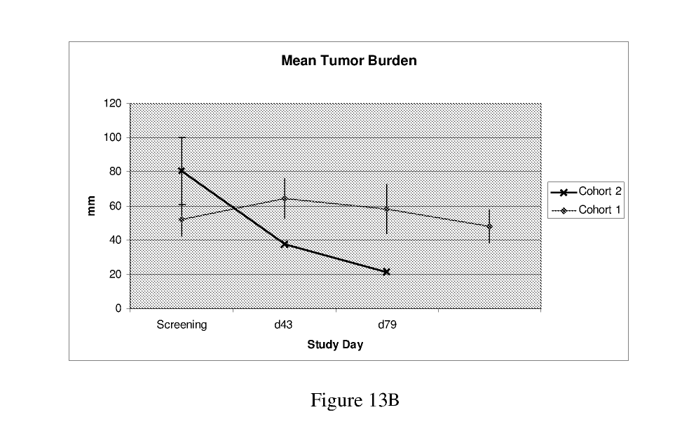

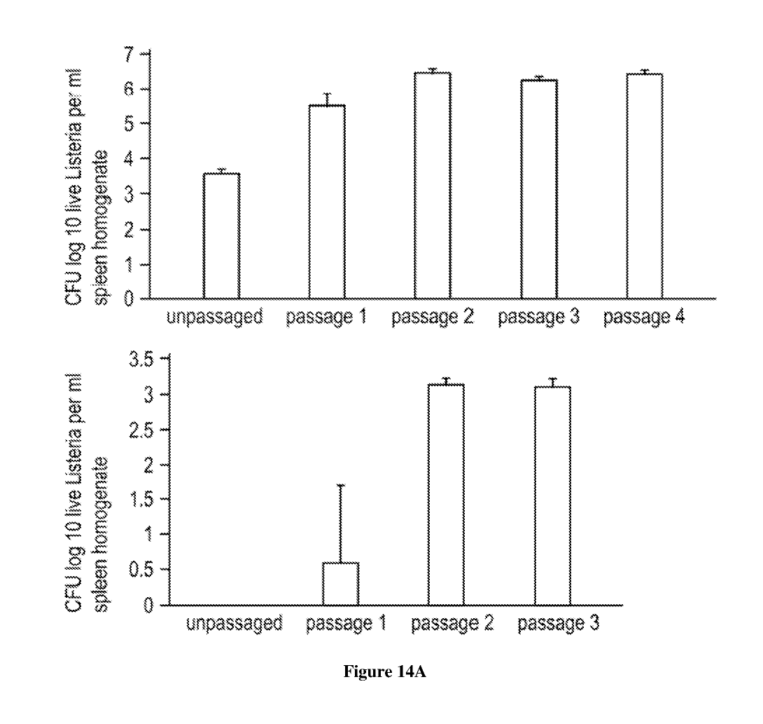

Tumors loads for the 2 cohorts are depicted in FIG. 13B. In summary, even relatively low doses of LM-LLO-E7, administered in a therapeutic regimen containing a priming injection and a single boost, achieved 3 objective responses out of 6 patients for whom data has been collected.

### Discussion

At this late stage of cervical cancer, 1 year survival is typically 10-15% of patients and no tumor therapy has ever been effective. No treatment has shown to be effective in reversing stage IVB cervical cancer. Despite the difficulty of treating cervical cancer at this stage, an anti-tumor effect was observed in 2/6 patients. In addition, other indications of efficacy were observed in patients that finished the trial, as described hereinabove.

Thus, LM-LLO-E7 is safe in human subjects and improves clinical indicators of cervical cancer patients, even when administered at relatively low doses. Additional positive results are likely to be observed when the dose and number of booster vaccinations is increased; and/or when antibiotics are administered in smaller doses or at a later time point after infusion. Pre-clinical studies have shown that a dose increase of a single order of magnitude can cause dramatic changes in response rate (e.g. a change from 0% response rate to 50-100% complete remission rate. Additional booster doses are also very likely to further enhance the immune responses obtained. Moreover, the positive effects of the therapeutic immune response observed are likely to continue with the passage of additional time, as the immune system continues to attack the cancer.

### EXAMPLE 11

### Safety And Efficacy Of LM-LLO-E7 for the Treatment of Cervical Intraepithelial Neoplasia Stages II and III

### Materials and Experimental Methods

### Inclusion Criteria

Age 18 or older and capable of providing informed consent according to federal, state and institutional guidelines.

Patients must have either Stage II or Stage III Cervical Intraepithelial Neoplasia for which surgical intervention is indicated, and for whom the disease is sufficiently indolent to allow for a 6-month treatment and observation period to occur prior to surgery.

HPV-16 E7 positive.

Cytological evidence consistent with a diagnosis of CIN II/III.

All patients eligible for this study must be discussed with the principal investigators and be approved by the principal investigators before study entry.

Patients must respond positively to at least 1 of the test agents used in the anergy panel described for the previous Example. A positive reaction defined by the formation of a local tissue response of at least 5 mm in sum of the orthogonal measures in reaction to the administration of a delayed hypersensitivity stimulus is required.

### Exclusion Criteria

Patients who have had chemotherapy, radiotherapy, or steroids within 4 weeks prior to the initial study dose or those who have not recovered from adverse events due to agents administered more than 4 weeks earlier.

Patients who have received any other investigational agents for 28 days prior to dosing.

A history of Listeriosis.

A history of prior cancer or concomitant cancer.

Patients who are immunocompromised as demonstrated by a negative result from an anergy panel screening.

Uncontrolled intercurrent illness including, but not limited to ongoing or active infection, symptomatic congestive heart failure, unstable angina pectoris, cardiac arrhythmia, or psychiatric illness/social situations that would limit compliance with study requirements.

Hepatitis, cirrhosis, or any other impaired hepatic function as determined by serum enzymes.

Pregnant women and women actively trying to become pregnant.

Known HIV-positive patients.

Penicillin allergy.

Primary Safety Endpoints:

Incidence and severity of observations of the administration site including swelling, irritation, immune reaction or other abnormalities.

Incidence and severity of adverse events assessed throughout the duration of the study.

Changes in clinical hematology and serum chemistry test results at each time point from dosing through week 16.

Rate of clearance of LM-LLO-E7 from the blood, as determined by quantitative blood cultures during the inpatient portion of the study following the initial administration.

Primary Efficacy Endpoints:

Regression of CIN to normal upon colposcopic examination

Regression of CIN toward normal sufficient to cancel or delay surgery

Improved cytology subsequent to surgery

Primary Immunogenicity Endpoints:

HLA typing of patients for Class I and II,

Quantification of a serum cytokine profile subsequent to dosing that corresponds with observed side effects,

Quantification of macrophage activation parameters that assess macrophage activation subsequent to dosing,

Identification of tumor-associated antigen (TAA)-specific activated T cells and quantification of T cell responses subsequent to dosing,

Quantification of T cell subsets migrating to TAA DTH.

Immunogenicity Criteria:

### Serum Cytokines

IFN-γ, TNF-α, IL-2 & IL-12 are assessed in serum of patients, collected at the following times:
Screening, Day 1.
Day 1, pre-dose, Day 1, 3 h post-dose, Day 1, 12 h post-dose, Day 2, 24 h post-dose, and Day 5.
Day 22 pre-dose, Day 22, 3 h post-dose, Day 22, 12 h post-dose, Day 23, 24 h post-dose, and Day 26
Day 43 pre-dose, Day 43, 3 h post-dose, Day 43, 12 h post-dose, Day 44, 24 h post-dose, and Day 47

### T Cell Responses

The following cytokine release profiles are assessed HPV-16 E7 stimulated T cells of patients: IFN-γ, TNF-α, IL-2 & IL-4
Assays are performed on cells sampled from patients at the following times: Screening, Day 1 pre-dosing, day 22 pre-dosing, day 43 pre-dosing, day 126, and day 180

### Delayed Type Hypersensitivity Testing

DTH testing is conducted on the following study days: Screening, Day 5, Day 26, Day 47, Day 126 and Day 180.

### Macrophage Activation

Samples for the assessment of macrophage activation are collected on the following study days and times:

Day 1 pre-dose, Day 1, 3 h post-dose, Day 1, 12 h post-dose, Day 2, 24 h post-dose, and Day 5.

Day 22 pre-dose, Day 22, 3 h post-dose, Day 22, 12 h post-dose, Day 23, 24 h post-dose, and Day 26.

Day 43 pre-dose, Day 43, 3 h post-dose, Day 43, 12 h post-dose, Day 44, 24 h post-dose, and Day 47.

### Vaccine Administration

LM-LLO-E7 is administered as a 30 min. i.v. infusion with each dose freshly thawed and diluted in 250 ml normal saline.

### Safety Review

Adverse Events are graded based on the National Cancer Institute (NCI) Common Toxicity Criteria. Dose limiting toxicity is defined as any of the following:

### Non-Hematologic Toxicity:

1. Presumptive bacterial meningitis as determined by symptoms.
2. Persistent listeremia at day 5 and 15 after a 10-day course of antibiotics.
3. Clinical sepsis requiring ICU admission.
4. A drop in blood pressure sufficient to warrant therapeutic intervention,
5. Hepatitis as evidenced by grade 3-4 elevation in transaminases for a minimum of 7 days.
6. Gastrointestinal toxicity of grade 3-4 despite adequate medical intervention.
7. Any Grade 3 injection site reaction.
8. Any Grade 3 or higher adverse event that cannot be attributed to cervical cancer or other concurrent illnesses.

### Hematologic Toxicity:

1. Absolute neutrophil count (ANC) grade 4 for a minimum of 7 days or neutropenic fever defined as Grade 4 neutropenia with temperature of ≥38.5° C.
2. Platelet count grade 4 or bleeding with Grade 3 platelet count.

Dose escalation to the next cohort proceeds in each case, provided that there are no Grade 3 or higher adverse events related to the therapeutic vaccine.

### Results

Women are enrolled that have stage II or stage III Cervical Intraepithelial Neoplasia (CIN II/III) who have disease that is sufficiently indolent to allow for a 6 month period of treatment and evaluation to occur prior to surgery. Patients receive 3 doses of LM-LLO-E7 at 3 week intervals as inpatients and return for follow up visits to assess their response to the vaccine, collect samples for analysis, and assess their disease. Samples for immunologic analysis are collected throughout the trial and assayed upon the completion of the study.

Safety is assessed through standard physical, hematologic and serum chemistry measures, and by blood cultures to assess serum Listeria. Immunologic activity is assessed in the areas of serum cytokine release, activated T cell responses to tumor antigen, macrophage activation, and delayed hypersensitivity responses (DTH) to tumor antigen.

Clinically, patients are grouped by primary endpoints. Namely, whether patients exhibit sufficient remission of their disease to make surgery unnecessary. Patients that do require surgery, are grouped regarding whether they exhibit lesser disease than the control group. LM-LLO-E7 reduces the fraction of women that subsequently require surgery and/or the degree of disease among those that require surgery.

### EXAMPLE 12

### Passaging of Listeria Vaccine Vectors through Mice Elicits Increased Immune Responses to Heterologous and Endogenous Antigens

### Materials and Experimental Methods

### Bacterial Strains

L. monocytogenes strain 10403S, serotype 1 (ATCC, Manassas, Va.) was the wild type organism used in these studies and the parental strain of the constructs described below. Strain 10403S has an LD₅₀ of approximately 5×10⁴ CFU when injected intraperitoneally into BALB/c mice. “Lm-Gag” is a recombinant LM strain containing a copy of the HIV-1 strain HXB (subtype B laboratory strain with a syncytia-forming phenotype) gag gene stably integrated into the listerial chromosome using a modified shuttle vector pKSV7. Gag protein was expressed and secreted by the strain, as determined by Western blot. All strains were grown in brain-heart infusion (BHI) broth or agar plates (Difco Labs, Detroit, Mich.).

### Bacterial Culture

Bacteria from a single clone expressing the passenger antigen and/or fusion protein were selected and cultured in BHI broth overnight. Aliquots of this culture were frozen at −70° C. with no additives. From this stock, cultures were grown to 0.1-0.2 O.D. at 600 nm, and aliquots were again frozen at −70° C. with no additives. To prepare cloned bacterial pools, the above procedure was used, but after each passage a number of bacterial clones were selected and checked for expression of the target antigen, as described herein. Clones in which expression of the foreign antigen was confirmed were used for the next passage.

### Passage of Bacteria in Mice

6-8 week old female BALB/c (H-2d) mice were purchased from Jackson Laboratories (Bar Harbor, Me.) and were maintained in a pathogen-free microisolator environment. The titer of viable bacteria in an aliquot of stock culture, stored frozen at −70° C., was determined by plating on BHI agar plates on thawing and prior to use. In all, 5×10⁵ bacteria were injected intravenously into BALB/c mice. After 3 days, spleens were harvested, homogenized, and serial dilutions of the spleen homogenate were incubated in BHI broth overnight and plated on BHI agar plates. For further passage, aliquots were again grown to 0.1-0.2 O.D., frozen at −70° C., and bacterial titer was again determined by serial dilution. After the initial passage (passage 0), this sequence was repeated for a total of 4 times.

### Intracellular Cytokine Stain for IFN-Gamma

Lymphocytes were cultured for 5 hours in complete RPMI-10 medium supplemented with 50 U/ml human recombinant IL-2 and 1 microliter/ml Brefeldin A (Golgistop™; PharMingen, San Diego, Calif.) in the presence or absence of either the cytotoxic T-cell (CTL) epitope for HIV-GAG (AMQMLKETI; SEQ ID No: 34), Listeria LLO (GYKDGNEYI; SEQ ID No: 35) or the HPV virus gene E7 (RAHYNIVTF (SEQ ID No: 33), at a concentration of 1 micromole. Cells were first surface-stained, then washed and subjected to intracellular cytokine stain using the Cytofix/Cytoperm kit in accordance with the manufacturer's recommendations (PharMingen, San Diego, Calif.). For intracellular IFN-gamma stain, FITC-conjugated rat anti-mouse IFN-gamma monoclonal antibody (clone XMG 1.2) and its isotype control Ab (rat IgG1; both from PharMingen) was used. In all, 10⁶ cells were stained in PBS containing 1% Bovine Serum Albumin and 0.02% sodium azide (FACS Buffer) for 30 minutes at 4° C. followed by 3 washes in FACS buffer. Sample data were acquired on either a FACScan™ flowcytometer or FACSCalibur™ instrument (Becton Dickinson, San Jose, Calif.). Three-color flow cytometry for CD8 (PERCP conjugated, rat anti-mouse, clone 53-6.7 Pharmingen, San Diego, Calif.), CD62L (APC conjugated, rat anti-mouse, clone MEL-14), and intracellular IFN-gamma was performed using a FACSCalibur™ flow cytometer, and data were further analyzed with CELLQuest software (Becton Dickinson, Mountain View, Calif.). Cells were gated on CD8 high and CD62L^{low} before they were analyzed for CD8⁺ and intracellular IFN-gamma staining.

### Results

### Passaging in Mice Increases the Virulence of Recombinant Listeria Monocytogenes

Three different constructs were used to determine the impact of passaging on recombinant Listeria vaccine vectors. Two of these constructs carry a genomic insertion of the passenger antigen: the first comprises the HIV gag gene (Lm-Gag), and the second comprises the HPV E7 gene (Lm-E7). The third (Lm-LLO-E7) comprises a plasmid with the fusion gene for the passenger antigen (HPV E7) fused with a truncated version of LLO and a gene encoding prfA, the positive regulatory factor that controls Listeria virulence factors. This plasmid was used to complement a prfA negative mutant so that in a live host, selection pressures would favor conservation of the plasmid, because without it the bacterium is avirulent. All 3 constructs had been propagated extensively in vitro for many bacterial generations.

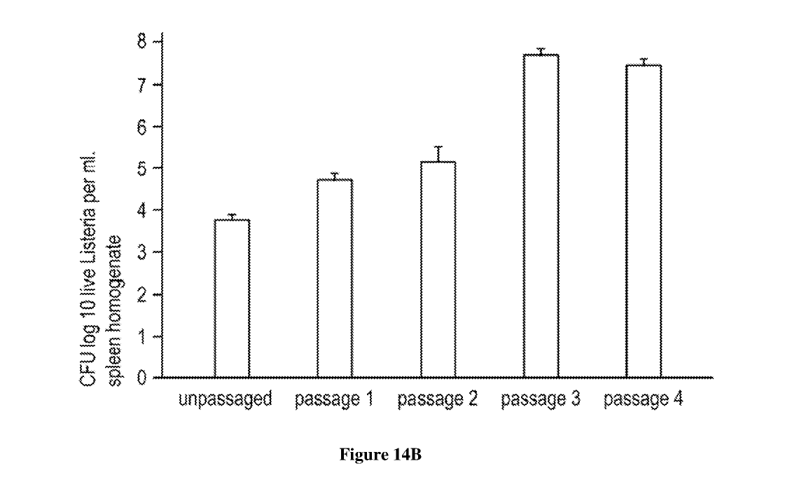

Passaging the bacteria resulted in an increase in bacterial virulence, as measured by numbers of surviving bacteria in the spleen, with each of the first 2 passages. For Lm-Gag and Lm-LLO-E7, virulence increased with each passage up to passage 2 (FIG. 14A). The plasmid-containing construct, Lm-LLO-E7, demonstrated the most dramatic increase in virulence. Prior to passage, the initial immunizing dose of Lm-LLO-E7 had to be increased to 10⁷ bacteria and the spleen had to be harvested on day 2 in order to recover bacteria (whereas an initial dose of 10⁵ bacteria for Lm-Gag was harvested on day 3). After the initial passage, the standard dosage of Lm-LLO-E7 was sufficient to allow harvesting on day 3. For Lm-E7, virulence increased by 1.5 orders of magnitude over unpassaged bacteria (FIG. 14B).

Thus, passage through mice increases the virulence of Listeria vaccine strains.

### Passaging Increases the Ability of L. Monocytogenes to Induce CD8⁺ T Cells

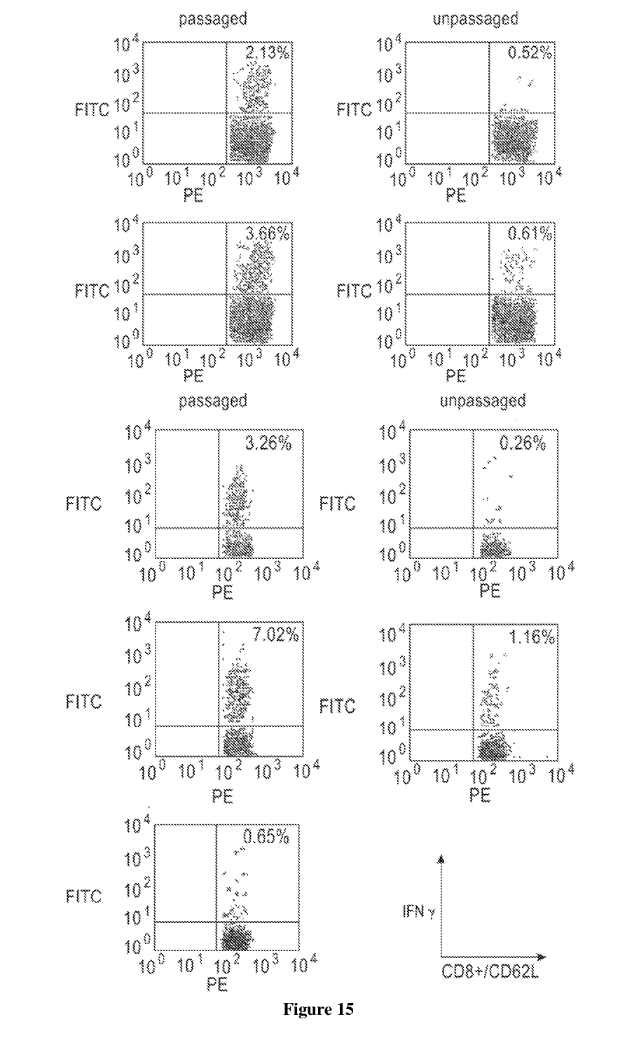

Next, the effect of passaging on induction of antigen-specific CD8⁺ T cells was determined by intracellular cytokine staining with immunodominant peptides specific for MHC-class I using HIV-Gag peptide AMQMLKETI (SEQ ID No: 34) and LLO 91-99 (GYKDGNEYI; SEQ ID No: 35). Injection of 10³ CFU passaged bacteria (Lm-Gag) into mice elicited significant numbers of HIV-Gag-specific CD8⁺ T cells, while the same dose of non-passaged Lm-Gag induced no detectable Gag-specific CD8⁺ T cells. Even increasing the dose of unpassaged bacteria 100-fold did not compensate for their relative avirulence; in fact, no detectable Gag-specific CD8⁺ T cells were elicited even at the higher dose. The same dose increase with passaged bacteria increased Gag-specific T cell induction by 50% (FIG. 15). The same pattern of induction of antigen-specific CD8⁺ T cells was observed with LLO-specific CD8⁺ T cells, showing that these results were not caused by the properties of the passenger antigen, since they were observed with LLO, an endogenous Listeria antigen.

Thus, passage through mice increases the immunogenicity of Listeria vaccine strains.

### EXAMPLE 13

### A PrfA-Containing Plasmid is Stable in an LM Strain with a PrfA Deletion in the Absence of Antibiotics

### Materials and Experimental Methods

### Bacteria

L. monocytogenes strain XFL7 contains a 300 base pair deletion in the prfA gene XFL7 carries pGG55 which partially restores virulence and confers CAP resistance, and is described in United States Patent Application Publication No. 200500118184.

Development of Protocol for Plasmid Extraction from Listeria

1 mL of Listeria monocytogenes Lm-LLO-E7 research working cell bank vial was inoculated into 27 mL BH1 medium containing 34 μg/mL CAP and grown for 24 hours at 37° C. and 200 rpm.

Seven 2.5 mL samples of the culture were pelleted (15000 rpm for 5 minutes), and pellets were incubated at 37° C. with 50μ l lysozyme solution for varying amounts of time, from 0-60 minutes.

Lysozyme Solution:
29μ l 1 M dibasic Potassium Phosphate
21μ l 1 M monobasic Potassium Phosphate
500μ l 40% Sucrose (filter sterilized through 0.45/μm filter)
450μ l water
60 μl lysozyme (50 mg/mL)

After incubation with the lysozyme, the suspensions were centrifuged as before and the supernatants discarded. Each pellet was then subjected to plasmid extraction by a modified version of the QIAprep Spin Miniprep Kit® (Qiagen, Germantown, Md.) protocol. The changes to the protocol were as follows:
1. The volumes of buffers PI, P2 and N3 were all increased threefold to allow complete lysis of the increased biomass.
2. 2 mg/mL of lysozyme was added to the resuspended cells before the addition of P2. The lysis solution was then incubated at 37° C. for 15 minutes before neutralization.
3. The plasmid DNA was resuspended in 30μ L rather than 50μ L to increase the concentration.

In other experiments, the cells were incubated for 15 min in P1 buffer+Lysozyme, then incubated with P2 (lysis buffer) and P3 (neutraliztion buffer) at room temperature.

Equal volumes of the isolated plasmid DNA from each subculture were run on an 0.8% agarose gel stained with ethidium bromide and visualized for any signs of structural or segregation instability.

The results showed that plasmid extraction from L. monocytogenes Lm-LLO-E7 increases in efficiency with increasing incubation time with lysozyme, up to an optimum level at approximately 50 minutes incubation.

These results provide an effective method for plasmid extraction from Listeria vaccine strains.

### Replica Plating

Dilutions of the original culture were plated onto plates containing LB or TB agar in the absence or presence of 34μ g/mL CAP. The differences between the counts on selective and non-selective agar were used to determine whether there was any gross segregational instability of the plasmid.

### Results

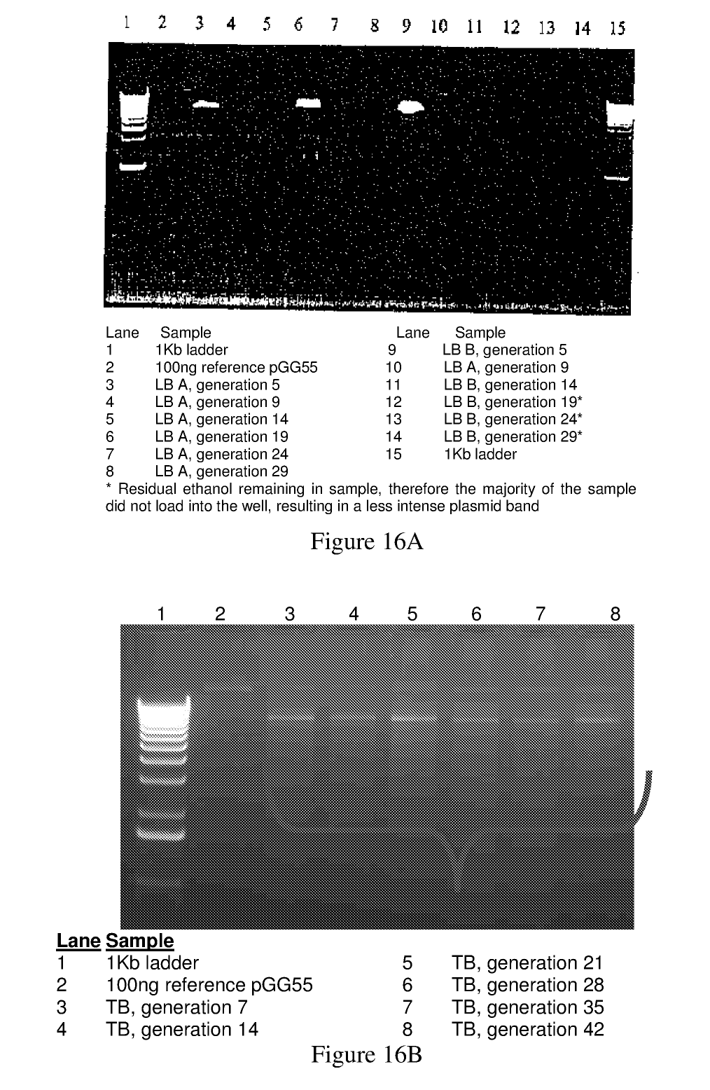

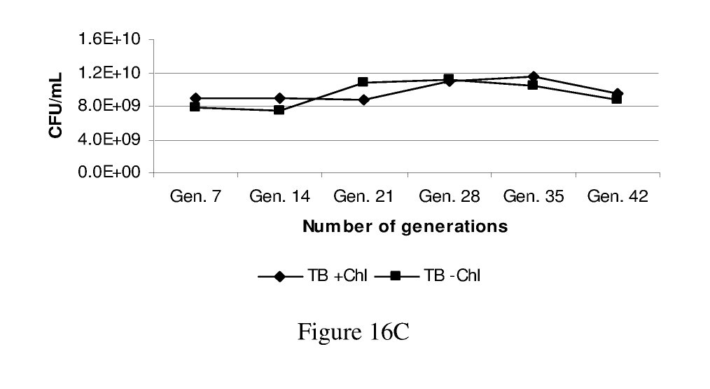

The genetic stability (i.e. the extent to which the plasmid is retained by or remains stably associated with the bacteria in the absence of selection pressure; e.g. antibiotic selection pressure) of the pGG55 plasmid in L. monocytogenes strain XFL7 in the absence of antibiotic was assessed by serial sub-culture in both Luria-Bertani media (LB: 5 g/L NaCl, 10 g/ml soy peptone, 5 g/L yeast extract) and Terrific Broth media (TB: 10 g/L glucose, 11.8 g/L soy peptone, 23.6 g/L yeast extract, 2.2 g/L KH₂PO₄, 9.4 g/L K₂HPO₄), in duplicate cultures. 50 mL of fresh media in a 250 mL baffled shake flask was inoculated with a fixed number of cells (1 ODmL), which was then subcultured at 24 hour intervals. Cultures were incubated in an orbital shaker at 37° C. and 200 rpm. At each subculture the OD₆₀₀ was measured and used to calculate the cell doubling time (or generation) elapsed, until 30 generations were reached in LB and 42 in TB. A known number of cells (15 ODmL) at each subculture stage (approximately every 4 generations) were pelleted by centrifugation, and the plasmid DNA was extracted using the Qiagen QIAprep Spin Miniprep® protocol described above. After purification, plasmid DNA was subjected to agarose gel electrophoresis, followed by ethidium bromide staining. While the amount of plasmid in the preps varied slightly between samples, the overall trend was a constant amount of plasmid with respect to the generational number of the bacteria (FIGS. 16A-16C). Thus, pGG55 exhibited stability in strain XFL7, even in the absence of antibiotic.

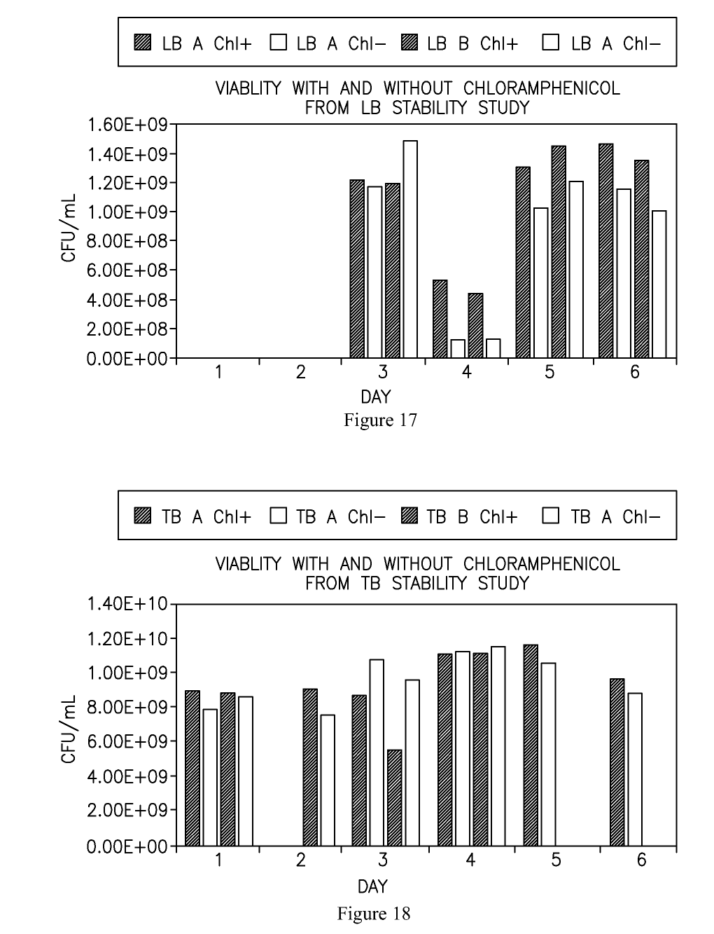

Plasmid stability was also monitored during the stability study by replica plating on agar plates at each stage of the subculture. Consistent with the results from the agarose gel electrophoresis, there was no overall change in the number of plasmid-containing cells throughout the study in either LB or TB liquid culture (FIGS. 17 and 18, respectively). The genetic stability of the pGG55 plasmid in Lm-LLO-E7 showed no signs of structural or segregational instability after 35 or 42 cell generations.

These findings demonstrate that prfA-encoding plasmids exhibit stability in the absence of antibiotic in Listeria strains containing mutations in prfA.

### EXAMPLE 14

### ADXS11-001—Manufacturing Process

Process Overview

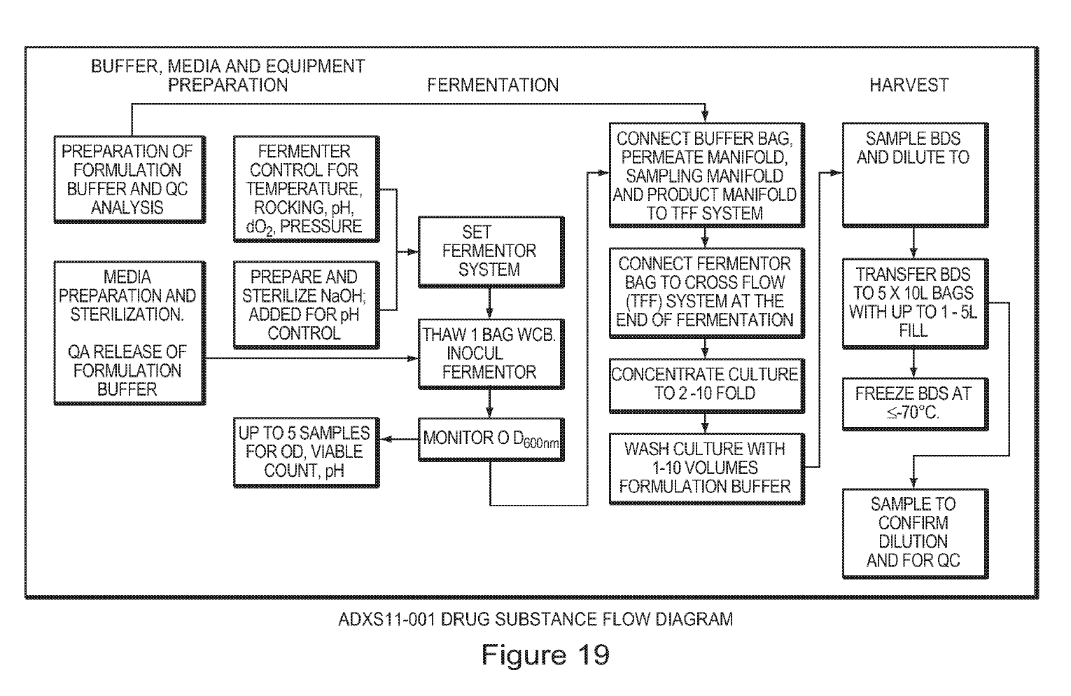

The ADXS11-001 drug substance manufacturing process (FIG. 19) consists of four major steps:
1. Media Preparation
2. Buffer Preparation
3. Fermentation
4. Harvest

Media Preparation and Fermentation: The first part of the process begins with the preparation of the Fermentation Media, Chloramphenicol Stock Solution and 1M NaOH (pH control) for the Fermentation process. After the setup of the fermentation system and the aseptic addition of the Fermentation Media, one 50-120 mL Working Cell Bank (WCB) bag is removed from ≤−70° C. storage and thawed at room temperature for x period of time. The dissolved oxygen (dO2), pH and temperature control loops are initiated to control the fermentation process. The WCB is then aseptically transferred into the fermenter. The dO2 is monitored during the exponential growth and the fermenter is aseptically sampled to measure Optical Density at 600 nm (OD600 nm), pH and Viable Cell Count off-line. Once the target OD600 nm is achieved, all control loops are stopped and the fermentation process is completed.

Buffer Preparation and Harvest: The second part of the process begins with the preparation of the Formulation Buffer which is used for diafiltration and dilution of the drug substance. Once the Tangential Flow Filtration (TFF) system is step up for concentration, the fermenter is aseptically connected to the inlet of the TFF system. The drug substance is then concentrated 2-10 fold by collecting the calculated amount of permeate. The drug substance is then diafiltered with the Formulation Buffer for a pre-determined minimum of diafiltration volumes. The intermediate drug substance is then transferred to the intermediate bag where it will be aseptically sampled for Optical Density (OD) analysis. The intermediate drug substance is then diluted with the Formulation Buffer to a desired target OD and samples for OD analysis. The bulk drug substance is then aseptically transferred into 5×10 L product bags at 1 kg aliquots with a representative sample taken as well and frozen at ≤−70° C.

TABLE 3
ADXS11-001 Drug Substance Process Conditions

| Stage | Process Step | Conditions |
|---|---|---|
| Fermen- | Thawing of | One 60 mL Bag containing 5-20 mL |
| tation | Working | of inoculum. |
| | Cell Bank. | |
| | Inoculate | 1. Fermenter Set points: |
| | Bioreactor | a. Rocking Rate: 10-20 |
| | | Rocks/Minute |
| | | b. Rocking Angle: 5-15° |
| | | 2. Dissolved Oxygen (DO) Set |
| | | Point: 25-100% |
| | | 3. pH Set Point: 6.5-8.5 |
| | | 4. Temperature Set Point: |
| | | 30-45° C. |
| | Fermentation | 1. At hourly or less intervals, |
| | | record the following in-line |
| | | measurements: |
| | | a. DO |
| | | b. pH |
| | | c. Temperature |
| | | 2. At hourly or less intervals, |
| | | sample the fermentation to measure |
| | | the following off-line: |
| | | a. Optical Density at 600 nm (OD_{600 nm}) |
| | | b. pH |
| | | c. Viable Cell Count (VCC) |
| | | 3. Stop Fermentation once an |
| | | OD_{600 nm} value of 5-10 has been |
| | | reached. |
| Harvest | Concentration | 2-10 Fold concentration |
| | | Recirculation Pump Speed set |
| | | point 1-5 L/min |
| | | Feed Pump Speed set point: |
| | | 1-10 L/hr |
| | | Permeate Pump Speed set point: |
| | | 1-10 L/hr |
| | Diafiltration | Diafiltration against 1-10 volumes |
| | | of Formulation Buffer. |
| | | Recirculation Pump Speed set |
| | | point 1-5 L/min |
| | | Feed Pump Speed set point: 1-10 |
| | | L/hr |
| | | Permeate Pump Speed set point: |
| | | 1-10 L/hr |
| | Dilution | Sample Intermediate Drug |
| | | Substance and perform OD600 nm |
| | | analysis to calculate required amount |
| | | of Formulation Buffer to achieve a |
| | | final OD of 8. |
| | Transfer | Transfer 1000 g ± 100 g of Bulk |
| | | Drug Substance into 10 L Product |
| | | Bags. |
| | ADXS11-001 | Stored in 10 L Bags at −70° C. |
| | Drug Substance | QC testing and release. |

TABLE 4
Media preparation

| | | Component | | | | |
|---|---|---|---|---|---|---|
| | | Weights | Release | | Storage | Expiry |
| Formulation | Components | (g/L) | Criteria | Specification | Conditions | Date |
| Chloramphenicol | Chloramphenicol | 0.25-0.50 g | NA | NA | 2-8° C. | 24 hour |
| Stock Solution | 100% Ethanol | 2-10 mL | | | | |
| Fermentation | Potassium DiHydrogen | 5-15 g | NA | NA | Room | 24 Hours |
| Media | Phosphate (KH2PO4) | | | | Temperature | unfiltered. |
| | DiPotassium Hydrogen | 37.00-57.00 g | | | | week once |
| | Phosphate (K2HPO4) | | | | | filtered. |
| | Hy-Soy | 50.00 g-70.00 | | | | |
| | Bacto Yeast Extract | 100.00-120.00 | | | | |
| | D (+) Glucose | 30.00-55.00 g | | | | |
| | Chloramphenicol Stock | 3-12 mL | | | | |
| | Solution | | | | | |
| | WFI | QS to 5-25 kg | | | | |
| pH Control | 1M NaOH | 250-500 mL | NA | NA | Room | 24 Hours |
| Solution | | | | | Temperature | unfiltered. |
| | | | | | | week once |
| | | | | | | filtered. |

TABLE 5
Buffer preparation

| | | Component | Release | | Storage | Expiry |
|---|---|---|---|---|---|---|
| Formulation | Components | Weights (g/L) | Criteria | Specification | Condition | Date |
| Formulation Buffer | Potassium | 2-6 g | Appearance | Clear, | Room | 24 Hours |
| | DiHydrogen | | of Solution | colorless and | Temperature | unfiltered. |
| | Phosphate | | | solution free | | weeks once |
| | (KH2PO4), | | | from | | filtered. |
| | | | | particulate | | |
| | | | | matter | | |
| | DiSodium Hydrogen | 15.00-25.00 g | pH of | 6.8-7.8 | | |
| | Orthophosphate | | Solution | | | |
| | (Na2HPO4) | | | | | |
| | Sodium Chloride | 150.0-175.0 g | Isotonicity of | 300-380 mOsmol/kg | | |
| | (NaCl) | | Solution | | | |
| | Potassium | 2.0-6.0 g | Endotoxin | <0.25 EU/mL | | |
| | Chloride (KCl) | | Content | | | |
| | Sucrose | 250-500 g | Sterility | Pass | | |
| | WFI | QS to 5.00-25.00 kg | | | | |

I. Process Description

A. Preparation of the Chloramphenicol Stock Solution for the Fermentation Media

The Chloramphenicol Stock Solution used in the Fermentation Media is prepared using the following components in Table 6.

TABLE 6

| Component | | |
|---|---|---|
| Chloramphenicol | 0.25-0.50 | g |
| Ethanol | 2-10 | mL |

The Chloramphenicol Stock Solution is prepared as a 1×10 mL aliquot and in a Grade C environment. Chloramphenicol is weighed to within 1% of the target weight into a sterile 50 mL centrifuge tube. Then Ethanol is added to the centrifuge tube containing the Chloramphenicol using a pipette. The Chloramphenicol is allowed to dissolve in the Ethanol. The solution is stored at 2-8° C. for a maximum of 24 hours.

A. Preparation of the Fermentation Media

The Fermentation Media used in the Production Fermenter is prepared according to the recipe shown in Table 6.

The Fermentation media is prepared as a 1×5 L aliquot and in a Grade C environment. The solids are weighed out to within 1% of the target weights. The solution is then mixed in some Water For Injection (WFI) (for dissolving solutes) until the solids are fully dissolved. A pipette is then used to accurately dispense 5 mL of Chloramphenicol Stock Solution and then the solution is QS (Quantum Satis) with WFI to 5.00-25.00 kg. The unfiltered Fermentation Media is then filtered using two 0.2 μm filter into a 5 L Media Addition bag. The inlet of the bag is sealed using a sterile welder and the two filters are detached. The filters are integrity tested according to the current manufacturing SOP. The filtered Fermentation Media is stored at room temperature and assigned a one week expiry.

A. Preparation of the 1M NaOH for Fermentation pH Control

The 1M NaOH for pH control is used in the Production Fermenter is prepared according to the recipe shown in Table 8.

TABLE 8

| Component | Amount of Buffer Required (mL) |
|---|---|
| 1M NaOH | 600 |

The 1M NaOH for pH control is prepared as a 1×600 mL aliquot and in a Grade C environment. The unfiltered 1M NaOH for pH control is then filtered using two 0.2 μm filter into the pH Control Bag. The inlet of the bag is sealed using a sterile welder and the two filters are detached. The filters are integrity tested according to the current manufacturing SOP. The filtered 1M NaOH is stored at room temperature and assigned a one week expiry.

A. Preparation of the Formulation Buffer

The Formulation Buffer used in the Diafiltration and Final Dilution steps is prepared according to the recipe shown in Table 6.

The Formulation Buffer is prepared as 2×500 mL in 1 L bags and 1×20 L in 20 L bag aliquots and in a Grade C environment. First, collect approximately 15 L of WFI into a suitably sized empty sterile vessel. The solids are weighed out to within 1% of the target weights and then added to the vessel. Mix the contents of the vessel until the solids are fully dissolved. The solution is then QS (Quantum Satis) with WFI to 21 Kg. The pH of the buffer is measured and is ensured it is within the range of 7-8. The unfiltered Formulation Buffer is then filtered using two 0.2 μm filter into 2×1 L bags and a 20 L bag. The inlet of the bags are sealed using a sterile welder and the two filters are detached. The filters are integrity tested according to the current manufacturing SOP. The 2×1 L bags containing 500 mL of Formulation Buffer are submitted for QC analysis for sterility testing. The 20 L aliquot of Formulation Buffer is stored at room temperature and assigned a six week expiry date.

A. Fermenter Preparation

The Fermenter is first prepared by transferring 5 L of the filtered Fermentation Media to the 50 L Culti-bag. The rocker is then activated and set to operate according to the following table.

TABLE 10

| | Setpoint |
|---|---|
| Rocking Rate | 10-20 Rocks/Minute |
| Rocking Angle | 5-15° |

The pH Control Bag containing 600 mL of filtered 1M NaOH is connected to a port on the 50 L Culti-bag using a sterile tube welder. The rocking rate and angle are set to maintain a dissolved oxygen (DO) concentration of 35% at air saturation and the pH is maintained at 7.00±0.4 by the addition of 1M NaOH. The temperature is also set at 37.0° C. The fermenter controls are set to operate within the ranges and set points according to the following table:

TABLE 11

| Parameter | Oxygen | Temperature | pH |
|---|---|---|---|
| S.P. Start | 25-100% | 30-45° C. | 6.50-7.50 |
| Operating | ≥90% | 30-45° C. | 6.50-7.50 |
| Range | | | |

A. Fermentor Inoculation

60 mL Bags containing the 5-20 mL of the ADXS11-001 Working Cell Bank (WCB) are stored at −70±10° C. To initiate production of ADXS11-001, a 60 mL bag of the WCB is removed from storage, brought into the manufacturing suite, thawed at room temperature, and the contents used to inoculate an 50 L Culti-bag containing 5 L of the sterile Fermentation Media.

G. Growth in Production Fermentor

During fermentation and at regular intervals, the in-line DO, pH and temperature are recorded. In addition, samples are taken at regular intervals to measure the Optical Density at 600 nm (OD₆₀₀), off-line pH and Viable Cell Count (VCC). The fermentation is monitored and when the culture reaches a target OD₆₀₀ of about 5-10 units, the fermentation is stopped.

H. Harvest of Culture

At the completion of the fermentation process, the total fermentation volume of about 5 L, containing approximately X kg biomass), the 50 L Culti-bag containing the culture is connected to the feed line of the Tangential Flow Filtration (TFF) System using a sterile tube welder. The cartridges are subject to pre and post integrity testing. A 2-10 fold concentration is performed on the harvest.

The concentrated harvest slurry is then diafiltered against the Formulation Buffer to remove low molecular weight contaminants such as media salts from the solution. The slurry is diafiltered against 1-10 volumes of Formulation Buffer.

I. Formulation

Following the diafiltration, the intermediate drug substance is transferred into the Flush Bag and diluted with additional Formulation Buffer. An OD_{600 mm} analysis is performed to calculate the amount of Formulation Buffer that is needed to achieve a final desired OD.

J. Drug Substance Storage

The Bulk Drug Substance (BDS) in the Flush Bag is then transferred to five 10 L Product Bags. A pump is used to aseptically transfer 1000 g±100 g of the Bulk Drug Substance into Product Bags #1 through #4. The remaining Bulk Drug Substance is transferred into the final Product Bag #5. The Product Bags are then transferred into a −70° C. freezer.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A process for the manufacturing of a formulation comprising a drug substance, said drug substance comprising a recombinant Listeria, said recombinant Listeria strain comprising a nucleic acid comprising an open reading frame encoding a recombinant polypeptide, said recombinant polypeptide comprising a human papilloma virus (HPV) antigen fused to a Listeriolysin O (LLO) polypeptide, the method comprising the steps of:
a) Preparing fermentation media and adding said fermentation media into a fermenter system.
i. wherein said preparing further comprises adding an antibiotic agent in solution.
b) Adding a working cell bank (WCB) recombinant Listeria into said fermenter system.
i. initiating the fermentation process until a target optical density (OD) of said drug substance is reached.
c) Aseptically connecting said fermenter system to a filtration system and concentrating said drug substance to a desired weight by collecting a calculated amount of permeate.
d) Obtaining a retentate solution comprising said drug substance from step c) and exchanging the fermentation media with an appropriate formulation for human use
i. measuring the OD of said formulation buffer solution comprising said drug substance.
e) Diluting said drug substance using said formulation buffer to a target OD, thereby forming a bulk drug substance (BDS).
f) Transferring said BDS into intermediate product bags.
i. aseptically filling vials with BDS from said product bags for storage or for drug preparation and administration to a subject.

2. The process of claim 1, wherein said fermentation system is a disposable system.

3. The process of claims 1-2, wherein the pH of said fermentation process is controlled using an alkylating agent.

4. The process of claim 3, wherein said alkylating agent is NaOH.

5. The process of claims 1-4, wherein said WCB is removed from ≤−70° C. storage and thawed at room temperature prior to adding into said fermenter system.

6. The process of claims 1-5, wherein said fermentation process is controlled by monitoring the dissolved oxygen (dO2) levels, the pH, Optical Density at 600 nm (OD_{600 nm}) and temperature within the fermentation system.

7. The process of claims 1-6, wherein said target OD is 5-10 units.

8. The process of claims 1-6, wherein the dO2 is monitored during the exponential growth.

9. The process of claims 1-8, wherein the fermenter is aseptically sampled to measure Optical Density at 600 nm (OD_{600 nm}), pH and Viable Cell Count off-line following transfer of the WCB into said fermenter.

10. The process of claims 1-8, wherein said filtration system is a disposable Tangential Flow Filtration (TFF or CFF) system.

11. The process of claim 10, wherein said fermenter system is aseptically connected to the inlet of said TFF system.

12. The process of claims 1-10, wherein said drug substance is concentrated 2-10 fold.

13. The process of claims 1-12, wherein said exchanging comprises diafiltering said drug substance about 1-10 times with said formulation buffer prior to measuring said OD.

14. The process of claim 13, wherein said product bags are 5×10 L in volume and said transferring is carried out in 1-5 kg aliquots.

15. The process of claims 13-14, wherein said measuring of OD is carried out following aseptically obtaining a sample from said product bag.

16. The process of any one of claims 1-15, wherein said vials are 1-10 ml in volume.

17. The process of any one of claims 1-16, wherein said vial for storage is stored frozen at ≤−70° C.

18. The process of claims 1-17, wherein said HPV antigen is an E6 antigen.

19. The process of claims 1-17, wherein said HPV antigen is an E7 antigen.

20. The process of claims 1-18, wherein said LLO is an N-terminal LLO.

21. The process of claims 1-20, wherein said N-terminal LLO is selected from a sequence comprising SEQ ID NO: 2.

22. The process of claim 1-21, wherein said recombinant Listeria comprises a mutation, deletion or inactivation of an endogenous prfA gene.

23. The recombinant Listeria strain of claims 1-22, wherein said nucleic acid molecule is in a plasmid in said recombinant Listeria strain.

24. The recombinant Listeria strain of claim 23, wherein said plasmid is stably maintained in said recombinant Listeria strain in the absence of antibiotic selection.

25. The recombinant Listeria strain of claim 24, wherein said plasmid does not confer antibiotic resistance upon said recombinant Listeria.

26. The process of claims 23-25, wherein said plasmid comprises an open reading frame encoding a mutant prfA gene.

27. The process of claim 26, wherein said mutant prfA gene encodes a mutant PrfA protein that complements said mutation, inactivation or deletion in said endogenous prfA gene in said recombinant Listeria or restores partial PrfA function in said recombinant Listeria.

28. The process of claim 27, wherein said mutant PrfA protein comprises a D133V amino acid mutation.

29. The process of claims 1-28, wherein said recombinant polypeptide is expressed by said recombinant Listeria.

30. The process of claims 1-29, wherein said Listeria has been passaged through an animal host.

31. The process of claims 1-30, wherein said recombinant Listeria is a Listeria monocytogenes.
